(19) United States
(12) Patent Application Publication
(10) Pub. No.: US 2024/0248037 A1
(43) Pub. Date: Jul. 25, 2024

(54) COMPOSITIONS COMPRISING LIGHT-HARVESTING MOLECULES AND METHODS OF USE THEREOF FOR DETECTING RARE EARTH AND ACTINIDE ELEMENTS

(71) Applicant: Lawrence Livermore National Security, LLC, Livermore, CA (US)

(72) Inventors: Gauthier Deblonde, San Ramon, CA (US); Annie B. Kersting, Oakland, CA (US); Mavrik Zavarin, Oakland, CA (US)

(21) Appl. No.: 18/097,177

(22) Filed: Jan. 13, 2023

(51) Int. Cl. G01N 21/64

(52) U.S. Cl. CPC G01N 21/643

(57) ABSTRACT

This disclosure provides compositions comprising a light-harvesting molecule capable of producing luminescence upon interaction with a rare earth element or an actinide element, and a buffer, as well as assay systems, kits, and methods of detecting the presence of rare earth elements and/or actinide elements.

### STATEMENT REGARDING FEDERALLY SPONSORED RESEARCH

This invention was made with Government support from Lawrence Livermore National Laboratory, which is operated by Lawrence Livermore National Security, LLC, for the U.S. Department of Energy, National Nuclear Security Administration under Contract DE-AC52-07NA27344. This work was funded by the Office of Defense Nuclear Nonproliferation Research and Development. The Government has certain rights in the invention.

### BACKGROUND

Rare earth elements (“REEs”) are mined from the Earth's crust or recycled from secondary sources. Because of their unique physical and chemical properties, these elements are crucial in many industrial sectors (e.g., catalysis, defense, electronics, and optics). REEs are key ingredients in a growing number of high-tech products, including high-performance magnets, hard drives, MRI machines, energy-efficient light bulbs, electric engines, electric vehicle batteries, polishing materials, and lasers, to name a few. Likewise, actinide elements are crucial to many strategic sectors, including civilian nuclear energy production, nuclear medicine, and defense applications.

Technology and instrumentation to analyze rare earth element samples, as well as actinide-containing samples, is rather limited. Analytical methods are often limited to inductively coupled plasma spectroscopy (ICP-MS or ICP-OES), which requires expensive instruments, lengthy protocols, and cannot be performed in-field. Samples are often required to be shipped to dedicated analytical labs for analysis. In addition, the demand for REEs continues to surge at a rapid rate. Accordingly, there remains a need for convenient and inexpensive tools to detect and analyze REEs and actinides.

### SUMMARY

Materials and methods are provided herein for detecting and analyzing REEs and actinide elements. In some embodiments, the present technology provides compositions comprising a light-harvesting molecule capable of producing luminescence upon interaction with a rare earth element and/or an actinide element, the light-harvesting molecule comprising one hydroxypyridinone (HOPO) group having the structure of formula (I) or (II):

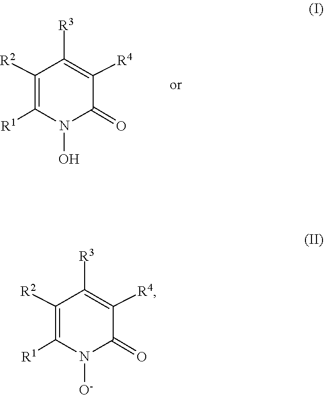

or a salt, solvate, tautomer, or stereoisomer thereof, wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; and a buffer; wherein the composition has a pH between about 6 to about 11.

In some aspects, the light-harvesting molecule lacks an amide group.

In some aspects, the composition is in the form of an aqueous solution.

In some aspects, the composition is in the form of a solution, which further comprises water or an organic solvent. In some aspects, wherein the organic solvent is at least one selected from the group consisting of an alcohol, acetonitrile, dimethylformamide (DMF), and dimethyl sulfoxide (DMSO).

In some aspects, wherein the light-harvesting molecule comprises one hydroxypyridinone (HOPO) group having the structure of formula (I), or a salt, solvate, tautomer, or stereoisomer thereof.

In some aspects, wherein the light-harvesting molecule comprises one hydroxypyridinone (HOPO) group having the structure of formula (II), or a salt, solvate, tautomer, or stereoisomer thereof.

In some aspects, R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; R² is hydrogen; R³ is an alkyl group having from 1 to 6 carbon atoms; and R⁴ is hydrogen.

In some aspects, R³ is a methyl group (—CH₃).

In some aspects, R¹ is an alkyl group having between 1 to 10 carbon atoms.

In some aspects, R¹ is an alkyl group having 8 carbon atoms.

In some aspects, R¹ is —CH₂CH(CH₃)CH₂C(CH₃)₃.

In some aspects, the light-harvesting molecule comprises the structure of piroctone or a salt thereof:

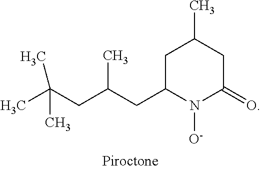 [SMILES: CC1CC(=O)N([O-])C(CC(C)CC(C)(C)C)C1]

In some aspects, piroctone is in the form of piroctone olamine.

In some aspects, R¹ is a cycloalkyl or substituted cycloalkyl group having between 3 and 8 carbon atoms.

In some aspects, R¹ is a cycloalkyl or substituted cycloalkyl group having 6 carbon atoms.

In some aspects, R¹ is cyclohexyl.

In some aspects, the light-harvesting molecule comprises the structure of ciclopirox or a salt thereof:

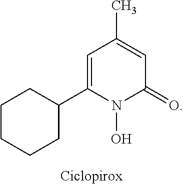

In some aspects, ciclopirox is in the form of ciclopirox olamine.

In some aspects, R¹ is hydrogen; R² is hydrogen; R³ is a hydroxy group (—OH); and R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some aspects, R⁴ is a cycloalkyl or substituted cycloalkyl group having between 5 and 14 carbon atoms.

In some aspects, R⁴ is a cycloalkyl or substituted cycloalkyl group having 10 carbon atoms.

In some aspects, R⁴ is

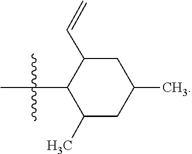 [SMILES: *C1C(C)CC(C)CC1C=C]

In some aspects, the light-harvesting molecule comprises the structure of pyridoxatin, or an enantiomer, or a tautomer thereof:

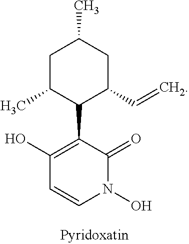

In some aspects, the rare earth element is at least one selected from the group consisting of Sm, Eu, Tb, Dy, Pr, and Ho.

In some aspects, the actinide element is Cm and/or Am.

In some embodiments, the present technology provides compositions comprising a light-harvesting molecule capable of producing luminescence upon interaction with a rare earth element and/or an actinide element, the light-harvesting molecule comprising one hydroxypyridinone (HOPO) group having the structure of formula (I) or (II):

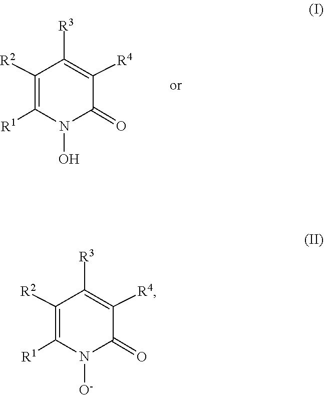

or a salt, solvate, tautomer, or stereoisomer thereof, wherein each of R¹-R⁴ are independently selected from a hydrogen, a hydroxy group, or a hydrocarbon group, wherein the hydrocarbon group is an alkyl group having from 1 to 20 carbon atoms, a substituted alkyl group having from 1 to 20 carbon atoms, a cycloalkyl group having from 3 to 20 carbon atoms, or a substituted cycloalkyl group having from 3 to 20 carbon atoms; and a buffer, wherein the composition has a pH between about 6 to about 11.

In some aspects, R¹ is —CH₂CH(CH₃)CH₂C(CH₃)₃, cyclohexyl, or hydrogen, R² is hydrogen, R³ is a methyl group (—CH₃) or a hydroxy group, and R⁴ is hydrogen or

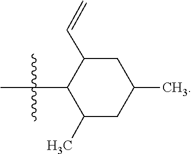 [SMILES: *C1C(C)CC(C)CC1C=C]

In some embodiments, the present technology provides assay systems comprising one or more compositions disclosed herein.

In some embodiments, the present technology provides assay systems comprising a light-harvesting molecule immobilized on an assay test strip, wherein the light-harvesting molecule is capable of producing luminescence upon interaction with a rare earth element and/or an actinide element, the light-harvesting molecule comprises one hydroxypyridinone (HOPO) group having the structure of formula (I) or (II):

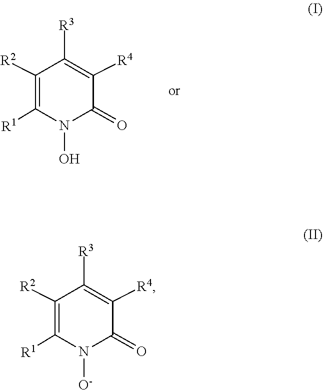

or a salt, solvate, tautomer, or stereoisomer thereof, wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; and the light-harvesting molecule is present in a composition comprising a buffer, wherein the composition has a pH between about 6 to about 11.

In some aspects, the light-harvesting molecule lacks an amide group.

In some aspects, the composition is in the form of an aqueous solution.

In some aspects, the composition is in the form of a solution, which further comprises water or an organic solvent. In some aspects, the organic solvent is at least one selected from the group consisting of an alcohol, acetonitrile, dimethylformamide (DMF), and dimethyl sulfoxide (DMSO).

In some aspects, the light-harvesting molecule comprises one hydroxypyridinone (HOPO) group having the structure of formula (I), or a salt, solvate, tautomer, or stereoisomer thereof.

In some aspects, the light-harvesting molecule comprises one hydroxypyridinone (HOPO) group having the structure of formula (II), or a salt, solvate, tautomer, or stereoisomer thereof.

In some aspects, R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; R² is hydrogen; R³ is an alkyl group having from 1 to 6 carbon atoms; and R⁴ is hydrogen.

In some aspects, R³ is a methyl group (—CH₃).

In some aspects, R¹ is an alkyl group having between 1 to 10 carbon atoms.

In some aspects, R¹ is an alkyl group having 8 carbon atoms.

In some aspects, R¹ is —CH₂CH(CH₃)CH₂C(CH₃)₃.

In some aspects, the light-harvesting molecule comprises the structure of piroctone or a salt thereof:

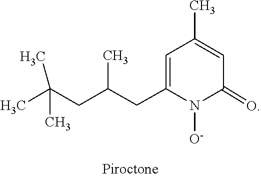 [SMILES: Cc1cc(CC(C)CC(C)(C)C)n([O-])c(=O)c1]

In some aspects, piroctone is in the form of piroctone olamine.

In some aspects, R¹ is a cycloalkyl or substituted cycloalkyl group having between 3 and 8 carbon atoms.

In some aspects, R¹ is a cycloalkyl or substituted cycloalkyl group having 6 carbon atoms.

In some aspects, R¹ is cyclohexyl.

In some aspects, the light-harvesting molecule comprises the structure of ciclopirox or a salt thereof:

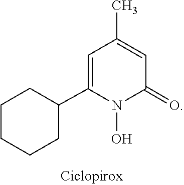

In some aspects, ciclopirox is in the form of ciclopirox olamine.

In some aspects, R¹ is hydrogen; R² is hydrogen; R³ is a hydroxy group (—OH); and R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some aspects, R⁴ is a cycloalkyl or substituted cycloalkyl group having between 5 and 14 carbon atoms.

In some aspects, R⁴ is a cycloalkyl or substituted cycloalkyl group having 10 carbon atoms.

In some aspects, R⁴ is

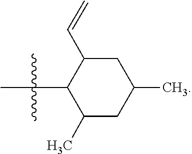 [SMILES: *C1C(C)CC(C)CC1C=C]

In some aspects, the light-harvesting molecule comprises the structure of pyridoxatin, or an enantiomer, or a tautomer thereof:

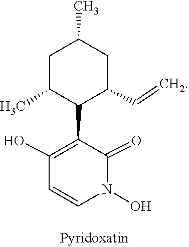

In some aspects, the rare earth element is at least one selected from the group consisting of Sm, Eu, Tb, Dy, Pr, and Ho.

In some aspects, the actinide element is Cm and/or Am.

In some embodiments, the present technology provides a kit comprising a reagent comprising one or more compositions disclosed herein and one or more containers for combining an aliquot of the reagent with a test sample.

In some embodiments, the present technology provides a kit comprising a light-harvesting molecule immobilized on an assay test strip, wherein the light-harvesting molecule is capable of producing luminescence upon interaction with a rare earth element and/or an actinide element, the light-harvesting molecule comprises one hydroxypyridinone (HOPO) group having the structure of formula (I) or (II):

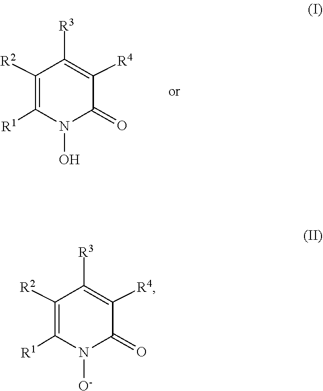

or a salt, solvate, tautomer, or stereoisomer thereof, wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; and optionally a solution comprising a buffer for dissolving a test sample comprising a rare earth element and/or an actinide element.

In some aspects, the solution is present, the kit further comprises one or more containers for combining an aliquot of the solution with the test sample.

In some aspects, the light-harvesting molecule lacks an amide group.

In some aspects, the solution is present, and the solution is an aqueous solution.

In some aspects, the solution is present, and the solution comprises water or an organic solvent. In some aspects, the organic solvent is at least one selected from the group consisting of an alcohol, acetonitrile, dimethylformamide (DMF), and dimethyl sulfoxide (DMSO).

In some aspects, the light-harvesting molecule comprises one hydroxypyridinone (HOPO) group having the structure of formula (I), or a salt, solvate, tautomer, or stereoisomer thereof.

In some aspects, the light-harvesting molecule comprises one hydroxypyridinone (HOPO) group having the structure of formula (II), or a salt, solvate, tautomer, or stereoisomer thereof.

In some aspects, R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; R² is hydrogen; R³ is an alkyl group having from 1 to 6 carbon atoms; and R⁴ is hydrogen.

In some aspects, R³ is a methyl group (—CH₃).

In some aspects, R¹ is an alkyl group having between 1 to 10 carbon atoms.

In some aspects, R¹ is an alkyl group having 8 carbon atoms.

In some aspects, R¹ is —CH₂CH(CH₃)CH₂C(CH₃)₃.

In some aspects, the light-harvesting molecule comprises the structure of piroctone or a salt thereof:

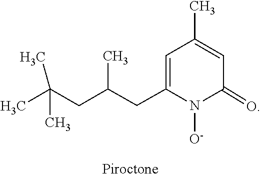 [SMILES: Cc1cc(CC(C)CC(C)(C)C)n([O-])c(=O)c1]

In some aspects, piroctone is in the form of piroctone olamine.

In some aspects, R¹ is a cycloalkyl or substituted cycloalkyl group having between 3 and 8 carbon atoms.

In some aspects, R¹ is a cycloalkyl or substituted cycloalkyl group having 6 carbon atoms.

In some aspects, R¹ is cyclohexyl.

In some aspects, the light-harvesting molecule comprises the structure of ciclopirox or a salt thereof:

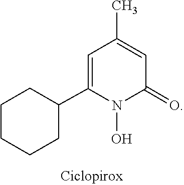

In some aspects, ciclopirox is in the form of ciclopirox olamine.

In some aspects, R¹ is hydrogen; R² is hydrogen; R³ is a hydroxy group (—OH); and R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some aspects, R⁴ is a cycloalkyl or substituted cycloalkyl group having between 5 and 14 carbon atoms.

In some aspects, R⁴ is a cycloalkyl or substituted cycloalkyl group having 10 carbon atoms.

In some aspects, R⁴ is

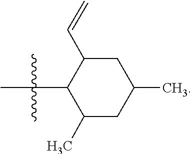 [SMILES: *C1C(C)CC(C)CC1C=C]

In some aspects, the light-harvesting molecule comprises the structure of pyridoxatin, or an enantiomer, or a tautomer thereof:

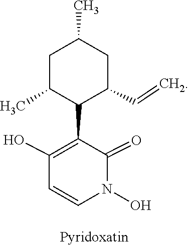

In some aspects, the rare earth element is at least one selected from the group consisting of Sm, Eu, Tb, Dy, Pr, and Ho.

In some aspects, the actinide element is Cm and/or Am.

In some aspects, the kit further comprises a light source that emits ultraviolet (UV) light with a wavelength of less than 400 nm.

In some aspects, the kit further comprises a luminescence detector.

In some embodiments, the present technology provides methods for detecting the presence of a rare earth element and/or an actinide element in a test sample. The method comprises: combining the test sample with the composition of any one of claims 1-26 to form a test mixture; exposing the test mixture to UV light with a wavelength of less than 400 nm; and determining that the rare earth element and/or the actinide element is present in the test sample when a luminescent signal is detected by a luminescence detector.

In some aspects, the luminescent signal is a fluorescence emission.

In some aspects, the rare earth element is at least one selected from the group consisting of Sm, Eu, Tb, Dy, Pr, and Ho.

In some aspects, the actinide element is Cm and/or Am.

In some aspects, the rare earth element is Sm, wherein a fluorescence emission at 630-670 nm is detected upon excitation at a wavelength of less than 400 nm, and in certain aspects, between 290-330 nm.

In some aspects, the rare earth element is Eu, wherein a fluorescence emission at 600-630 nm is detected upon excitation at a wavelength of less than 400 nm, and in certain aspects, between 290-330 nm.

In some aspects, the rare earth element is Tb, wherein a fluorescence emission at 520-560 nm is detected upon excitation at a wavelength of less than 400 nm, and in certain aspects, between 290-330 nm.

In some aspects, the rare earth element is Dy, wherein a fluorescence emission at 550-590 nm is detected upon excitation at a wavelength of less than 400 nm, and in certain aspects, between 290-330 nm.

In some aspects, the test mixture further comprises water or an organic solvent. In some aspects, the organic solvent is at least one selected from the group consisting of an alcohol, acetonitrile, dimethylformamide (DMF), and dimethyl sulfoxide (DMSO).

In some aspects, the test mixture has a pH between about 6 to about 11.

In some aspects, the light-harvesting molecule is present in the test mixture at a concentration of 1-50 μM.

In some embodiments, the present technology provides methods for detecting the presence of a rare earth element and/or an actinide element in a test sample. The method comprises: combining the test sample with a light-harvesting molecule capable of producing luminescence upon interaction with a rare earth element and/or an actinide element to form a solid test mixture; exposing the solid test mixture to UV light with a wavelength of less than 400 nm; and determining that the rare earth element and/or the actinide element is present in the test sample when a luminescent signal is detected by a luminescence detector, wherein the light-harvesting molecule comprises one hydroxypyridinone (HOPO) group having the structure of formula (I) or (II):

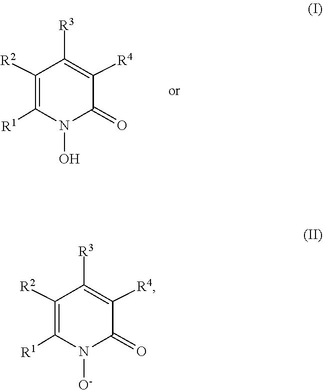

or a salt, solvate, tautomer, or stereoisomer thereof, wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some aspects, the light-harvesting molecule lacks an amide group.

In some aspects, the light-harvesting molecule comprises one hydroxypyridinone (HOPO) group having the structure of formula (I), or a salt, solvate, tautomer, or stereoisomer thereof.

In some aspects, the light-harvesting molecule comprises one hydroxypyridinone (HOPO) group having the structure of formula (II), or a salt, solvate, tautomer, or stereoisomer thereof.

In some aspects, R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; R² is hydrogen; R³ is an alkyl group having from 1 to 6 carbon atoms; and R⁴ is hydrogen.

In some aspects, R³ is a methyl group (—CH₃).

In some aspects, R¹ is an alkyl group having between 1 to 10 carbon atoms.

In some aspects, R¹ is an alkyl group having 8 carbon atoms.

In some aspects, R¹ is —CH₂CH(CH₃)CH₂C(CH₃)₃.

In some aspects, the light-harvesting molecule comprises the structure of piroctone or a salt thereof:

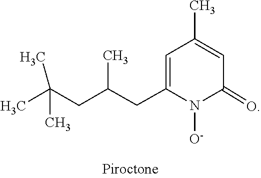 [SMILES: Cc1cc(CC(C)CC(C)(C)C)n([O-])c(=O)c1]

In some aspects, piroctone is in the form of piroctone olamine.

In some aspects, R¹ is a cycloalkyl or substituted cycloalkyl group having between 3 and 8 carbon atoms.

In some aspects, R¹ is a cycloalkyl or substituted cycloalkyl group having 6 carbon atoms.

In some aspects, R¹ is cyclohexyl.

In some aspects, the light-harvesting molecule comprises the structure of ciclopirox or a salt thereof:

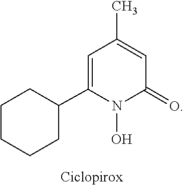

In some aspects, ciclopirox is in the form of ciclopirox olamine.

In some aspects, R¹ is hydrogen; R² is hydrogen; R³ is a hydroxy group (—OH); and R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some aspects, R⁴ is a cycloalkyl or substituted cycloalkyl group having between 5 and 14 carbon atoms.

In some aspects, R⁴ is a cycloalkyl or substituted cycloalkyl group having 10 carbon atoms.

In some aspects, R⁴ is

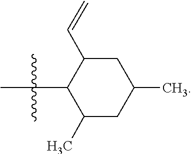 [SMILES: *C1C(C)CC(C)CC1C=C]

In some aspects, the light-harvesting molecule comprises the structure of pyridoxatin, or an enantiomer, or a tautomer thereof:

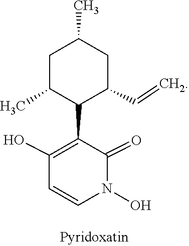

In some aspects, the rare earth element is at least one selected from the group consisting of Sm, Eu, Tb, Dy, Pr, and Ho.

In some aspects, the actinide element is Cm, and/or Am.

In some aspects, the rare earth element is Sm, wherein a fluorescence emission at 630-670 nm is detected upon excitation at a wavelength of less than 400 nm, and in certain aspects, between 290-330 nm.

In some aspects, the rare earth element is Eu, wherein a fluorescence emission at 600-630 nm is detected upon excitation at a wavelength of less than 400 nm, and in certain aspects, between 290-330 nm.

In some aspects, the rare earth element is Tb, wherein a fluorescence emission at 520-560 nm is detected upon excitation at a wavelength of less than 400 nm, and in certain aspects, between 290-330 nm.

In some aspects, the rare earth element is Dy, wherein a fluorescence emission at 550-590 nm is detected upon excitation at a wavelength of less than 400 nm, and in certain aspects, between 290-330 nm.

In some aspects, the solid test mixture is substantially free of a solvent.

The foregoing paragraphs have been provided by way of general introduction, and are not intended to limit the scope of any claims. The described embodiments, together with further advantages, will be best understood by reference to the following detailed description in conjunction with the accompanying drawings.

### BRIEF DESCRIPTION OF THE DRAWINGS

FIG. 1A is an exemplary scheme illustrating a fluorescence-based system for REEs in accordance with embodiments of the present technology.

FIG. 1B illustrates REE and actinide aqueous samples containing a light harvesting molecule, pictured under UV light. Similar samples without the light harvesting molecule do not fluoresce e under the same conditions.

FIGS. 2A-2F include REEs and a light-harvesting molecule. FIG. 2A is a picture showing Dy interacting with a light-harvesting molecule under UV light. FIG. 2B is a plot showing coarse estimation of detection limit of Dy with the light-harvesting molecule. FIG. 2C is a picture showing Eu interacting with a light-harvesting molecule under UV light. FIG. 2D is a plot showing coarse estimation of detection limit of Eu with the light-harvesting molecule. FIG. 2E is a picture showing Tb interacting with a light-harvesting molecule under UV light. FIG. 2F is a plot showing coarse estimation of detection limit of Tb with the light-harvesting molecule.

FIGS. 3A-3E include Tb(III) and ciclopirox. FIG. 3A is a picture showing a mixture containing about 1 mM Tb(III) and ciclopirox under UV light. FIG. 3B is a plot showing excitation spectrum of the mixture containing about 25 UM Tb(III) and ciclopirox.

FIG. 3C is a plot showing fluorescence emission spectrum of the mixture containing about 25 μM Tb(III) and ciclopirox. FIG. 3D is a plot showing fluorescence decay trace of the mixture containing about 25 UM Tb(III) and ciclopirox. FIG. 3E is a plot showing fluorescence decay residuals of the mixture containing about 25 μM Tb(III) and ciclopirox.

FIGS. 4A-4E include Eu(III) and ciclopirox. FIG. 4A is a picture showing a mixture containing about 1 mM Eu(III) and ciclopirox under UV light. FIG. 4B is a plot showing excitation spectrum of the mixture containing about 25 μM Eu(III) and ciclopirox.

FIG. 4C is a plot showing fluorescence emission spectrum of the mixture containing about 25 UM Eu(III) and ciclopirox. FIG. 4D is a plot showing fluorescence decay trace of the mixture containing about 25 UM Eu(III) and ciclopirox. FIG. 4E is a plot showing fluorescence decay residuals of the mixture containing about 25 UM Eu(III) and ciclopirox.

FIGS. 5A-5C include Dy(III) and ciclopirox. FIG. 5A is a picture showing a mixture containing about 1 mM Dy(III) and ciclopirox under UV light. FIG. 5B is a plot showing excitation spectrum of the mixture containing about 25 μM Dy(III) and ciclopirox. FIG. 5C is a plot showing fluorescence emission spectrum of the mixture containing about 25 UM Dy(III) and ciclopirox.

FIGS. 6A-6C include Sm(III) and ciclopirox. FIG. 6A is a picture showing a mixture containing about 25 μM Sm(III) and ciclopirox under UV light. FIG. 6B is a plot showing excitation spectrum of the mixture containing about 25 μM Sm(III) and ciclopirox. FIG. 6C is a plot showing fluorescence emission spectrum of the mixture containing about 25 μM Sm(III) and ciclopirox.

FIGS. 7A-7C show certain detection limits of ciclopirox. FIG. 7A are plots showing determination of detection limit of Tb(III) using ciclopirox. FIG. 7B are plots showing determination of detection limit of Eu(III) using ciclopirox. FIG. 7C are plots showing determination of detection limit of Dy(III) using ciclopirox.

FIGS. 8A-8C show certain fluorescence emission spectrums of ciclopirox. FIG. 8A is a plot showing fluorescence emission spectrum of the mixture containing Pr(III) and ciclopirox. FIG. 8B is a plot showing fluorescence emission spectrum of the mixture containing Ho(III) and ciclopirox. FIG. 8C is a plot showing fluorescence emission spectrum of the mixture containing about 6 μM Am(III) and about 40 μM ciclopirox.

FIG. 9 is a picture showing a mixture of Eu(III) and ciclopirox in water (left), and a mixture of Eu(III) in water (control sample, right), both of which are under UV light (254 nm) with ambient light off.

FIG. 10 is a picture showing a mixture of Tb(III) and ciclopirox in water (left), and a mixture of Tb(III) in water (control sample, right), both of which are under UV light (254 nm) with ambient light off.

FIGS. 11A-11B show certain mixtures of Tb(III) and ciclopirox. FIG. 11A is a picture showing a mixture of Tb(III) and ciclopirox in water (left), and a mixture of Tb(III) in water (control sample, right), both of which are under UV light (254 nm) with ambient light on. FIG. 11B is a picture showing a series of mixtures containing different concentrations of Tb(III) and ciclopirox in water under UV light (254 nm) with ambient light off. From left to right, the first sample on the left does not contain ciclopirox, followed by 0.10, 0.15, 0.20, 0.25, 0.30, 0.50, and 1.0 equivalent of ciclopirox relative to Tb(III).

FIGS. 12A-12B show certain solid precipitations of complexes of ciclopirox, confirming that the luminescence properties are retained in the solid state, even for amorphous precipitates. FIG. 12A is a picture showing solid precipitations of complexes of ciclopirox with REEs (from left to right, Ce(IV) (non-luminescent control), Nd(III) (non-luminescent control), Sm(III), Eu(III), and (Tb(III)) under ambient light. FIG. 12B is a picture showing solid precipitations of complexes of ciclopirox with REEs (from left to right, Ce(IV) (non-luminescent control), Nd(III) (non-luminescent control), Sm(III), Eu(III), and (Tb(III)) under UV light.

FIGS. 13A-13D show certain mixtures of light-harvesting molecules in water. FIG. 13A is a picture showing a mixture of about 100 μM Eu(III) and about 400 μM piroctone in water at a pH of 6 (left) and a mixture of about 100 μM Eu(III) and about 400 μM ciclopirox in water at a pH of 6 (right), both of which are under UV light with ambient light off. FIG. 13B is a picture showing mixtures of about 100 μM Tb(III) and about 400 μM ciclopirox in water at a pH of 6 under UV light with ambient light off. FIG. 13C is a picture showing mixtures of about 100 μM Tb(III) and about 400 μM piroctone in water at a pH of 6 under UV light with ambient light off. FIG. 13D is a picture showing a mixture of about 100 μM Dy(III) and about 400 μM ciclopirox in water at a pH of 6 (left) and a mixture of about 100 μM Dy(III) and about 400 μM piroctone in water at a pH of 6 (right), both of which are under UV light with ambient light off.

FIGS. 14A-14C show certain mixtures of light-harvesting molecules in water or 70% ethanol. FIG. 14A is a picture showing a mixture of about 75 μM Dy(III) and piroctone in water contained in a plastic cuvette (left), and a mixture of about 75 μM Eu(III) and piroctone in water contained in a plastic cuvette (left), both of which are under UV light with ambient light off. FIG. 14B is a picture showing a mixture of Tb(III) and ciclopirox in 70% ethanol contained in a glass tube (left), a mixture of Eu(III) and ciclopirox in 70% ethanol contained in a glass tube (middle), and a mixture of Dy(III) and ciclopirox in 70% ethanol contained in a glass tube (right), all of which are under UV light with ambient light off. FIG. 14C is a picture showing, from left to right, a first negative control in 70% ethanol contained in a glass tube, a second negative control in 70% ethanol contained in a glass tube, a mixture of Eu(III) and piroctone in 70% ethanol contained in a glass tube, and a mixture of Tb(III) and piroctone in 70% ethanol contained in a glass tube (right), all of which are under UV light with ambient light off. Containers made of regular glass are likely to absorb most of the incident UV light, hence decreasing the apparent brightness of the samples.

FIGS. 15A-15D show certain mixtures of piroctone in 70% ethanol (all in a quartz cuvette). FIG. 15A is a picture showing a mixture of about 0.4 mM Tb(III) and piroctone in 70% ethanol under UV light (254 nm) with ambient light off. FIG. 15B is a picture showing a mixture of about 0.4 mM Eu(III) and piroctone in 70% ethanol under UV light (254 nm) with ambient light off. FIG. 15C is a picture showing a mixture of about 0.4 mM Dy(III) and piroctone in 70% ethanol under UV light (254 nm) with ambient light off. FIG. 15D is a picture showing a mixture of about 0.4 mM Sm(III) and piroctone in 70% ethanol under UV light (254 nm) with ambient light off.

FIGS. 16A-16C show certain mixtures of piroctone in 70% ethanol (all in a quartz cuvette). FIG. 16A is a picture showing a mixture of about 0.4 mM Tb(III) and piroctone in 70% ethanol under UV light (254 nm) with ambient light off. FIG. 16B is a picture showing a mixture of about 0.4 mM Tb(III) and piroctone in 70% ethanol under UV light (365 nm) with ambient light off. FIG. 16C is a picture showing a mixture of about 0.4 mM Tb(III) and piroctone in 70% ethanol under UV light (254 nm) with ambient light on.

FIGS. 17A-17D show certain properties of aqueous solutions containing 25 μM Dy(III) and ciclopirox. FIG. 17A is a plot showing excitation spectrum of an aqueous solution containing Dy(III) and ciclopirox. FIG. 17B is a plot showing fluorescence emission spectrum of the aqueous solution containing Dy(III) and ciclopirox. FIG. 17C is a plot showing emission map of the aqueous solution containing Dy(III) and ciclopirox at different excitation wavelengths. FIG. 17D is a plot showing time-resolved fluorescence decay of the aqueous solution containing Dy(III) and ciclopirox.

FIGS. 18A-18D show certain properties of aqueous solutions containing 25 μM Eu(III) and ciclopirox. FIG. 18A is a plot showing excitation spectrum of an aqueous solution containing Eu(III) and ciclopirox. FIG. 18B is a plot showing fluorescence emission spectrum of the aqueous solution containing Eu(III) and ciclopirox. FIG. 18C is a plot showing emission map of the aqueous solution containing Eu(III) and ciclopirox at different excitation wavelengths. FIG. 18D is a plot showing time-resolved fluorescence decay of the aqueous solution containing Eu(III) and ciclopirox.

FIGS. 19A-19D show certain properties of aqueous solutions containing 25 μM Sm(III) and ciclopirox. FIG. 19A is a plot showing excitation spectrum of an aqueous solution containing Sm(III) and ciclopirox. FIG. 19B is a plot showing fluorescence emission spectrum of the aqueous solution containing Sm(III) and ciclopirox. FIG. 19C is a plot showing emission map of the aqueous solution containing Sm(III) and ciclopirox at different excitation wavelengths. FIG. 19D is a plot showing time-resolved fluorescence decay of the aqueous solution containing Sm(III) and ciclopirox.

FIGS. 20A-20D show certain properties of aqueous solutions containing 25 μM Tb(III) and ciclopirox. FIG. 20A is a plot showing excitation spectrum of an aqueous solution containing Tb(III) and ciclopirox. FIG. 20B is a plot showing fluorescence emission spectrum of the aqueous solution containing Tb(III) and ciclopirox. FIG. 20C is a plot showing emission map of the aqueous solution containing Tb(III) and ciclopirox at different excitation wavelengths. FIG. 20D is a plot showing time-resolved fluorescence decay of the aqueous solution containing Tb(III) and ciclopirox.

FIGS. 21A-21B show certain properties of aqueous solutions containing 25 μM Pr(III) and ciclopirox. FIG. 21A is a plot showing excitation spectrum of an aqueous solution containing Pr(III) and ciclopirox. FIG. 21B is a plot showing fluorescence emission spectrum of the aqueous solution containing Pr(III) and ciclopirox.

FIGS. 22A-22B show certain properties of aqueous solutions containing 25 μM Ho (III) and ciclopirox. FIG. 22A is a plot showing excitation spectrum of an aqueous solution containing Ho(III) and ciclopirox FIG. 22B is a plot showing fluorescence emission spectrum of the aqueous solution containing Ho(III) and ciclopirox.

FIGS. 23A-23C show certain properties of solutions containing a REE and ciclopirox. FIG. 23A are plots showing estimation of detection limit of Tb(III) using ciclopirox in water at a pH of 7. FIG. 23B are plots showing estimation of detection limit of Eu(III) using ciclopirox in water at a pH of 7. FIG. 23C are plots showing estimation of detection limit of Dy(III) using ciclopirox in water at a pH of 7.

FIGS. 24A-24B show certain properties of solutions containing Am(III) and ciclopirox. FIG. 24A is a plot showing excitation spectrum of an aqueous solution containing about 6 μM Am(III) and about 40 μM ciclopirox at a pH of about 7. FIG. 24B is a plot showing fluorescence emission spectrum of the aqueous solution containing about 6 μM Am(III) and about 40 μM ciclopirox at a pH of about 7.

FIGS. 25A-25F show certain Fourier transform infrared (FT-IR) spectra overlay of mixtures comprising ciclopirox. FIG. 25A is a plot showing an overlay of Fourier-Transform Infra-Red (FT-IR) spectra of CE/ciclopirox olamine, ciclopirox, a mixture of ciclopirox and Ce(III)—Ce(IV), a mixture of ciclopirox and Eu(III), a mixture of ciclopirox and Sm(III), and piroctone olamine, respectively. FIG. 25B is a plot showing an overlay of FT-IR spectra of ciclopirox, a mixture of ciclopirox and Ce(III)—Ce(IV), and a mixture of ciclopirox and Sm(III), respectively. FIG. 25C is a plot showing an overlay of FT-IR spectra of mixtures containing ciclopirox and Ce(IV) at different ratios of Ce/ciclopirox. FIG. 25D is a plot showing an overlay of FT-IR spectra of mixtures containing ciclopirox and Ce(IV) at different ratios of Ce/ciclopirox. FIG. 25E is a plot showing an overlay of FT-IR spectra of mixtures containing ciclopirox and Er(III) at different ratios of Er/ciclopirox. FIG. 25F is a plot showing an overlay of FT-IR spectra of mixtures containing ciclopirox and Nd(III) at different ratios of Er/ciclopirox.

FIGS. 26A-26B show certain proton magnetic resonance (1H NMR) spectrum of ciclopirox. FIG. 26A shows 1H NMR spectrum of a mixture of Ce(IV) and ciclopirox. FIG. 26B shows a 1H NMR spectrum of ciclopirox.

FIGS. 27A-27F show certain oxidation data of ciclopirox. FIG. 27A is a picture showing spontaneous oxidation of Ce(III) (left, uncolored) to Ce(IV) (right, orange) by ciclopirox. FIG. 27B is a picture showing spontaneous oxidation of Ce(III) (left, uncolored) to Ce(IV) (right, orange) by piroctone. FIG. 27C is a picture showing a Ce(IV)-ciclopirox mixture stable in the solid state. FIG. 27D is a plot showing the kinetics of oxidation of Ce(III) to Ce(IV) by ciclopirox monitored by UV-vis spectrophotometry. FIG. 27E is a plot calculating the kinetics of oxidation of Ce(III) to Ce(IV) by ciclopirox. FIG. 27F is a plot showing an overlay of FT-IR spectra of mixtures containing Ce(III)/Ce(IV)-ciclopirox, Sm(III)-ciclopirox, and unbound ciclopirox.

FIGS. 28A-28B show certain mixtures of REEs and ciclopirox under UV light. FIG. 28A is a picture showing, from left to right, a mixture of Sm(III) and ciclopirox, a mixture of Dy(III) and ciclopirox, a mixture of Eu(III) and ciclopirox, and a mixture of Tb(III) and ciclopirox, under UV light (254 nm). FIG. 28B is a picture showing a mixture of Tb(III) and piroctone under UV light (254 nm).

FIGS. 29A-29B show certain features of Nd(III) and ciclopirox. FIG. 29A is a plot showing complexation of Nd(III) by ciclopirox using UV-vis spectroscopy. FIG. 29B is a plot estimating complex stoichiometry of Nd(III) and ciclopirox.

FIGS. 30A-30H show certain features of mixtures comprising REEs and ciclopirox in water. FIG. 30A is a plot showing fluorescence emission spectrum of a mixture containing about 5 μM Eu(III) and about 20 μM ciclopirox in water at a pH of 7. FIG. 30B is a plot summarizing fluorescence emission peak areas of a mixture containing Eu(III) and ciclopirox measured as a function of pH. FIG. 30C is a plot showing fluorescence emission spectrum of a mixture containing about 5 μM Dy(III) and about 20 μM ciclopirox in water at a pH of 7. FIG. 30D is a plot summarizing fluorescence emission peak areas of a mixture containing Dy(III) and ciclopirox measured as a function of pH. FIG. 30E is a plot showing fluorescence emission spectrum of a mixture containing about 5 μM Sm(III) and about 20 μM ciclopirox in water at a pH of 7. FIG. 30F is a plot summarizing fluorescence emission peak areas of a mixture containing Sm(III) and ciclopirox measured as a function of pH. FIG. 30G is a plot showing fluorescence emission spectrum of a mixture containing about 5 μM Tb(III) and ciclopirox in water at a pH of 7. FIG. 30H is a plot summarizing fluorescence emission peak areas of a mixture containing Tb(III) and ciclopirox measured as a function of pH.

FIGS. 31A-31H show certain features of mixtures comprising REEs and piroctone in water. FIG. 31A is a plot showing fluorescence emission spectrum of a mixture containing Eu(III) and piroctone in water at a pH of 7. FIG. 31B is a plot summarizing fluorescence emission peak areas of a mixture containing Eu(III) and piroctone measured as a function of pH. FIG. 31C is a plot showing fluorescence emission spectrum of a mixture containing Dy(III) and piroctone in water at a pH of 7. FIG. 31D is a plot summarizing fluorescence emission peak areas of a mixture containing Dy(III) and piroctone measured as a function of pH. FIG. 31E is a plot showing fluorescence emission spectrum of a mixture containing Sm(III) and piroctone in water at a pH of 7. FIG. 31F is a plot summarizing fluorescence emission peak areas of a mixture containing Sm(III) and piroctone measured as a function of pH. FIG. 31G is a plot showing fluorescence emission spectrum of a mixture containing Tb(III) and piroctone in water at a pH of 7. FIG. 31H is a plot summarizing fluorescence emission peak areas of a mixture containing Tb(III) and piroctone measured as a function of pH.

FIGS. 32A-32B show certain features of mixtures comprising REEs and ciclopirox or piroctone. FIG. 32A is a plot showing an overlay of fluorescence emission spectra of Tb(III) in the presence of ciclopirox and piroctone, respectively. FIG. 32B is a plot showing an overlay of fluorescence emission spectra of Eu(III) in the presence of ciclopirox and piroctone, respectively.

FIG. 33 is a picture showing a mixture of Eu(III) and ciclopirox, which was deposited as an ethanol solution on paper and then dried in air (left), and a mixture of Tb(III) and ciclopirox, which was deposited as an ethanol solution on paper and then dried in air (right). Picture taken with UV light on.

FIGS. 34A-34B show certain features of mixtures comprising REEs and ciclopirox. FIG. 34A is a plot showing fluorescence emission spectrum of a mixture of Eu(III) and ciclopirox in solid state. FIG. 34B is a plot showing fluorescence emission spectrum of a mixture of Tb(III) and ciclopirox in solid state.

FIGS. 35A-35B show certain features of mixtures comprising Eu(III) and ciclopirox. FIG. 35A is a picture of solid precipitate of Eu(III) and ciclopirox under ambient light. FIG. 35B is a picture of solid precipitate of Eu(III) and ciclopirox under UV light with ambient light on.

FIGS. 36A-36B show certain features of mixtures comprising Cm(III) and ciclopirox. FIG. 36A is a picture showing a mixture of Cm(III) and piroctone under UV light (365 nm) with ambient light off. FIG. 36B is a picture showing a mixture of Cm(III) and ciclopirox under UV light (365 nm) with ambient light off.

FIGS. 37A-37B show certain features of mixtures comprising Cm(III) and ciclopirox or piroctone. FIG. 37A is a plot showing fluorescence emission spectrum of a mixture of Cm(III) and ciclopirox. FIG. 37B is a plot showing fluorescence emission spectrum of a mixture of Cm(III) and piroctone.

FIGS. 38A-38C show certain features of mixtures comprising Cm(III) and ciclopirox. FIG. 38A is a plot showing an overlay of fluorescence emission spectra of mixtures of about 20 μM ciclopirox and different concentrations of Cm(III) (5E-10 to 1E-6 M) in water at a pH of 7 for estimation of detection limit. FIG. 38B is a plot showing an overlay of fluorescence emission spectra of mixtures of ciclopirox and different concentrations of Cm(III) (0, 5E-10, 1E-9, and 5E-9 mol/L). FIG. 38C is a plot showing estimation of detection limit and linearity range of Cm(III) luminescence using ciclopirox in water at a pH of 7.

FIGS. 39A-39B show certain features of mixtures comprising Tb(III) and ciclopirox. FIG. 39A is a plot showing an overlay of fluorescence emission spectra of mixtures of ciclopirox and Tb(III) in different solvents. FIG. 39B is a bar graph summarizing emission intensities at 546 nm of mixtures of ciclopirox and Tb(III) in different solvents.

FIGS. 40A-40D show certain features of a light-harvesting molecules. FIG. 40A illustrates chemical structures of ciclopirox and piroctone. FIG. 40B illustrates chemical structures of SUN-B, tenellin, and pyridoxatin. FIG. 40C is a plot showing an overlay of fluorescence emission spectra of mixtures containing about 5 μM Eu(III) and about 30 μM piroctone, about 5 μM Eu(III) and about 30 μM ciclopirox, about 5 μM Eu(III) and about 30 μM pyridoxatin, about 5 μM Eu(III) and about 30 μM SUN-B, and about 5 μM Eu(III) and about 30 μM tenellin, respectively. FIG. 40D is a plot showing an overlay of fluorescence emission spectra of mixtures containing about 5 μM Tb(III) and about 30 μM piroctone, about 5 μM Tb(III) and about 30 μM ciclopirox, about 5 μM Tb(III) and about 30 μM pyridoxatin, about 5 μM Tb(III) and about 30 μM SUN-B, and about 5 μM Tb(III) and about 30 μM tenellin, respectively.

### DETAILED DESCRIPTION

### Definitions

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a,” “an,” and “the” include plural referents unless the context clearly dictates otherwise. Thus, for example, reference to “a light-harvesting molecule” includes a plurality of light-harvesting molecules.

Ranges recited herein are intended as continuous ranges, including every value between the minimum and maximum values recited, as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein, and in all instances such ratios, ranges, and ranges of ratios represent various embodiments of the present technology.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the terms “molecule”, “complex”, “compound”, and “product” are used interchangeably, and are intended to refer to a chemical entity, whether in the solid, liquid, or gaseous phase, and whether in a crude mixture or purified and isolated.

As used herein, “salt” refers to derivatives of the disclosed compounds or chemical structures wherein the parent complex is modified by making acid or base salts thereof.

As used herein, the term “solvate” refers to a physical association of a compound of this disclosure with one or more solvent molecules, whether organic or inorganic.

Compounds or chemical structures of the present technology, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure.

Throughout the specification and the appended claims, a given chemical formula or structure shall encompass all stereo and optical isomers and racemates thereof where such isomers exist. Unless otherwise indicated, all chiral (enantiomeric and diastereomeric) and racemic forms are within the scope of the disclosure. Many geometric isomers of C═N double bonds, ring systems, and the like can also be present in the complexes, and all such stable isomers are contemplated in the present disclosure.

The term “alkyl” describes an aliphatic hydrocarbon including straight chain and branched chain groups.

The term “alkylene” as used herein refers to a divalent form of an alkane with a general formula of CₙH₂ₙ, wherein n may range from 2 to 12, or 3 to 8.

The term “alkenyl” refers to a straight, or branched hydrocarbon fragment containing at least one C═C double bond.

The term “alkynyl” refers to unsaturated hydrocarbon fragment having at least one C═C triple bond.

The term “cycloalkyl” refers to cyclized alkyl groups.

The term “cycloalkenyl” refers to a cyclic hydrocarbon fragment containing at least one C═C double bond.

The term “aryl” means a carbocyclic aromatic monocyclic group containing 6 carbon atoms which may be further fused to a second 5- or 6-membered carbocyclic group which may be aromatic, saturated, or unsaturated.

The term “amide” refers to unsubstituted amide (i.e., —CONH₂), or substituted amide (e.g., —CONHalkyl, —CONHaryl, —CONarylalkyl).

As used herein, the term “substituted” refers to at least one hydrogen atom that is replaced with a non-hydrogen group, provided that normal valencies are maintained and that the substitution results in a stable compound. When a group is noted as “optionally substituted”, the group may or may not contain non-hydrogen substituents.

As used herein, the term “luminescence” refers to spontaneous emission of light by a substance not resulting from heat. Non-limiting examples of luminescence include electroluminescence, chemiluminescence, mechanoluminescence, and photoluminescence such as fluorescence and phosphorescence.

As used herein, the term “fluorescence” refers to a process where a material absorbs light at high energy, short wavelength, and emits light at lower energy, usually visible, wavelength.

### Compositions Comprising Light-Harvesting Molecules

Aspects of the disclosure provide compositions comprising buffers and light-harvesting molecules, which can produce luminescence (e.g., fluorescence) upon interaction with rare earth elements and/or actinide elements. These light-harvesting molecules contain a hydroxypyridinone (HOPO) group. In some embodiments, the HOPO group is a 1,2-hydroxypyridinone (1,2-HOPO) or a 2,1-hydroxypyridinone (2,1-HOPO) group. The generic structure of 1,2-hydroxypyridinone (HOPO) group is shown below:

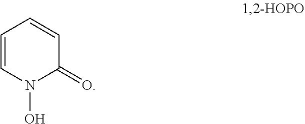

In one aspect, the composition disclosed herein comprises a light-harvesting molecule capable of producing luminescence upon interaction with a rare earth element and/or an actinide element, the light-harvesting molecule comprising one hydroxypyridinone (HOPO) group having the structure of formula (I) or (II):

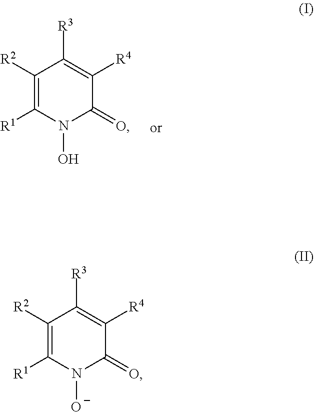

or a salt, solvate, tautomer, or stereoisomer thereof,
wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some embodiments, the light-harvesting molecule comprises one hydroxypyridinone (HOPO) group having the structure of formula (I), or a salt, solvate, tautomer, or stereoisomer thereof.

In some embodiments, the light-harvesting molecule comprises one hydroxypyridinone (HOPO) group having the structure of formula (II), or a salt, solvate, tautomer, or stereoisomer thereof.

In some embodiments, the light-harvesting molecule lacks an amide group. The amide group may be unsubstituted or substituted amide.

In terms of R¹-R⁴, these substituents may be the same or different. In some embodiments, each of R¹-R⁴ are independently selected from a hydrogen, a hydroxy group, or a hydrocarbon group, wherein the hydrocarbon group is an alkyl group, a substituted alkyl group, a cycloalkyl group, or a substituted cycloalkyl group. wherein R¹ is —CH₂CH(CH₃) CH₂C(CH₃)₃, cyclohexyl, or hydrogen, R² is hydrogen, R³ is a methyl group (—CH₃) or a hydroxy group, and R⁴ is hydrogen or

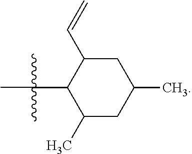 [SMILES: *C1C(C)CC(C)CC1C=C]

In some embodiments, R¹ is a hydrocarbon group having from 1 to 20 carbon atoms, wherein the hydrocarbon group is an alkyl group, an alkylene group, an alkenyl group, an alkynyl group, a substituted alkyl group, a substituted alkylene group, a substituted alkenyl group, a substituted alkynyl group, a cycloalkyl group, a cycloalkenyl group, an aryl group, a substituted cycloalkyl group, a substituted cycloalkenyl group, or a substituted aryl group.

In some embodiments, R¹ is an alkyl group that is straight or branched. In some embodiments, R¹ is an alkyl group having between 1 to 20 carbon atoms, between 2 to 18 carbon atoms, between 3 to 16 carbon atoms, between 4 to 14 carbon atoms, between 5 to 12 carbon atoms, between 6 to 10 carbon atoms, or 8 carbon atoms. In some embodiments, R¹ is an alkyl group having between 1 to 10 carbon atoms. In some embodiments, R¹ is an alkyl group having 8 carbon atoms. In some embodiments, R¹ is —CH₂CH(CH₃)CH₂C(CH₃)₃.

In some embodiments, R¹ is an alkyl group that is cyclized. In some embodiment, R¹ is a cycloalkyl group having between 3 to 14 carbon atoms, between 4 to 10 carbon atoms, between 5 to 8 carbon atoms, or between 6 to 7 carbon atoms. In some embodiment, R¹ is a substituted cycloalkyl group having between 4 and 18 carbon atoms, between 5 to 16 carbon atoms, between 6 to 12 carbon atoms, between 7 to 10 carbon atoms, or between 8 to 9 carbon atoms. In some embodiment, R¹ is a cycloalkyl or substituted cycloalkyl group having between 3 and 8 carbon atoms. In some embodiments, R¹ is a cycloalkyl or substituted cycloalkyl group having 6 carbon atoms. In some embodiments, R¹ is cyclohexyl.

In some embodiments, R¹ is hydrogen.

In some embodiments, R² is hydrogen.

In some embodiments, R³ is an alkyl group that is straight or branched. In some embodiments, R³ is an alkyl group having between 1 to 12 carbon atoms, between 2 to 10 carbon atoms, between 3 to 8 carbon atoms, between 4 to 6 carbon atoms, or 5 carbon atoms. In some embodiments, R³ is an alkyl group having from 1 to 6 carbon atoms. In some embodiments, R³ is a methyl group (—CH₃).

In some embodiments, R³ is a hydroxy group.

In some embodiments, R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

In some embodiments, R⁴ is an alkyl group that is cyclized. In some embodiment, R⁴ is a cycloalkyl group having between 3 to 14 carbon atoms, between 4 to 10 carbon atoms, between 5 to 8 carbon atoms, or between 6 to 7 carbon atoms. In some embodiment, R⁴ is a substituted cycloalkyl group having between 4 and 22 carbon atoms, between 5 to 20 carbon atoms, between 6 to 18 carbon atoms, between 7 to 16 carbon atoms, between 8 to 14 carbon atoms, between 9 to 12 carbon atoms, or between 10 to 11 carbon atoms. In some embodiments, R⁴ is a cycloalkyl or substituted cycloalkyl group having between 5 and 14 carbon atoms. In some embodiments, R⁴ is a cycloalkyl or substituted cycloalkyl group having 10 carbon atoms. In some embodiments, R⁴ is

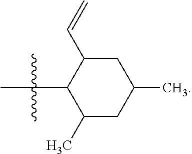 [SMILES: *C1C(C)CC(C)CC1C=C]

In some embodiments, R⁴ is hydrogen.

In some embodiments, the light-harvesting molecule comprises one hydroxypyridinone (HOPO) group having the structure of formula (II), or a salt, solvate, tautomer, or stereoisomer thereof. In some embodiments, (i) R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms, such as an alkyl group having between 1 to 10 carbon atoms, or an alkyl group having 8 carbon atoms (e.g., —CH₂CH(CH₃)CH₂C(CH₃)₃); (ii) R² is hydrogen; (iii) R³ is an alkyl group having from 1 to 6 carbon atoms, such as a methyl group; and (iv) R⁴ is hydrogen.

In some embodiments, the light-harvesting molecule comprises the structure of piroctone or a salt thereof:

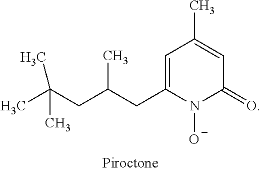 [SMILES: Cc1cc(CC(C)CC(C)(C)C)n([O-])c(=O)c1]

In some embodiments, piroctone is in a salt form. In some embodiments, piroctone is in the form of piroctone olamine. Other brand names for piroctone olamine include, but are not limited to, piroctone ethanolamine, Octopirox®, octopyrox, and kopirox.

In some embodiments, the light-harvesting molecule comprises one hydroxypyridinone (HOPO) group having the structure of formula (I), or a salt, solvate, tautomer, or stereoisomer thereof. In some embodiments, (i) R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms, such as a cycloalkyl or substituted cycloalkyl group having between 3 and 8 carbon atoms, or a cycloalkyl or substituted cycloalkyl group having 6 carbon atoms (e.g., cyclohexyl); (ii) R² is hydrogen; (iii) R³ is an alkyl group having from 1 to 6 carbon atoms, such as a methyl group; and (iv) R⁴ is hydrogen.

In some embodiments, the light-harvesting molecule comprises the structure of ciclopirox or a salt thereof:

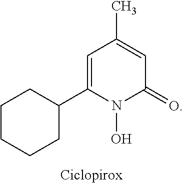

Other brand names for ciclopirox include, but are not limited to, penlac, loprox, and HOE 296b.

In some embodiments, ciclopirox is in a salt form. In some embodiments, ciclopirox is in the form of ciclopirox olamine (i.e., ciclopirox ethanolamine).

In some embodiments, the light-harvesting molecule comprises one hydroxypyridinone (HOPO) group having the structure of formula (I), or a salt, solvate, tautomer, or stereoisomer thereof. In some embodiments, (i) R¹ is hydrogen; (ii) R² is hydrogen; (iii) R³ is a hydroxy group; and (iv) R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms, such as a cycloalkyl or substituted cycloalkyl group having between 5 and 14 carbon atoms, or a cycloalkyl or substituted cycloalkyl group having 10 carbon atoms (e.g.,

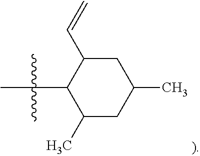 [SMILES: *C1C(C)CC(C)CC1C=C]

In some embodiments, the light-harvesting molecule comprises the structure of pyridoxatin, or an enantiomer, or a tautomer thereof:

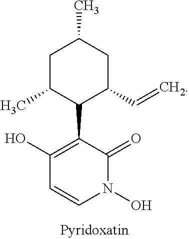

In some embodiments, the light-harvesting molecule comprises a tautomer of pyridoxatin.

In another aspect, the composition disclosed herein comprises a buffer. The buffer may an aqueous solution comprising a buffering agent. Non-limiting examples of buffer agent include acetic acid, citric acid, 4-(2-hydroxyethyl)-1-piperazineethanesulfonic acid (HEPES), tris(hydroxymethyl)aminomethane (TRIS), piperazine-N,N′-bis(2-ethanesulfonic acid) (PIPES), 2-(N-morpholino)ethanesulfonic acid (MES), N-cyclohexyl-2-aminoethanesulfonic acid (CHES), carbonate/bicarbonate mixtures, phosphate buffers such as monosodium phosphate, disodium phosphate, sodium tripolyphosphate buffers, and phosphate/hydrogenoephosphate mixtures, and borate buffers.

In some embodiments, the composition is in the form of an aqueous solution. In some embodiments, the composition is in the form of a solution, which further comprises water and/or an organic solvent.

The water may include fresh water (e.g., tap water, distilled water, bidistilled water, deionized water, deionized distilled water, reverse osmosis water, well water, or fresh water obtained from natural sources such as lakes, groundwaters, streams, rivers, etc.) or salt water such as seawater.

Exemplary organic solvents that may be used in the technology disclosed herein include, but are not limited to, formamides/acetamides (e.g., formamide, dimethylformamide (DMF), dimethyl acetamide), dimethyl sulfoxide (DMSO), acetonitrile, alkane solvents (e.g., pentane, cyclopentane, hexanes, cyclohexane, heptanes, cycloheptane, octanes), ethers (e.g., diethyl ether, tetrahydrofuran (THF), 1,4-dioxane), glycol ethers (e.g., diglyme, triglyme), ester solvents (e.g., ethyl acetate), and ketones (e.g., acetone, butanone).

In some embodiments, the organic solvent is at least one selected from the group consisting of an alcohol, acetonitrile, dimethylformamide (DMF), and dimethyl sulfoxide (DMSO). Exemplary alcohols include, but are not limited to, monoalcohols (e.g., methanol, ethanol, propanol, isopropanol, n-butanol, isobutanol), polyalcohols including glycols. In some embodiments, the alcohol is ethanol.

The composition may comprise the light-harvesting molecule disclosed herein in any of its embodiments and a buffer. In some embodiments, the light-harvesting molecule is present in an amount of about 0.01 μM-100 mM, about 0.1 μM-50 mM, about 0.5 M-10 mM, about 1 μM-1 mM, about 5 M-800 M, about 10 μM-600 μM, about 20 μM-500 μM, about 30 μM-400 μM, about 40 μM-300 μM, about 50 μM-200 μM, or about 75 μM-100 μM, relative to a total volume of the composition.

In some embodiments, the composition has a pH between about 5 to about 11.5, between about 6 to about 11, between about 7 to about 10, between about 8 to about 9. In some embodiments, the composition has a mild acidic pH of 5, 5.5, 6, or 6.5. In some embodiments, the composition has a neutral pH of about 7. In some embodiments, the composition has a basic pH of 7.5, 8, 8.5, 9, 9.5, 10, 10.5, 11, or 11.5.

The light-harvesting molecule can bind any of rare earth elements including lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc), yttrium (Y), or any combinations thereof. The light-harvesting molecule can bind any of the rare earth elements in any oxidation state (e.g., Eu²⁺, Sm³⁺, Ln²⁺, Ln³⁺, Ln⁴⁺, etc.). In some embodiments, the light-harvesting molecule binds the rare earth elements in +III oxidation state and/or +IV oxidation state.

In some embodiments, the light-harvesting molecule is capable of producing luminescence upon interaction with at least one rare earth element selected from the group consisting of Sm³⁺, Eu³⁺, Tb³⁺, Dy³⁺, Pr³⁺, and Ho³⁺.

The light-harvesting molecule can bind any of actinide elements including, but not limited to, uranium (U), thorium (Th), plutonium (Pu), americium (Am), actinium (Ac), neptunium (Np), and curium (Cm). The light-harvesting molecule can bind any of the actinide elements in any oxidation state (e.g., Cm³⁺, Pu⁴⁺, Bk⁴⁺, NpO²⁺, UO₂²⁺, etc.). In certain embodiments, the actinide element is not protactinium (Pa).

In some embodiments, the light-harvesting molecule is capable of producing luminescence upon interaction with an actinide element which is Cm and/or Am.

In some embodiments, two light-harvesting molecules bind one rare earth element or one actinide element. For example, in some embodiments, the stochiometric ratio of rare earth element to molecule is 1:2. In some embodiments, the light-harvesting molecule and the rare earth element form a complex which comprises two molecules per one rare earth element. In some embodiments, the stochiometric ratio of actinide element to molecule is 1:2. In some embodiments, the light-harvesting molecule and the actinide element form a complex which comprises two light-harvesting molecules per one actinide element.

### Assay Systems and Kits

Also provided herein are assay systems comprising compositions that contain the light-harvesting molecules capable of producing luminescence upon interaction with rare earth elements and/or actinide elements.

In some embodiments, the assay system comprises one or more of the aforementioned composition (i.e., compositions comprising the buffer and the light-harvesting molecule).

In some embodiments, the assay system comprises the light-harvesting molecule disclosed herein in any of its embodiments immobilized on an assay test strip.

The assay test strip refers to a substrate material to which the light-harvesting molecule is immobilized using methods known to a person of ordinary skill in the art. A variety of materials can be used as the substrate, including any material that can act as a support for attachment of the light-harvesting molecule. Exemplary materials include, but are not limited to, silicon, glass, paper, wood, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone (PVP), rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamide, polyacrylamide blends, co-polymers of vinyl and acrylamide, methacrylates, methacrylate derivatives and co-polymers, other polymers and co-polymers with various functional groups, latex, butyl rubber and other synthetic rubbers, natural sponges, insoluble protein, surfactants, red blood cells, metals, metalloids, magnetic materials, or other commercially available media or a complex material composed of a solid or semi-solid substrate coated with materials that improve the hydrophilic property of the strip substrate, for example, polystyrene, Mylar, polyethylene, polycarbonate, polypropylene, polybutylene, metals such as aluminum, copper, tin or mixtures of metals coated with dextran, detergents, salts, PVP and/or treated with electrostatic or plasma discharge to add charge to the surface thus imparting a hydrophilic property to the surface.

In some embodiments, the light-harvesting molecule is present in a composition that further contains a pH buffer such as a buffering agent. In some embodiments, the composition is in the form of an aqueous solution. In some embodiments, the composition is in the form of a solution, which further comprises water and/or an organic solvent.

In some embodiments, the organic solvent is at least one selected from the group consisting of an alcohol, acetonitrile, dimethylformamide (DMF), and dimethyl sulfoxide (DMSO). Exemplary alcohols include, but are not limited to, monoalcohols (e.g., methanol, ethanol, propanol, isopropanol, n-butanol, isobutanol), polyalcohols including glycols. In some embodiments, the alcohol is ethanol.

In some embodiments, the assay test strip (e.g., substrate materials) is porous. In some embodiments, the substrate material is a fibrous material. For example, in some embodiments, the substrate material is a fibrous material produced with electrospun fibers. In some embodiments, the substrate material has a pore diameter of at least about 0.10 nm, at least about 1.0 nm, at least about 10 nm, at least about 50 nm, at least about 100 nm, at least about 150 nm, at least about 200 nm, at least about 250 nm, at least about 300 nm, at least about 350 nm, at least about 400 nm, at least about 450 nm, at least about 500 nm, at least about 550 nm, at least about 600 nm, at least about 650 nm, at least about 700 nm, at least about 750 nm, at least about 800 nm, at least about 850 nm, at least about 900 nm, at least about 950 nm, or at least about 1000 nm. In some embodiments, the substrate material has a pore diameter between about 1.0 nm to about 500 nm, about 0.10 nm to about 10 nm, about 150 nm to about 1000 nm, about 300 nm to about 600 nm, about 200 nm to about 800 nm, about 300 nm to about 500 nm, about 500 nm to about 1000 nm, or about 600 nm to about 800 nm.

Also provided herein are kits including a reagent comprising the aforementioned composition (i.e., compositions comprising the buffer and the light-harvesting molecule) and one or more containers for combining an aliquot of the reagent with a test sample.

In some embodiments, the kit includes the aforementioned assay system which comprises the light-harvesting molecule disclosed herein in any of its embodiments immobilized on the assay test strip. In some embodiments, the kit optionally includes a solution comprising a buffer for dissolving a test sample comprising a rare earth element and/or an actinide element. When the solution is present, the kit further comprises one or more containers for combining an aliquot of the solution with the test sample.

In some embodiments, the kit further comprises a light source that emits ultraviolet (UV) light with a wavelength of less than 400 nm. In some embodiments, the light source emits UV light with a wavelength of 200-400 nm, 250-350 nm, or 280-330 nm. In certain embodiments, the light source emits UV light with a wavelength of about 310 nm. In certain embodiments, the light source emits UV light with a wavelength of about 320 nm.

In some embodiments, the kit further comprises a luminescence detector that can detect a luminescent signal such as a fluorescence signal. In some embodiments, the luminescence detector can detect light in thprotactinium e visible range. In some embodiments, the fluorescence signal has a wavelength in the visible light range. In some embodiments, the fluorescence signal has a wavelength of greater than 520 nm, greater than 550 nm, greater than 600 nm, or greater than 630 nm. In some embodiments, the fluorescence signal has a wavelength of 500-700 nm, 530-670 nm, 560-620 nm, or 580-600 nm.

In some embodiments, the kit further comprises a spectrofluorometer, such as a photoluminescence reader, which detects a fluorescence signal. The fluorescence signal is excited by a light source that emits in the UV region of the optics spectrum and within the excitation band of the light harvesting molecule. The emitted fluorescence signal is detected by a photodiode and the wavelength of the signal detected may be limited using a long pass filter which blocks stray emitted light and accepts light with wavelengths at and around the peak emission wavelength of the fluorescence emitting label. In other embodiments, the long pass filter may be replaced by a band pass filter. Furthermore, the excitation light may be limited by a band pass filter. In some embodiments, the luminescence detector is a photoluminescence spectrometer. In some embodiments, the luminescence detector is in the form of a smartphone-based luminescence reader. In some embodiments, the smartphone-based reader has charge-coupled device (CCD) type cameras (CCDC) that detects photoluminescence emission in the visible range.

In some embodiments, the light source is a light emitting diode. In some embodiments, the light emitting diode is a UV laser diode. The diode may be a UV, LED, or photodiode. In some embodiments, the light emitting diode emits light having a wavelength in a range from about 210 nm to about 290 nm, from about 230 nm to about 270 nm, or about 254 nm. In some embodiments, the light emitting diode emits light having a wavelength in a range from about 290 nm to about 400 nm, from about 330 nm to about 380 nm, or about 365 nm. In some embodiments, the light emitting diode is a hand-held diode.

In some embodiments, the kit comprises (i) the aforementioned composition (i.e., compositions comprising the buffer and the light-harvesting molecule); (ii) one or more containers for combining an aliquot of the reagent with a test sample; (iii) optionally a solution; (iv) a light source that emits ultraviolet (UV) light; and (v) a luminescence detector.

The test sample may be a sample material known to contain or suspected to contain a rare earth element and/or an actinide element.

In some embodiments, the test sample contains or is suspected to contain rare earth elements including lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc), yttrium (Y), or any combinations thereof. The rare earth elements can be in any oxidation state (e.g., Eu²⁺, Sm³⁺, Ln²⁺, Ln³⁺, Ln⁴⁺, etc.).

In some embodiments, the test sample contains or is suspected to contain at least one rare earth element selected from the group consisting of Sm, Eu, Tb, Dy, Pr, and Ho.

In some embodiments, the test sample contains or is suspected to contain actinide elements including, but not limited to, uranium (U), thorium (Th), plutonium (Pu), americium (Am), actinium (Ac), protactinium (Pa), neptunium (Np), and curium (Cm). The actinide elements can be in any oxidation state (e.g., Cm³⁺, Pu⁴⁺, Bk⁴⁺, NpO²⁺, UO₂²⁺, etc.).

In some embodiments, the test sample contains or is suspected to contain an actinide element which is Cm and/or Am.

In some embodiments, the test sample material is a solid material, a semi-solid material, or an aqueous medium. In some embodiments, the test sample material is an aqueous solution. Non-limiting examples of suitable test sample material include leachates derived from rare earth ores (e.g., bastnasite, monazite, loparite, xenotime, allanite, and the lateritic ion-adsorption clays), geothermal brines, coal, coal byproducts, mine tailings, phosphogypsum, acid leachate of solid source materials, REE solution extracted from solid materials through ion-exchange methods, or other ore materials, such as REE-containing clays, volcanic ash, organic materials, and any solids/liquids that react with igneous and sedimentary rocks.

In some embodiments, the test sample is a low-grade REE material wherein the REEs are present in less than about 2 wt % of the total weight of the low-grade REE material. In other embodiments, the test sample is a high-grade REE material, wherein the REEs are present in greater than about 2 wt % of the total weight of the high-grade REE material.

In some embodiments, the test sample comprises less than about 5 wt %, less than about 10 wt %, less than about 15 wt %, less than about 20 wt %, less than about 25 wt %, less than about 30 wt %, less than about 35 wt %, less than about 40 wt %, less than about 45 wt %, or less than about 50 wt % REEs of the total weight of the test sample.

In some embodiments, the test sample is a low-grade actinide material wherein the actinides are present in less than about 2 wt % of the total weight of the low-grade actinide material. In other embodiments, the test sample is a high-grade actinide material, wherein the actinides are present in greater than about 2 wt % of the total weight of the high-grade actinide material.

In some embodiments, the test sample comprises less than about 5 wt %, less than about 10 wt %, less than about 15 wt %, less than about 20 wt %, less than about 25 wt %, less than about 30 wt %, less than about 35 wt %, less than about 40 wt %, less than about 45 wt %, or less than about 50 wt % actinides of the total weight of the test sample.

The test sample may be recycled REE-containing products including, but not limited to, compact fluorescent light bulbs, electro-ceramics, fuel cell electrodes, NiMH batteries, permanent magnets, catalytic converters, camera and telescope lenses, carbon lighting applications, computer hard drives, wind turbines, hybrid cars, x-ray and magnetic image systems, television screens, computer screens, fluid cracking catalysts, phosphor-powder from recycled lamps, and the like. These materials are characterized as containing amounts of REE, including, for example, scandium, yttrium, lanthanum, cerium, praseodymium, neodymium, samarium, promethium, europium, gadolinium, terbium, dysprosium, erbium, thulium, ytterbium, lutetium, or any combination thereof. In some embodiments, the test samples are recovered from a liquid waste stream from a given industry (e.g., an effluent from a factory that needs to be decontaminated from its REEs or hospital effluents for potential recovery of gadolinium) or a leachate solution coming from these REE containing materials.

The container of the kit may be a vessel having an internal cavity for combining an aliquot of the reagent with the test sample. The container may be cylindrical, cuboid, frustoconical, or spherical. The container walls may comprise a material including, but not limited to, glass, polypropylene, polyvinyl chloride, polyethylene, and/or polytetrafluoroethylene. The internal cavity of the container may have a volume of 0.1 mL-100 mL, 0.5 mL-80 mL, 1 mL-60 mL, 2.5 mL-50 mL, or 5 mL-25 mL. In another embodiment, for instance, for larger scale testing, the internal cavity may have a volume of 100 mL-50 L, 1 L-20 L, or2 L-10 L.

### Methods

Also provided herein are methods of detecting the presence of rare earth elements and/or actinide elements.

One aspect of the present disclosure relates to a method for detecting the presence of a rare earth element and/or an actinide element in a test sample. The method comprises: combining the test sample with the composition disclosed herein in any of its embodiments to form a test mixture; exposing the test mixture to UV light with a wavelength of less than 400 nm; and determining that the rare earth element and/or the actinide element is present in the test sample when a luminescent signal is detected by a luminescence detector.

In some embodiments, the luminescent signal is a fluorescence emission.

In some embodiments, the luminescence detector can detect a luminescent signal such as a fluorescence signal.

In some embodiments, the fluorescence signal detectable by the luminescence detector has a wavelength in the visible light range. In some embodiments, the fluorescence signal has a wavelength of greater than 520 nm, greater than 550 nm, greater than 600 nm, or greater than 630 nm. In some embodiments, the fluorescence signal has a wavelength of 500-700 nm, 530-670 nm, 560-620 nm, or 580-600 nm. Such feature enables easy observation and quantification of the detection technology disclosed herein.

In some embodiments, the luminescent signal (e.g., fluorescence emission) is detected by a photoluminescence reader, such as a spectrofluorometer. The fluorescence signal is excited by a light source that emits in the UV region of the optics spectrum and within the excitation band of the light harvesting molecule. The emitted fluorescence signal is detected by a photodiode and the wavelength of the signal detected may be limited using a long pass filter which blocks stray emitted light and accepts light with wavelengths at and around the peak emission wavelength of the fluorescence emitting label. In other embodiments, the long pass filter may be replaced by a band pass filter. Furthermore, the excitation light may be limited by a band pass filter. In some embodiments, the luminescence detector is a photoluminescence spectrometer. In some embodiments, the luminescence detector is in the form of a smartphone-based luminescence reader. In some embodiments, the smartphone-based reader has charge-coupled device (CCD) type cameras (CCDC) that detects photoluminescence emission in the visible range.

In some embodiments, the UV light with a wavelength of less than 400 nm is emitted by a light source. In some embodiments, the UV light exposed to the test mixture has a wavelength of 200-400 nm, 250-350 nm, or 280-330 nm. In certain embodiments, the UV light exposed to the test mixture has a wavelength of about 310 nm. In certain embodiments, the UV light exposed to the test mixture has a wavelength of about 320 nm.

In some embodiments, the light source is a light emitting diode. In some embodiments, the light emitting diode is a UV laser diode. The diode may be a UV, LED, or photodiode. In some embodiments, the light emitting diode emits light having a wavelength in a range from about 210 nm to about 290 nm, from about 210 nm to about 290 nm, from about 230 nm to about 270 nm, or about 254 nm. In some embodiments, the light emitting diode emits light having a wavelength in a range from about 290 nm to about 400 nm, from about 330 nm to about 380 nm, or about 365 nm. In some embodiments, the light emitting diode is a hand-held diode.

The test sample may be a sample material known to contain or suspected to contain a rare earth element and/or an actinide element.

In some embodiments, the test sample contains or is suspected to contain rare earth elements including lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc), yttrium (Y), or any combinations thereof. The rare earth elements can be in any oxidation state (e.g., Eu²⁺, Sm³⁺, Ln²⁺, Ln³⁺, Ln⁴⁺, etc.).

In some embodiments, the test sample contains or is suspected to contain at least one rare earth element selected from the group consisting of Sm³⁺, Eu³⁺, Tb³⁺, Dy³⁺, Pr³⁺, and Ho³⁺.

In some embodiments, the test sample contains or is suspected to contain actinide elements including, but not limited to, uranium (U), thorium (Th), plutonium (Pu), americium (Am), actinium (Ac), protactinium (Pa), neptunium (Np), and curium (Cm). The actinide elements can be in any oxidation state (e.g., Cm³⁺, Pu⁴⁺, Bk⁴⁺, NpO²⁺, UO₂²⁺, etc.).

In some embodiments, the test sample contains or is suspected to contain an actinide element which is Cm and/or Am.

In some embodiments, the test sample comprises an additional element that is not a REE or an actinide element. The additional element may be selected from Mg²⁺, Al³⁺, Ca²⁺, Co²⁺, Ni²⁺, Cu²⁺, Fe²⁺, Fe³⁺, Zn²⁺, U, Th, and other alkali, alkaline earth, and transition metals.

The method may include contacting an aqueous solution or a solution comprising water and/or an organic solvent with the test sample to form a test mixture. In an embodiment, the aqueous solution is any water-based solution including, but not limited to, fresh water (e.g., tap water, distilled water, bidistilled water, deionized water, deionized distilled water, reverse osmosis water, well water, wastewater, or fresh water obtained from natural sources such as lakes, groundwaters, streams, rivers, etc.) or salt water such as seawater. In some embodiments, the organic solvent is at least one selected from the group consisting of an alcohol, acetonitrile, dimethylformamide (DMF), and dimethyl sulfoxide (DMSO). Exemplary alcohols include, but are not limited to, monoalcohols (e.g., methanol, ethanol, propanol, isopropanol, n-butanol, isobutanol), polyalcohols including glycols. In some embodiments, the alcohol is ethanol. In some embodiments, the test mixture comprises water and/or alcohol.

In some embodiments, the test sample is pre-processed prior to preparing the test mixture. Non-limiting examples of suitable pre-processing includes acid leaching, bioleaching, ion-exchange extraction, pH adjustment, iron oxide precipitation, temperature cooling (e.g., geothermal brines).

The test mixture may comprise the light-harvesting molecule disclosed herein in any of its embodiments and a buffer. In some embodiments, the light-harvesting molecule is present in an amount of about 0.01 μM-100 mM, about 0.1 μM-50 mM, about 0.5 μM-10 mM, about 1 μM-1 mM, about 5 μM-800 μM, about 10 μM-600 μM, about 20 μM-500 μM, about 30 μM-400 μM, about 40 μM-300 μM, about 50 M-200 μM, or about 75 μM-100 μM, relative to a total volume of the test mixture. In some embodiments, the light-harvesting molecule is present in the test mixture at a concentration of 1-50 μM, 5-40 μM, or 10-20 μM.

In some embodiments, the test mixture has a pH between about 5 to about 11.5, between about 6 to about 11, between about 7 to about 10, between about 8 to about 9. In some embodiments, the test mixture has a mild acidic pH of 5, 5.5, 6, or 6.5. In some embodiments, the test mixture has a neutral pH of about 7. In some embodiments, the test mixture has a basic pH of 7.5, 8, 8.5, 9, 9.5, 10, 10.5, 11, or 11.5.

In some embodiments, the rare earth element is Sm, and a fluorescence emission at 630-670 nm, 640-660 nm, or about 650 nm, is detected upon excitation at a wavelength of 200-400 nm, 250-350 nm, 280-330 nm, 300-320 nm, or 310-320 nm. In some embodiments, the change in fluorescence is detected by naked eye following exposure of the test mixture to an excitation light source.

In some embodiments, the limit of detection for Sm is 100 ppb, 75 ppb, 50 ppb, 25 ppb, 10 ppb, or 1 ppb.

In some embodiments, the rare earth element is Eu, and a fluorescence emission at 600-630 nm, 610-620 nm, or about 615 nm, is detected upon excitation at a wavelength of 200-400 nm, 250-350 nm, 280-330 nm, 300-320 nm, or 310-320 nm. In some embodiments, the change in fluorescence is detected by naked eye following exposure of the test mixture to an excitation light source.

In some embodiments, the limit of detection for Eu is 100 ppb, 75 ppb, 50 ppb, 25 ppb, 10 ppb, or 1 ppb.

In some embodiments, the rare earth element is Tb, and a fluorescence emission at 520-560 nm, 530-550 nm, or about 540 nm, is detected upon excitation at a wavelength of 200-400 nm, 300-350 nm, or 310-330 nm. In some embodiments, the change in fluorescence is detected by naked eye following exposure of the test mixture to an excitation light source.

In some embodiments, the limit of detection for Tb is 100 ppb, 75 ppb, 50 ppb, 25 ppb, 10 ppb, 1 ppb, 0.1 ppb, or 0.01 ppb.

In some embodiments, the actinide element is Cm, and a fluorescence emission at 600-630 nm, 610-620 nm, or about 615 nm, is detected upon excitation at a wavelength of 200-400 nm, 250-350 nm, 280-330 nm, 300-320 nm, or 310-320 nm. In some embodiments, the change in fluorescence is detected by naked eye following exposure of the test mixture to an excitation light source.

In some embodiments, the limit of detection for Cm is 100 ppb, 75 ppb, 50 ppb, 25 ppb, 10 ppb, 1 ppb, 0.1 ppb, or 0.01 ppb.

In some embodiments, the rare earth element is Dy, and a fluorescence emission at 550-590 nm, 560-580 nm, or about 570 nm, is detected upon excitation at a wavelength of 200-400 nm, 250-350 nm, or 280-330 nm. In some embodiments, the change in fluorescence is detected by naked eye following exposure of the test mixture to an excitation light source.

In some embodiments, the limit of detection for Dy is 100 ppb, 75 ppb, 50 ppb, 25 ppb, 10 ppb, or 1 ppb.

Another aspect of the present disclosure relates to a method for detecting the presence of a rare earth element and/or an actinide element in a test sample, the method comprising: combining the test sample with the light-harvesting molecule disclosed herein in any of its embodiments, which is capable of producing luminescence upon interaction with a rare earth element or an actinide element to form a solid test mixture; exposing the solid test mixture to UV light with a wavelength of less than 400 nm; and determining that the rare earth element and/or the actinide element is present in the test sample when a luminescent signal is detected by a luminescence detector.

The test sample may be in a solid state (e.g., powder, particle, crystalline state). The test sample may be a sample material known to contain or suspected to contain a rare earth element and/or an actinide element.

In some embodiments, the test sample contains or is suspected to contain rare earth elements including lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc), yttrium (Y), or any combinations thereof. The rare earth elements can be in any oxidation state (e.g., Eu²⁺, Sm³⁺, Ln²⁺, Ln³⁺, Ln⁴⁺, etc.).

In some embodiments, the test sample contains or is suspected to contain at least one rare earth element selected from the group consisting of Sm, Eu, Tb, Dy, Pr, and Ho.

In some embodiments, the test sample contains or is suspected to contain actinide elements including, but not limited to, uranium (U), thorium (Th), plutonium (Pu), americium (Am), actinium (Ac), protactinium (Pa), neptunium (Np), and curium (Cm). The actinide elements can be in any oxidation state (e.g., Cm³⁺, Pu⁴⁺, Bk⁴⁺, NpO²⁺, UO₂²⁺, etc.).

In some embodiments, the test sample contains or is suspected to contain an actinide element which is Cm and/or Am.

In some embodiments, the rare earth element is Sm, and a fluorescence emission at 630-670 nm, 640-660 nm, or about 650 nm, is detected upon excitation at a wavelength of 200-400 nm, 250-350 nm, or 280-330 nm. In some embodiments, the change in fluorescence is detected by naked eye following exposure of the test mixture to an excitation light source.

In some embodiments, the rare earth element is Eu, and a fluorescence emission at 600-630 nm, 610-620 nm, or about 615 nm, is detected upon excitation at a wavelength of 200-400 nm, 250-350 nm, or 280-330 nm. In some embodiments, the change in fluorescence is detected by naked eye following exposure of the test mixture to an excitation light source.

In some embodiments, the rare earth element is Tb, and a fluorescence emission at 520-560 nm, 530-550 nm, or about 540 nm, is detected upon excitation at a wavelength of 200-400 nm, 250-350 nm, or 280-330 nm. In some embodiments, the change in fluorescence is detected by naked eye following exposure of the test mixture to an excitation light source.

In some embodiments, the rare earth element is Dy, and a fluorescence emission at 550-590 nm, 560-580 nm, or about 570 nm, is detected upon excitation at a wavelength of 200-400 nm, 250-350 nm, or 280-330 nm. In some embodiments, the change in fluorescence is detected by naked eye following exposure of the test mixture to an excitation light source.

In some embodiments, the solid test mixture is substantially free of a solvent. In some embodiments, the solid test mixture is free of a solvent. As used herein, the phrase “substantially free” refers to any solvent that is present in an amount of less than about 0.0001%, less than about 0.001%, less than about 0.01%, less than about 0.1%, less than about 1%, or less than about 5%, relative to a total weight or volume of the solid test mixture.

In some embodiments, the test sample comprises an additional element that is not a REE or an actinide element. The additional element may be selected from Li⁺, Na⁺, K⁺, Rb⁺, Cs⁺, Mg²⁺, Ra²⁺, Al³⁺, Ca²⁺, Co²⁺, Ni²⁺, Cu²⁺, Fe²⁺, Fe³⁺, Zn²⁺, UO₂²⁺, Th⁴⁺, and other alkali, alkaline earth, and transition metals.

### EXAMPLES

### Example 1: Development of a Practical and Low-Cost Method for Detection and Analysis of Rare Earth Elements and Actinides

REEs are key ingredients of strategic industrial and national security-related sectors, and can be used in many applications, including in catalysis, hard drives, electronics, magnets, optics, MRI exams, lighting, electric engines, electric car batteries, polishing, laser amplification. REEs are also present in nuclear waste, along with actinide elements. However, severe and sustained tensions on the REE supply chain has put pressure on research and industrial activities. Although there are many mining projects and research initiatives on REEs to help reduce the pressure, there is a bottleneck in the ability to analyze the mining samples in that there is that there is currently no simple way to analyze such samples to determine whether REEs are present. Current technologies-Inductively Coupled Plasma (ICP) analysis, UV-visible spectrophotometry (Arzenazo assay), and Optical sensors-all have their advantages, but also have limitations that contribute to the bottleneck.

ICP analysis is currently the preferred method and works for detecting all REEs, but it is costly (e.g., >100 USD/sample), requires research-grade instrument & argon supply, the protocols are lengthy, and does not allow for in-field analysis.

Arzenazo assays are inexpensive and uses a commercially available reagent, but can only give a total REE concentration, is non-selective, has a lot of interferences, and the reagent contains arsenic.

Optical sensors are fluorescence-based, easy to implement, and can be adapted for rapid in-field analysis. However, they only work for a single REE and the reagents used are made with in-house molecules that are expensive and not widely available.

Consequently, the goal of this project was to develop a practical and low-cost method for detection and analysis of REEs and actinides. The criteria that were used to develop the assay were (i) must be low cost (<$1/analysis), (ii) must be fast reading, (mist be compatible with in-field analysis, (iii) must work for multiple REEs, (iv) must be able to be used commercially with available components, (v) must have low detection limits, and (vi) ability to extend the assay to detect actinide elements. In the past, attempts to design new molecules to render REE molecules fluorescent in the field did not work and have remained lab-scale because they were too costly and are not available to others. In addition, as discussed above, they typically only work for 1 REE (Tb or Eu). Here, molecules that are less costly and used for different applications were screened to develop an assay that has the following characteristics: first, obtain an original sample that requires little to no preparation, uses a rapid measurement that leads to a quick result. That approach has led to the development of the analytical method and assay described herein.

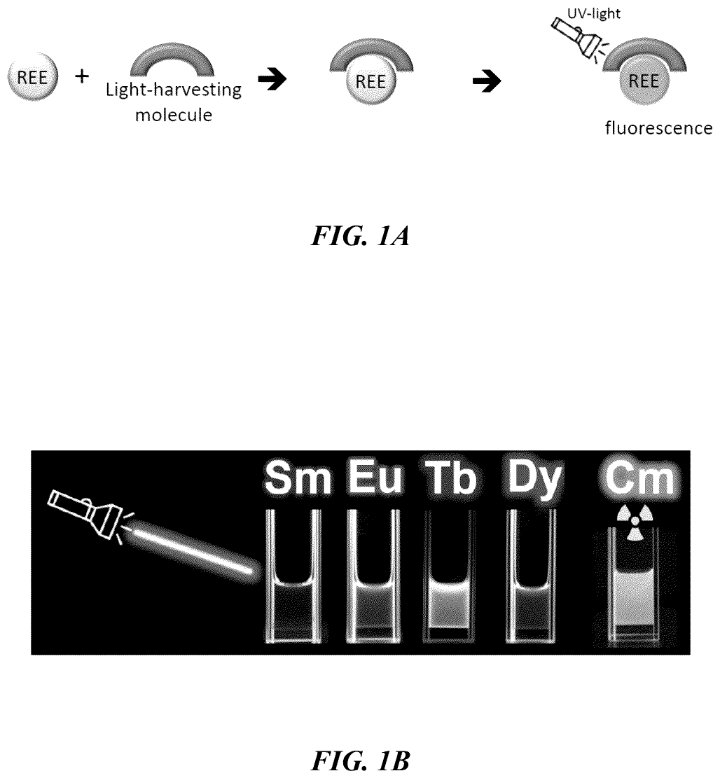

The assay uses the fluorescence properties of REEs to develop a practical fluorescence-based system. First, a sample (e.g., a homogenized sample harvested from the field) that is suspected of containing REEs is added to an aqueous solution containing a light harvesting molecule and adjusted to a pH 6-11. If the sample contains an REE, it binds to the light harvesting molecule, which, when exposed to UV-light, produces a fluorescent signal (FIG. 1A).

In developing the fluorescence-based system discussed herein, it was discovered that some molecules render certain lanthanide and actinide elements highly fluorescent. The molecules, referred to herein as light harvesting molecules. Two such molecules were tested in the examples below and are known as ciclopirox and piroctone. Those molecules are not sold for applications involving the detection of REEs and actinides via fluorescence. They are instead used as additives in cosmetic products and/or in the pharmaceutical industry. The study of their fluorescence properties with REE or actinides revealed that they can be used to develop fluorescence-based detection of REEs and certain actinides (curium and americium). There are other molecules that may be useful for REE chemistry, including pyridoxatin.

Ciclopirox and piroctone are bidentate 1,2-hydroxypyridinone molecules, without an amide bond on the aromatic hydroxypyridinone cycle. Prior efforts in the literature focused on molecules, in particular custom-made molecules of higher denticity (ex: tetradentate, hexadentate, octadentate) or molecules that are not 1,2-hydroxypyridinone or molecules that contain amide bounds (which are likely deleterious to the targeted fluorescence properties).

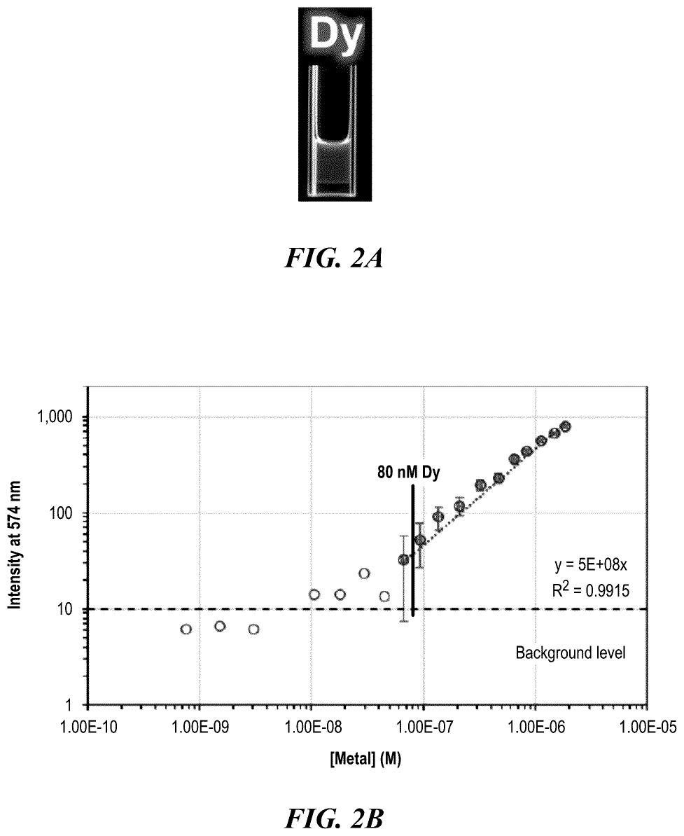

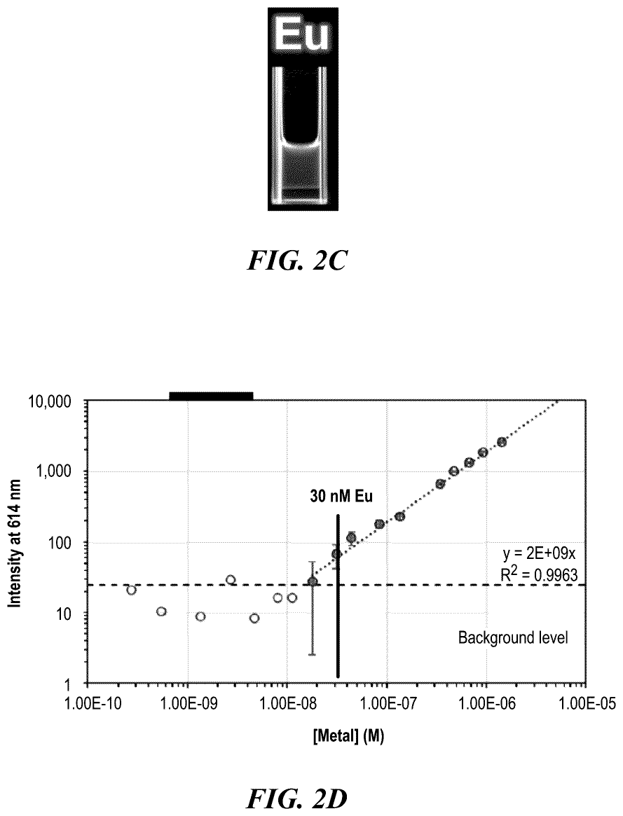

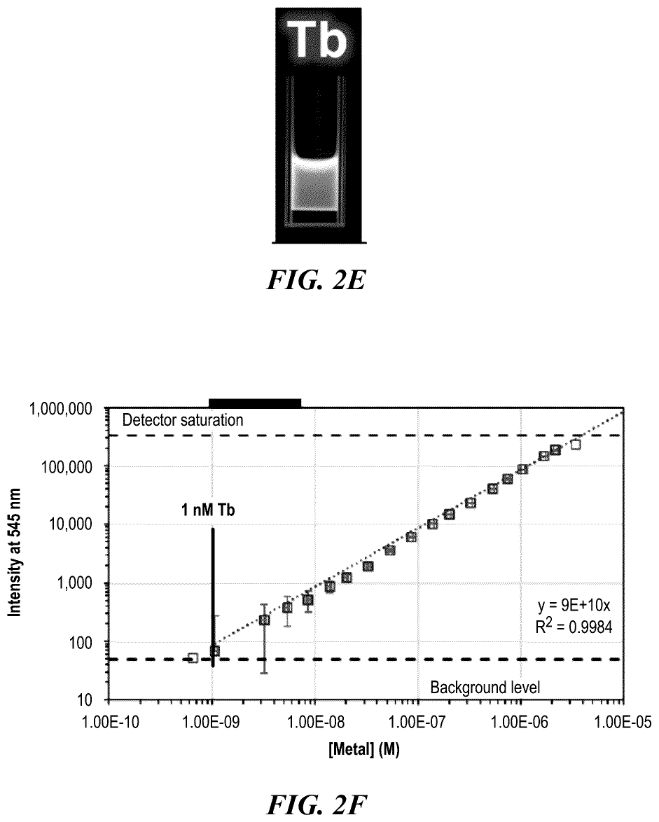

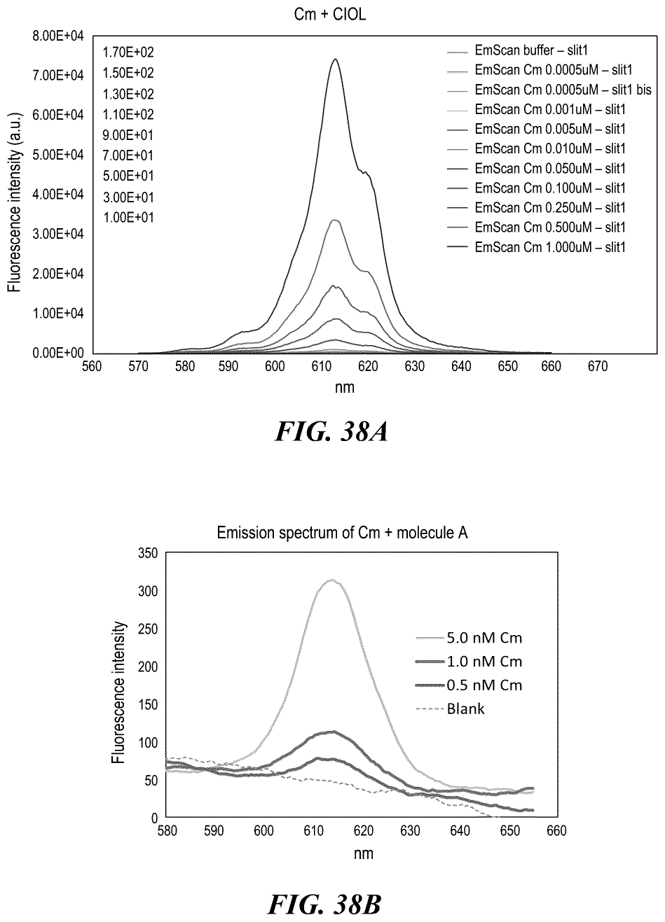

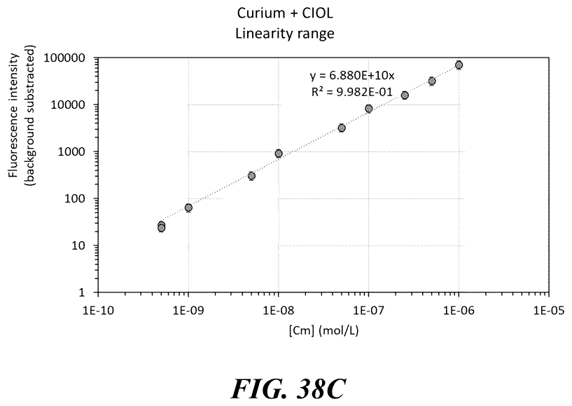

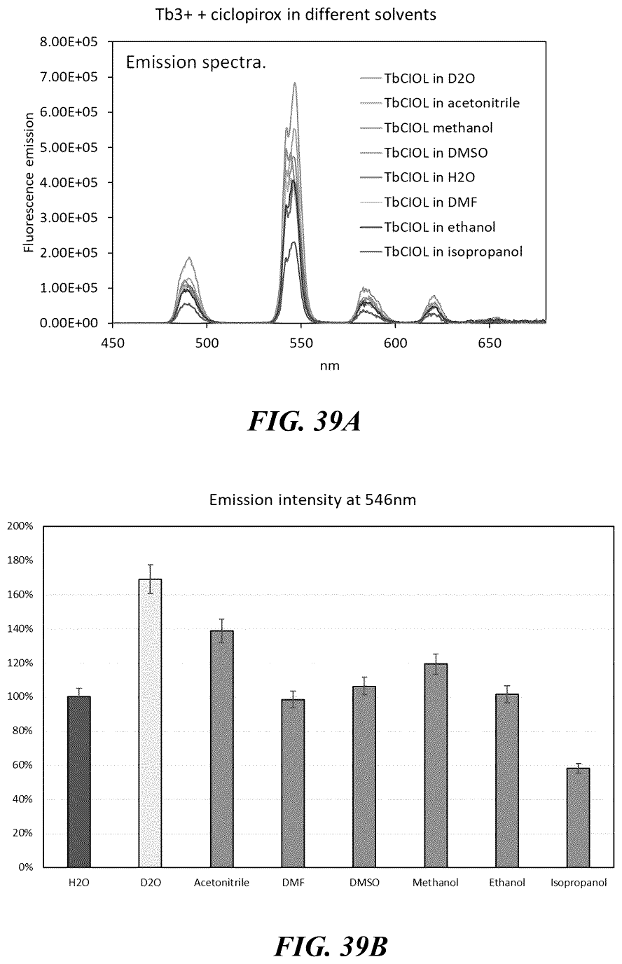

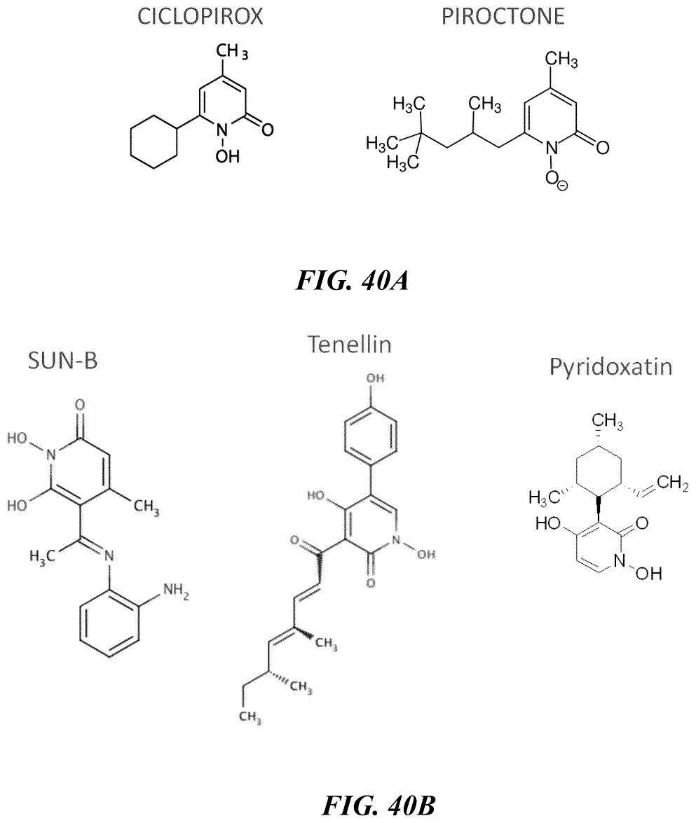

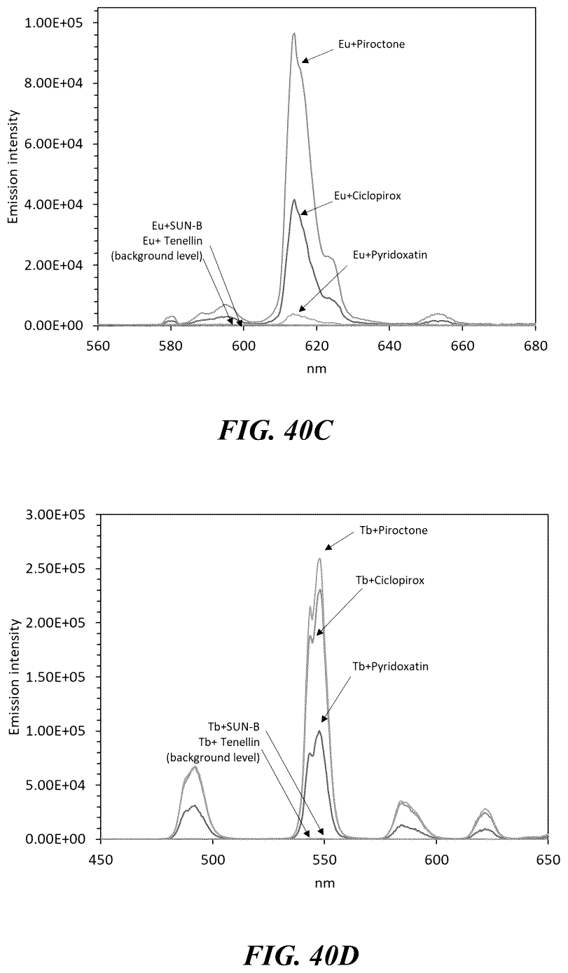

In this example, the light harvesting molecule used was ciclopirox, which is normally sold as a cosmetic product additive at low cost (<50 USD/g in the USA). FIG. 1B shows a group of REE (˜75 μg) and actinide in an aqueous samples pictured under UV light where the mass of the light harvesting molecule is usually ≤10 mg. Preliminary results showing a coarse estimation of detection limits for the Dy, Eu, and Tb samples are shown in FIGS. 2A-2F. These results (see also FIG. 38C for Cm) show that fluorescence detection is a fast technique (<1 second acquisition), extremely affordable (<0.001 USD per sample analysis), and compatible with live monitoring.

As a result of these tests a plan was developed to adapt the system for use in the field. For REEs, select commercial molecules are screened for REE luminescence, then a commercialization-ready formulation for REE detection and analysis (e.g., those compositions discussed herein) is developed. Then the system can be adapted for in-field analysis with, e.g., a fluorescence handheld device and a microfluidic chip for rapid analysis.

The system could then be extended to detect lanthanide fission products, infrared-emitting lanthanides such as Yb³⁺, Nd³⁺, Pr³⁺, and Sm³⁺, and actinide (Cm, Am), and plutonium fluorescence could also be investigated to develop a fluorescence-based sensor for plutonium monitoring.

Summary Fluorescence Data: f-Element Chelators/Organics

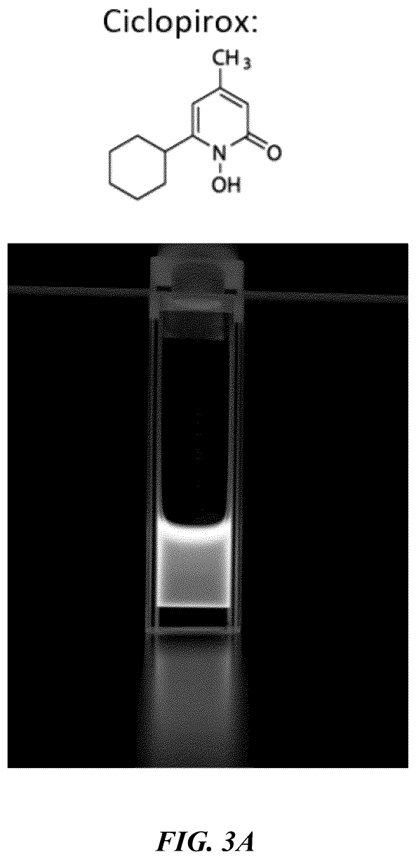

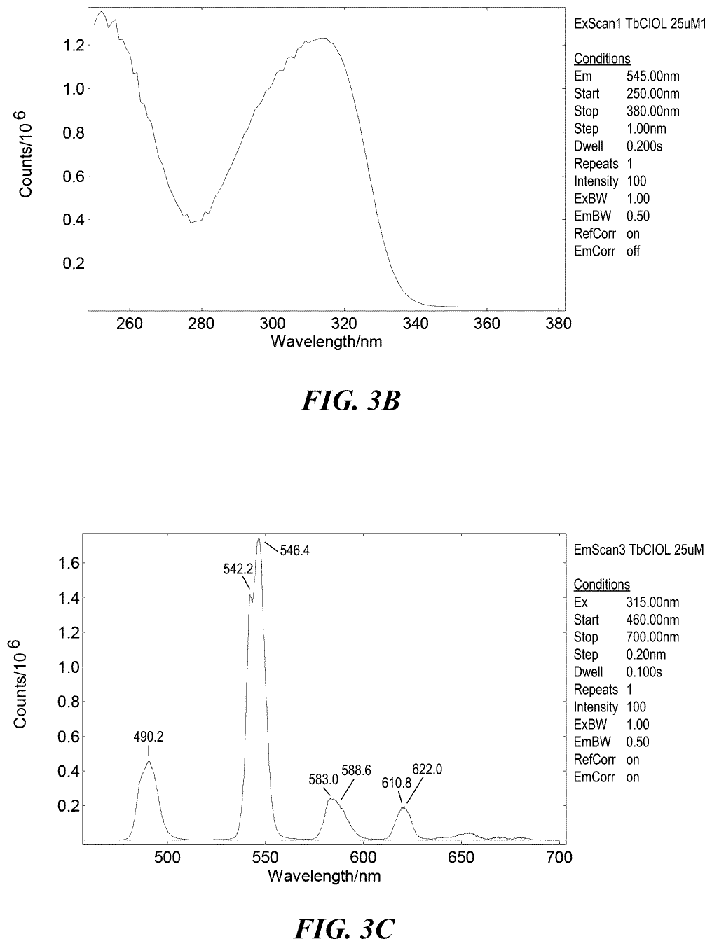

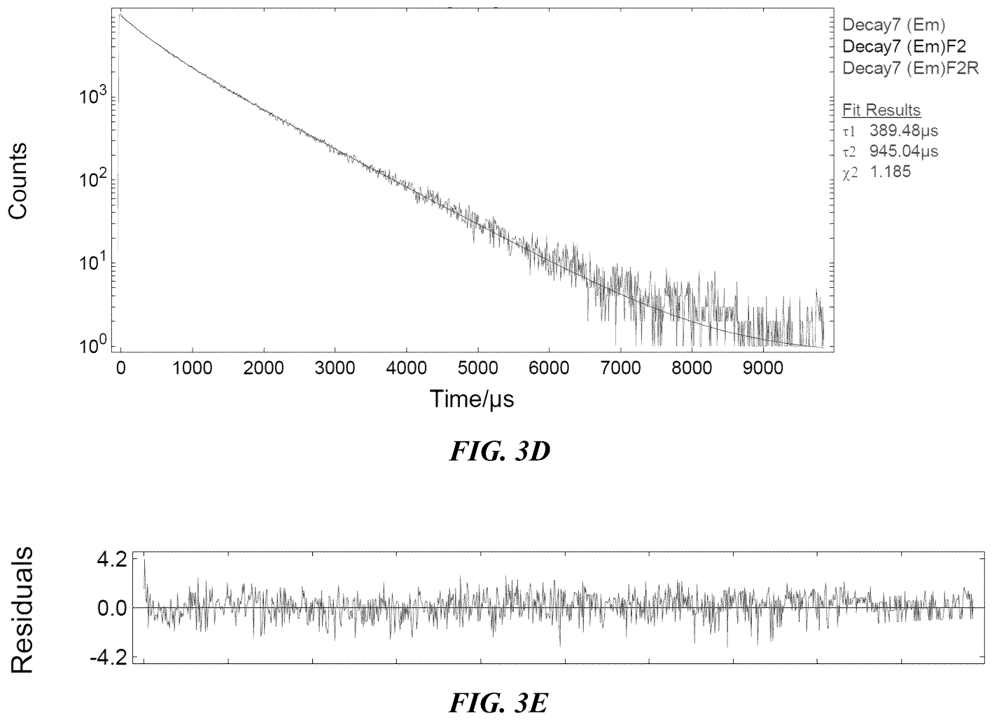

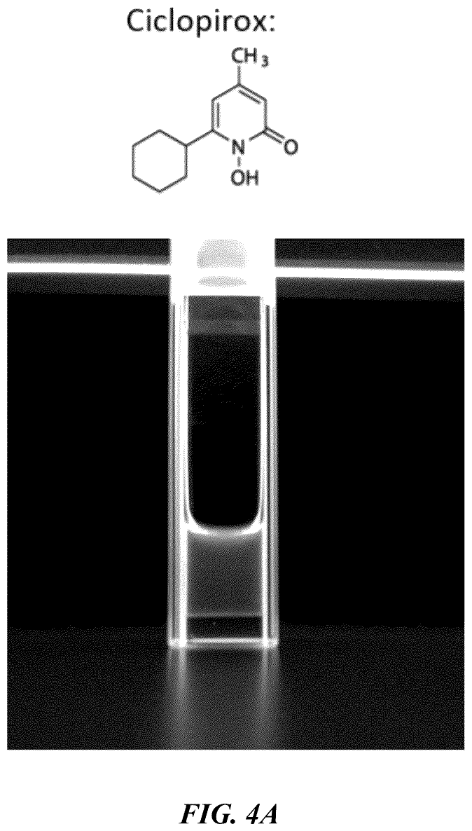

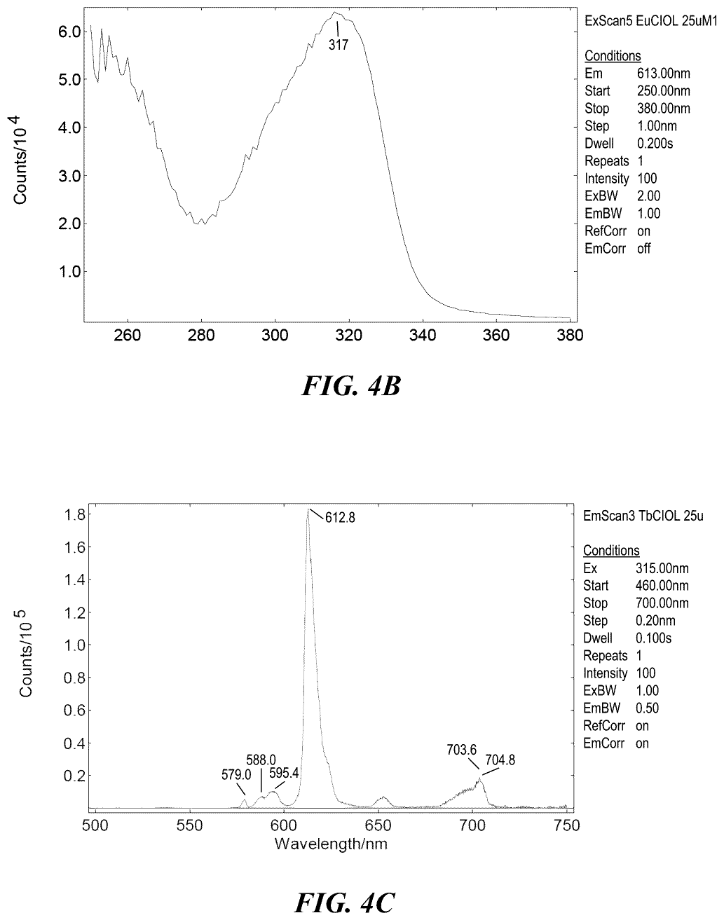

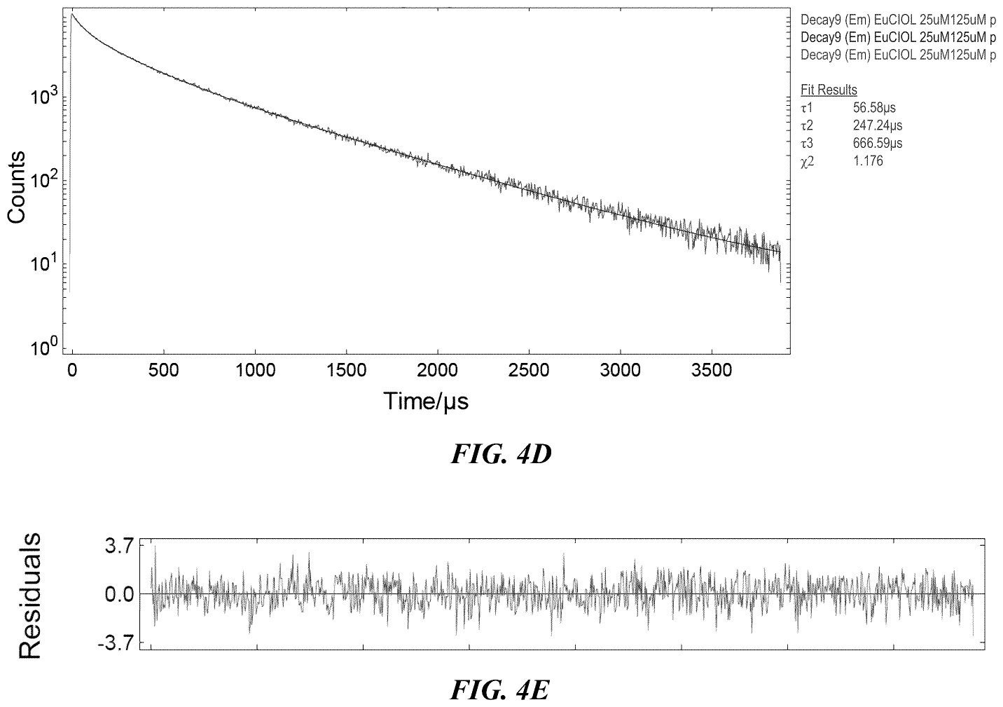

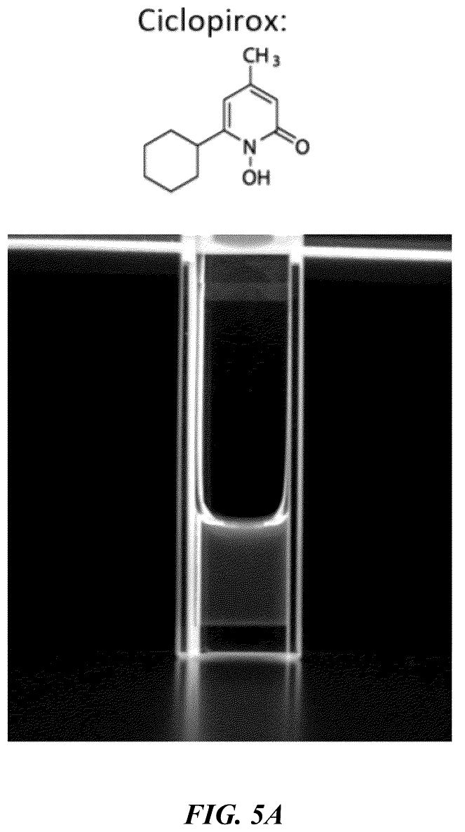

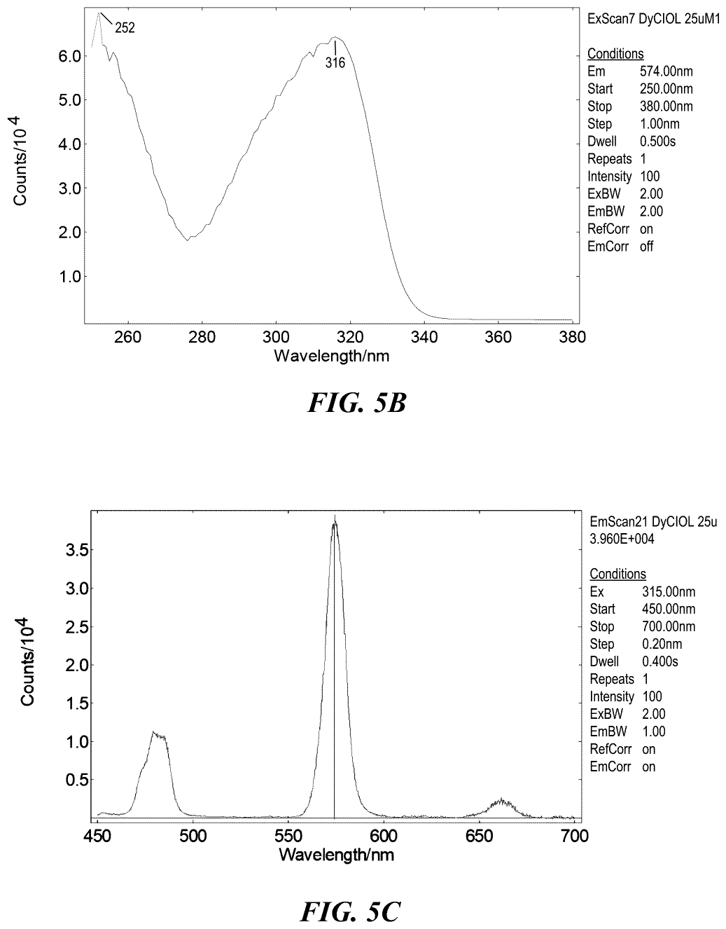

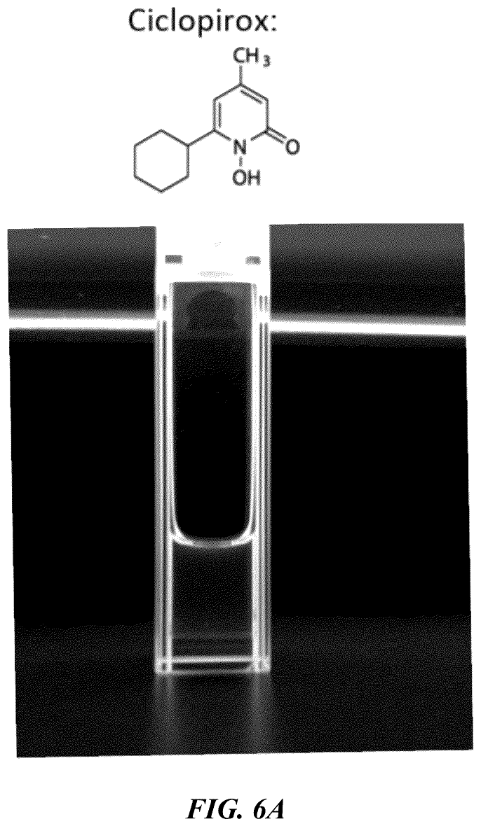

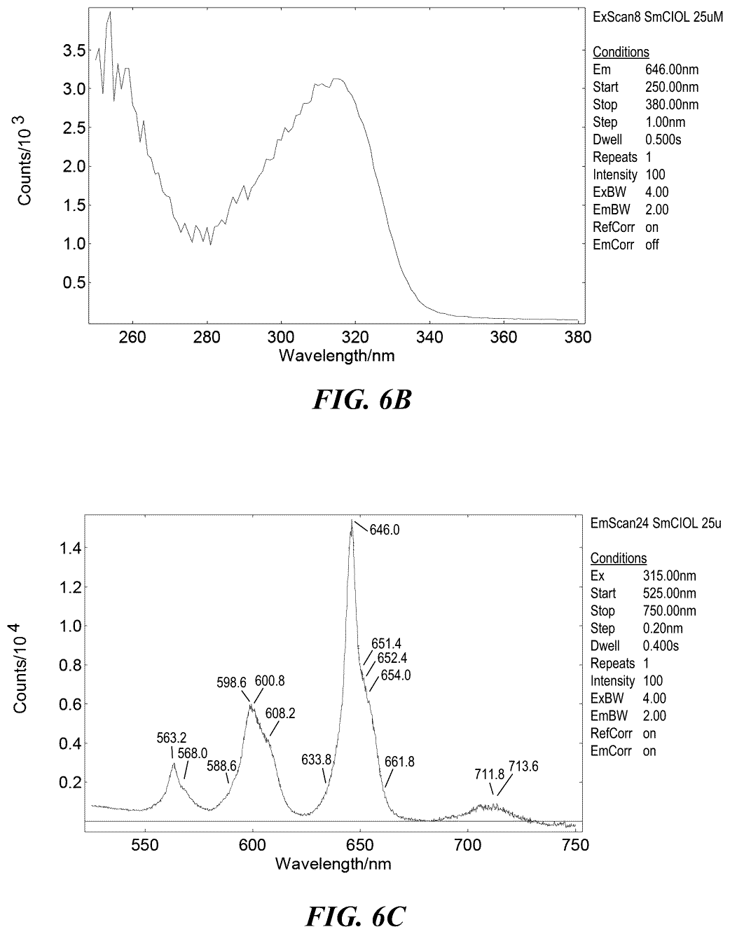

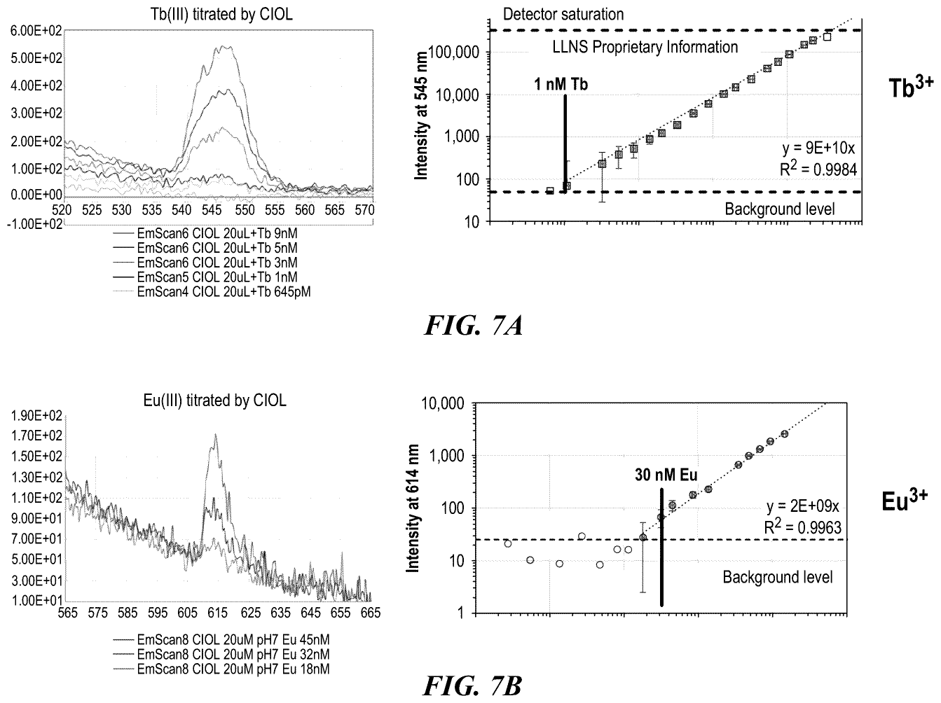

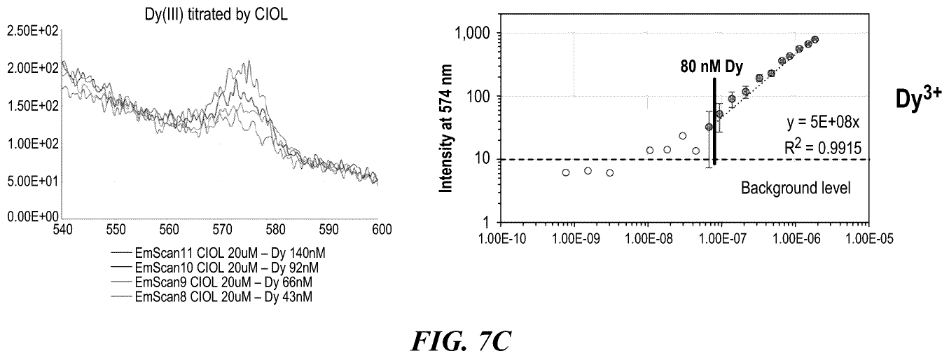

Using an FLS1000 Photoluminescence Spectrometer (Edinburgh Instruments), fluorescence output and decay was measured in samples of Tb(III) with ciclopirox (FIGS. 3A-3E), Eu(III) with ciclopirox (FIGS. 4A-4E), Dy(III) with ciclopirox (FIGS. 5A-5C), and Sm(III) with ciclopirox (FIGS. 6A-6C). Detection limits for Tb³⁺, Eu³⁺, and Dy³⁺ were determined and shown to be detectable starting at a level of about 1-80 nM (see FIGS. 7A-7C), indicating a highly sensitive system that prevents other contaminants in the field sample from causing false positives. That is because the detectable level of the concentration for the REEs is so low, the dilution factor can be very high. Consequently, any other metals in the sample would be present a very low concentration in the analytical sample (due to dilution) far below their detectable limit, and hence not interfere.

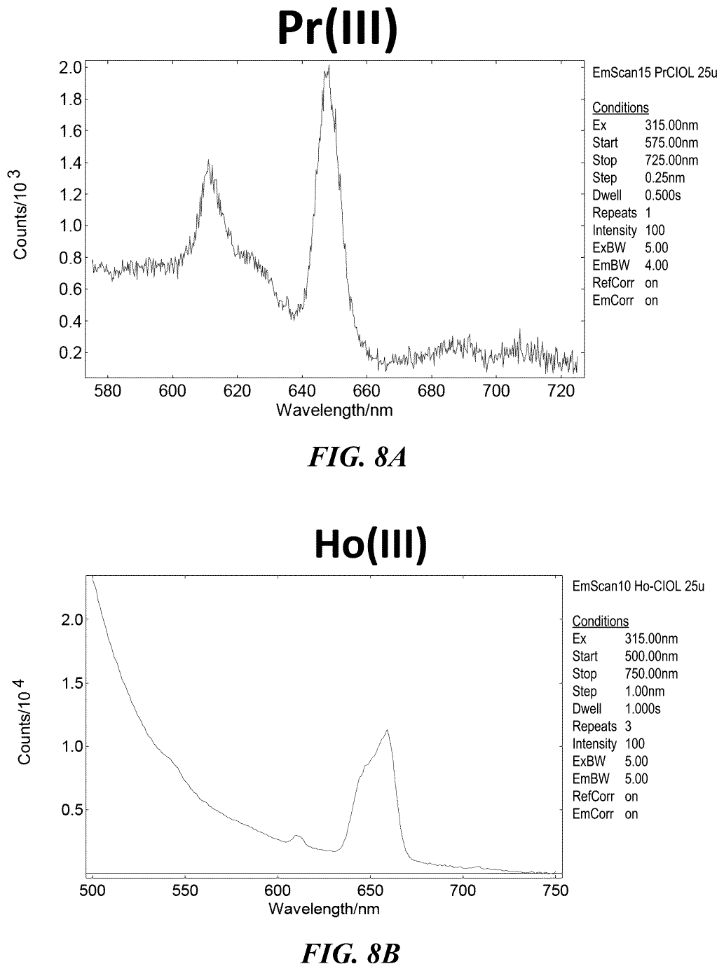

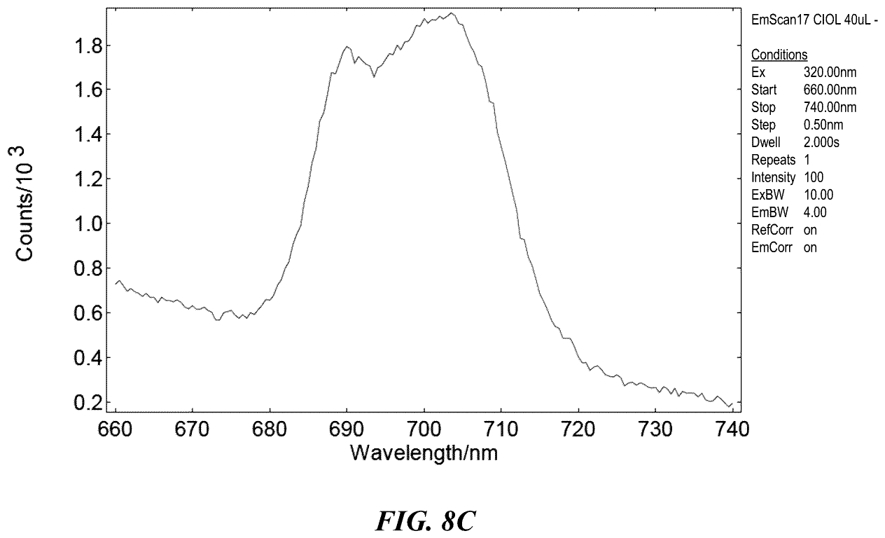

Ciclopirox was also used to test whether other REEs could be detected via fluorescence. As shown in FIGS. 8A-8C, an aqueous solution containing ciclopirox also sensitizes the luminescence of praseodymium (Pr(III)) (FIG. 8A), holmium (Ho(III)) (FIG. 8B), and americium (Am(III)) (FIG. 8C).

### Samples: Overview

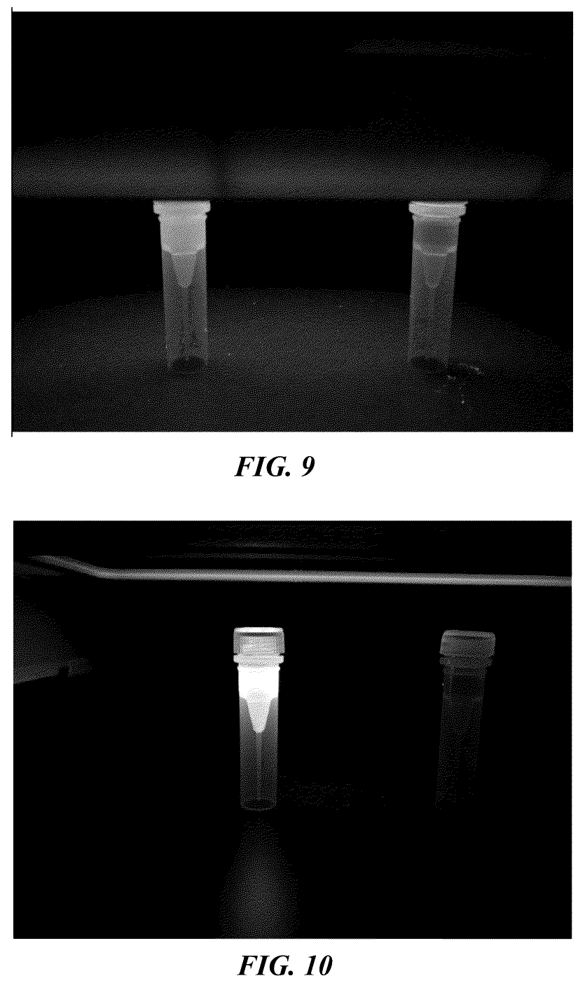

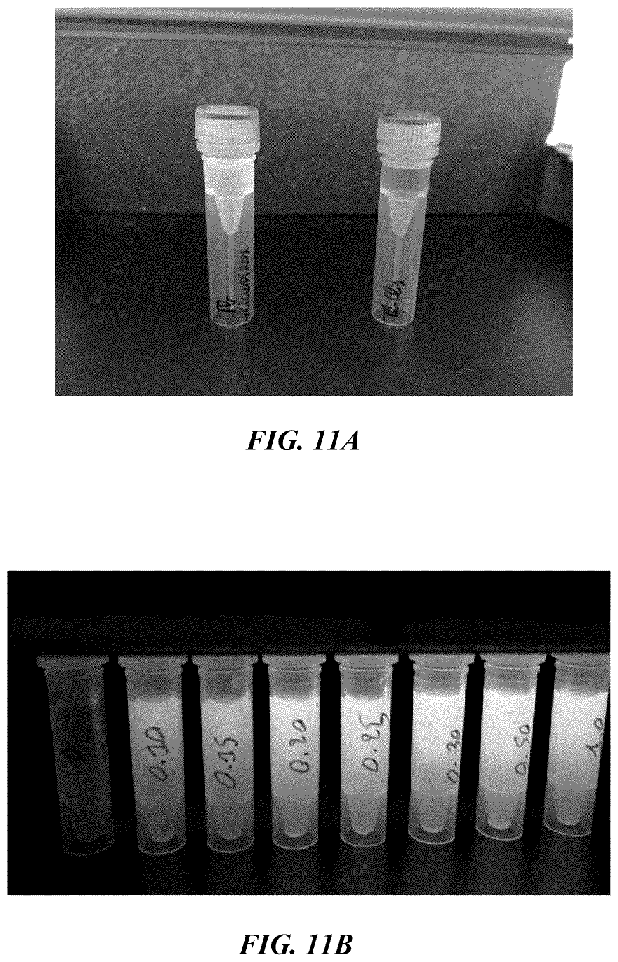

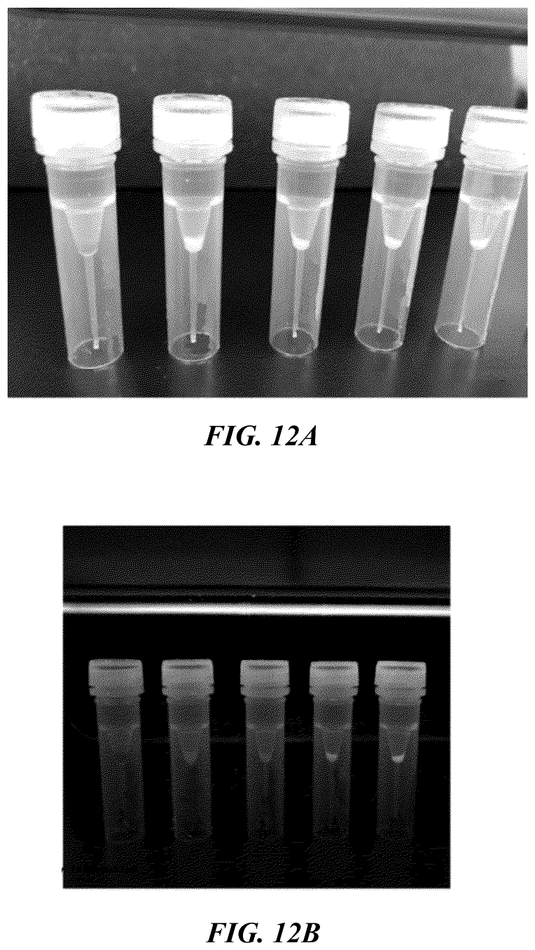

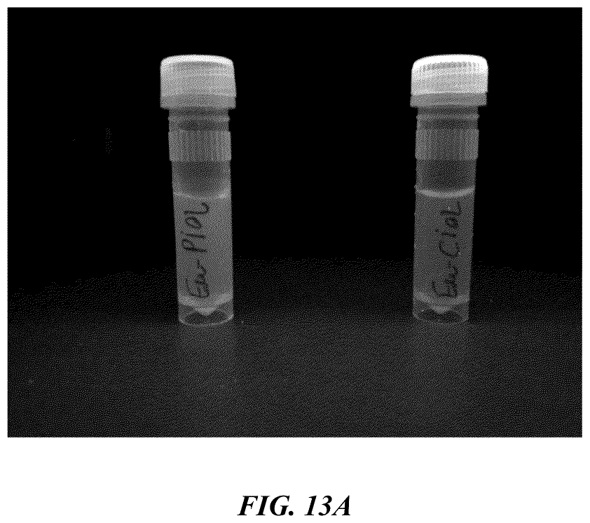

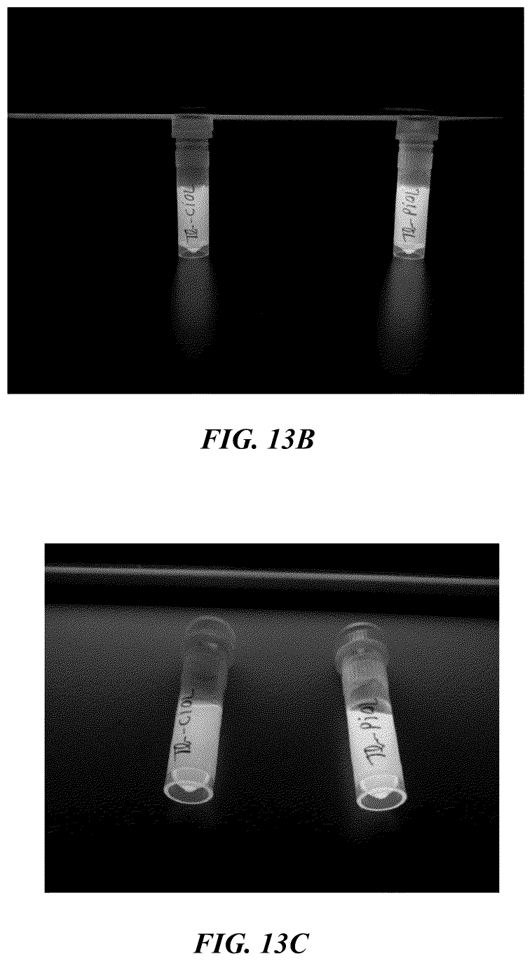

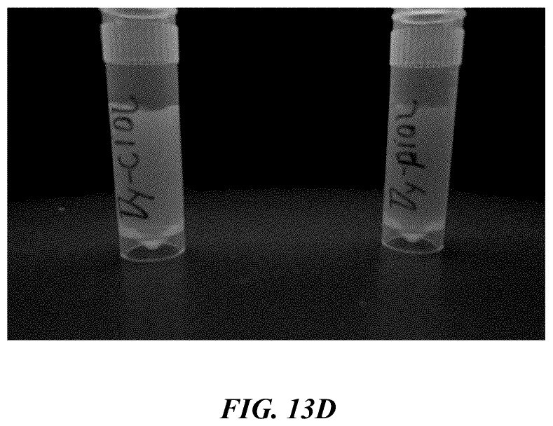

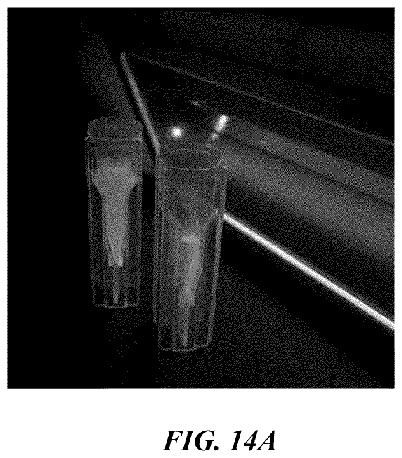

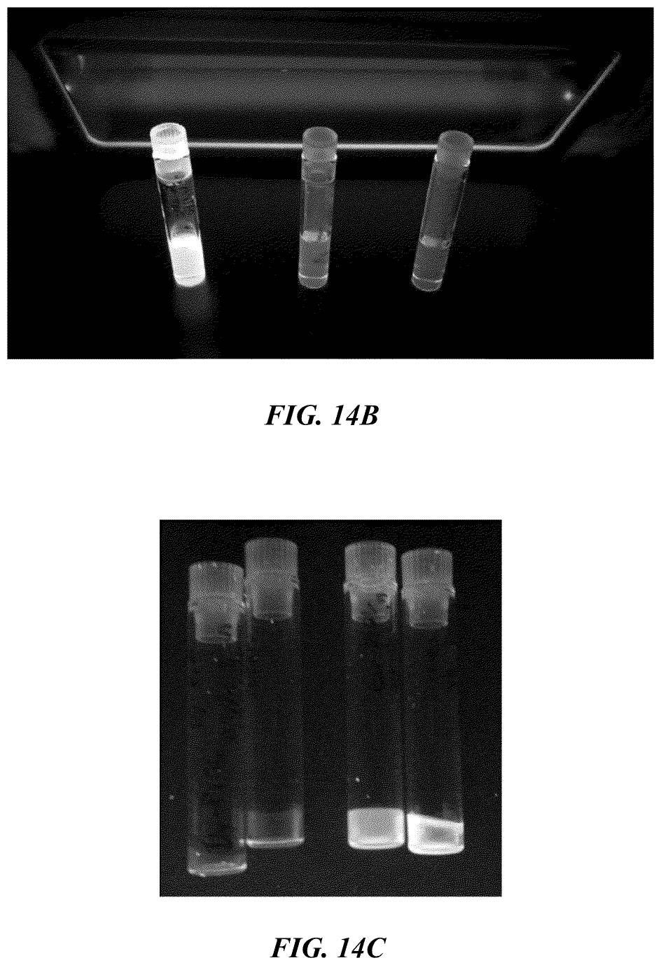

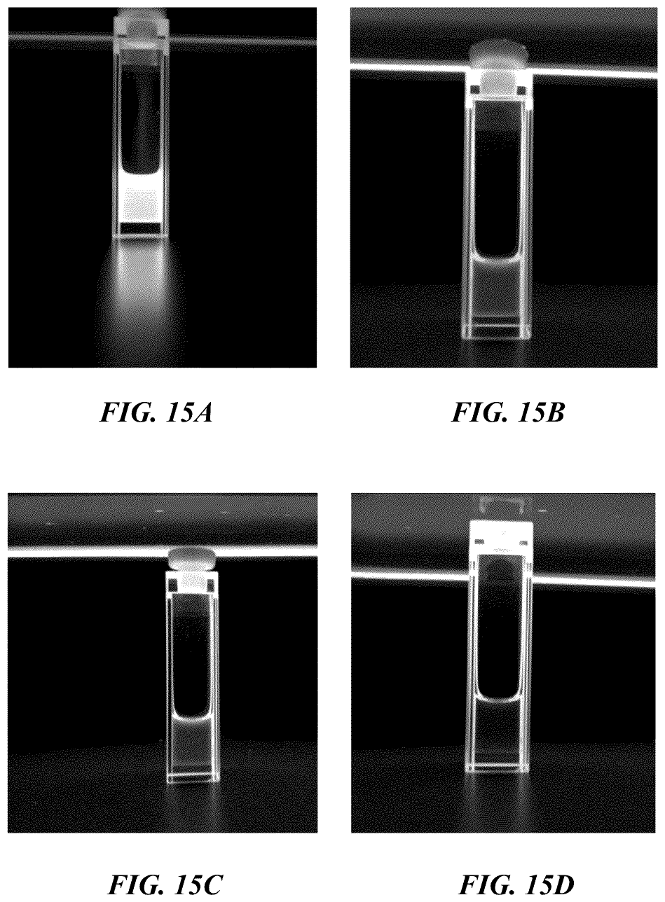

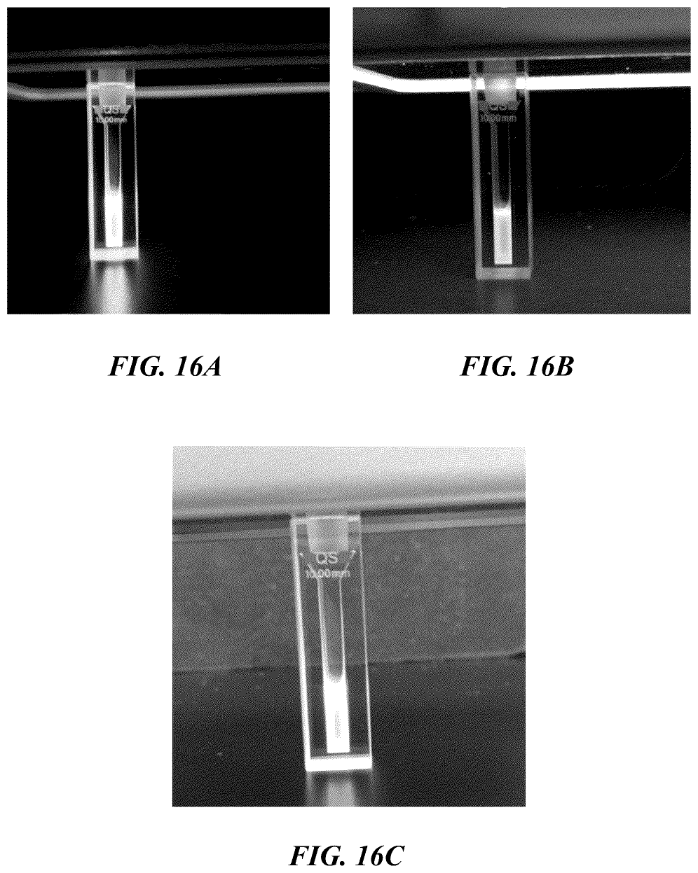

REE Samples. Several samples containing ciclopirox and piroctone and REEs confirmed that those molecules can be used to sensitize the luminescence of REEs and facilitate their detection. Samples containing ciclopirox or piroctone with REEs are shown in FIGS. 9, 10, 11A-11B, 12A-12B, 13A-13D, 14A-14C, 15A-15D, and 16A-16C.

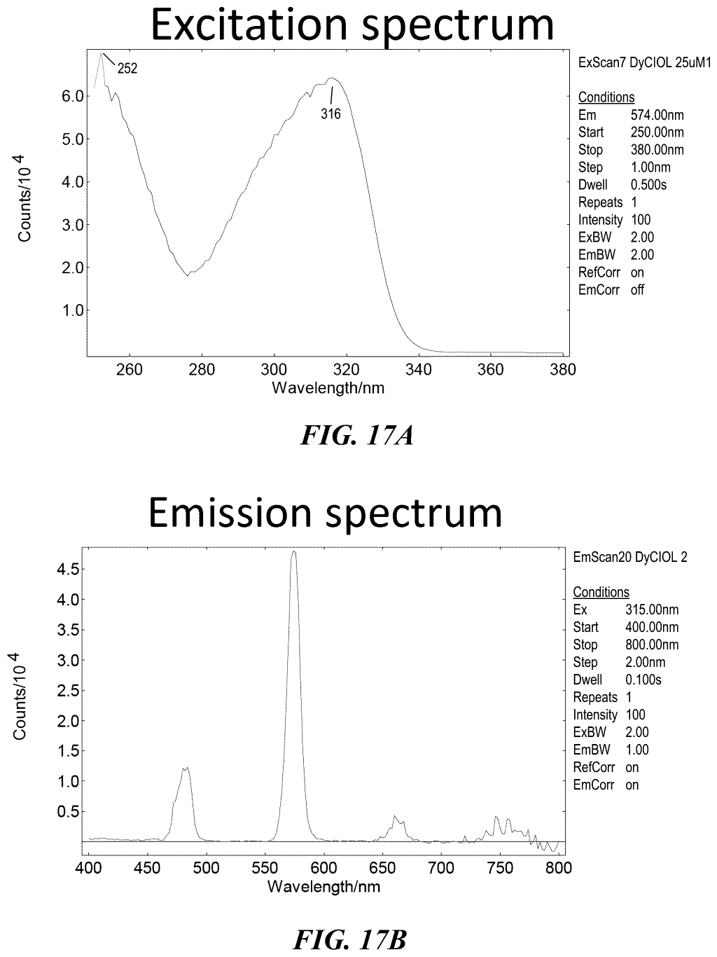

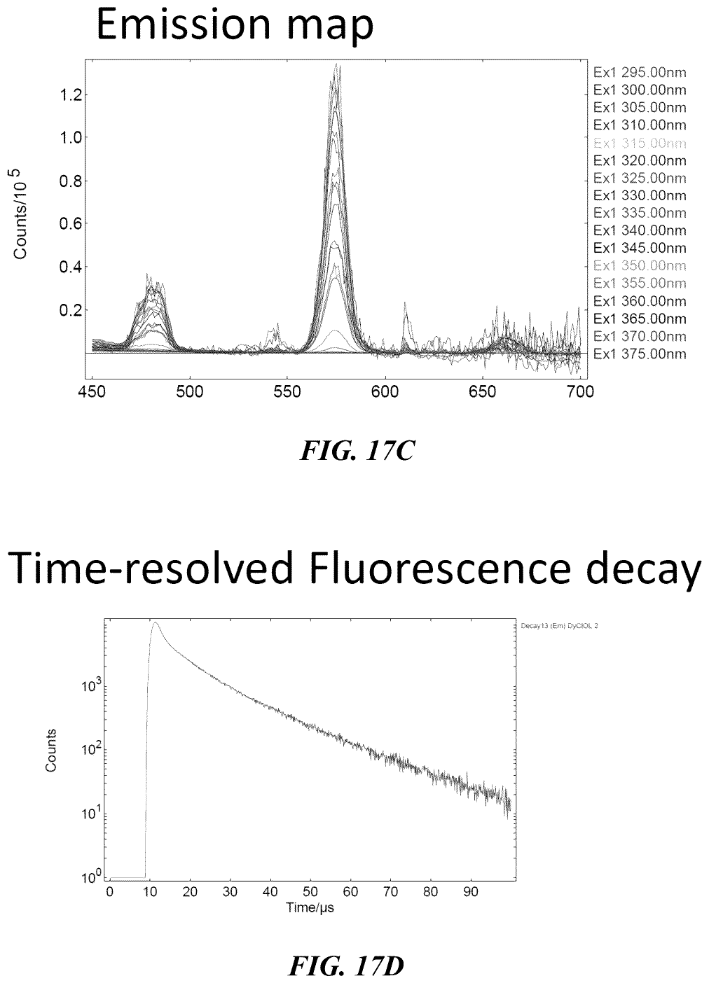

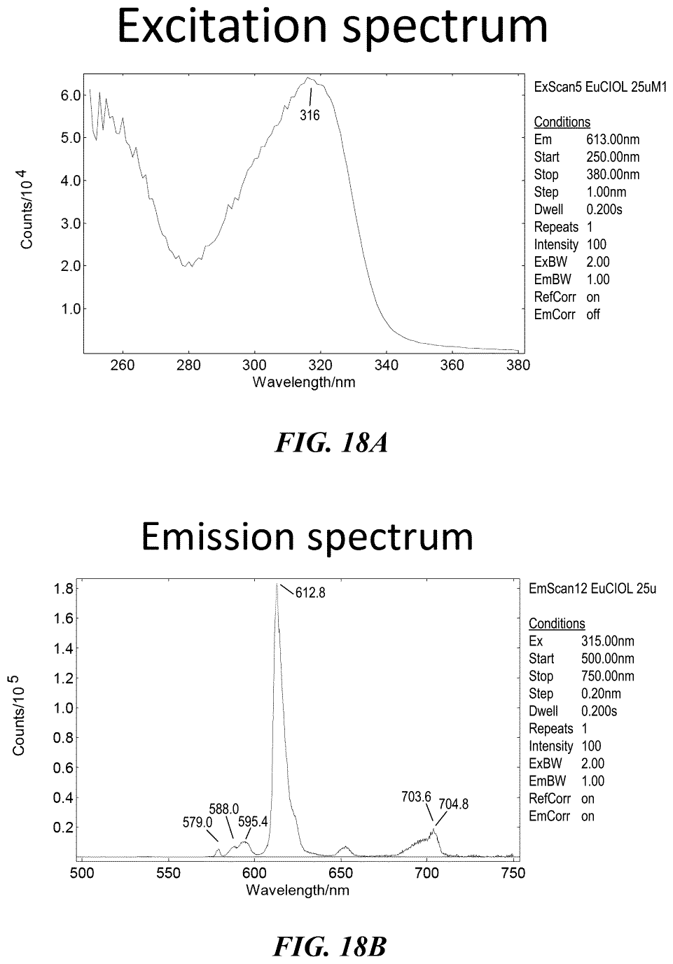

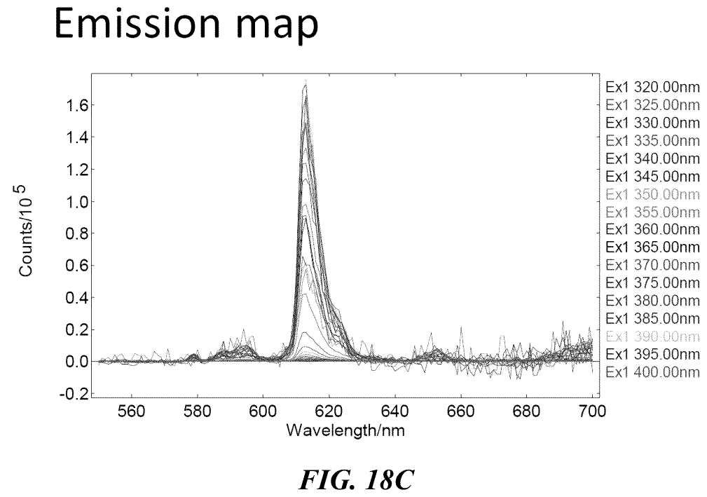

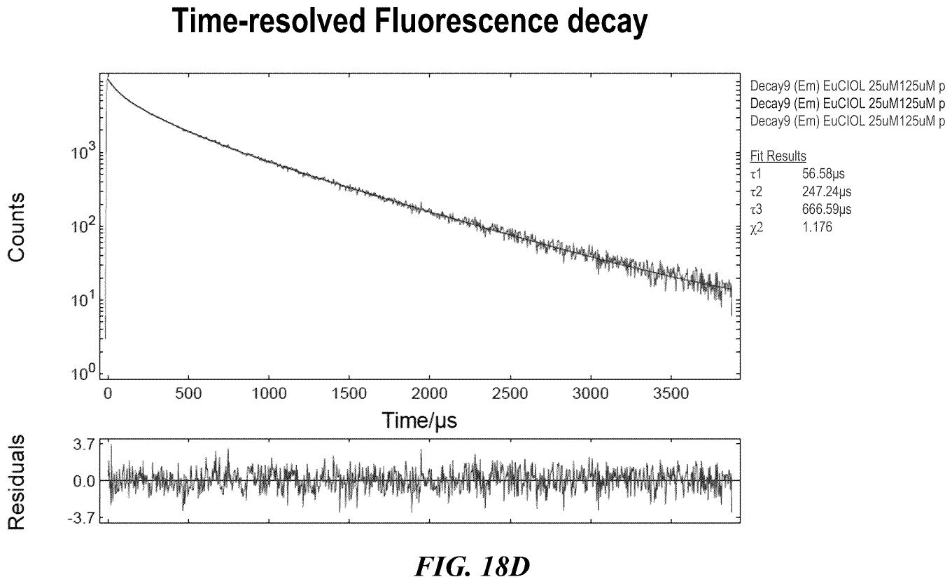

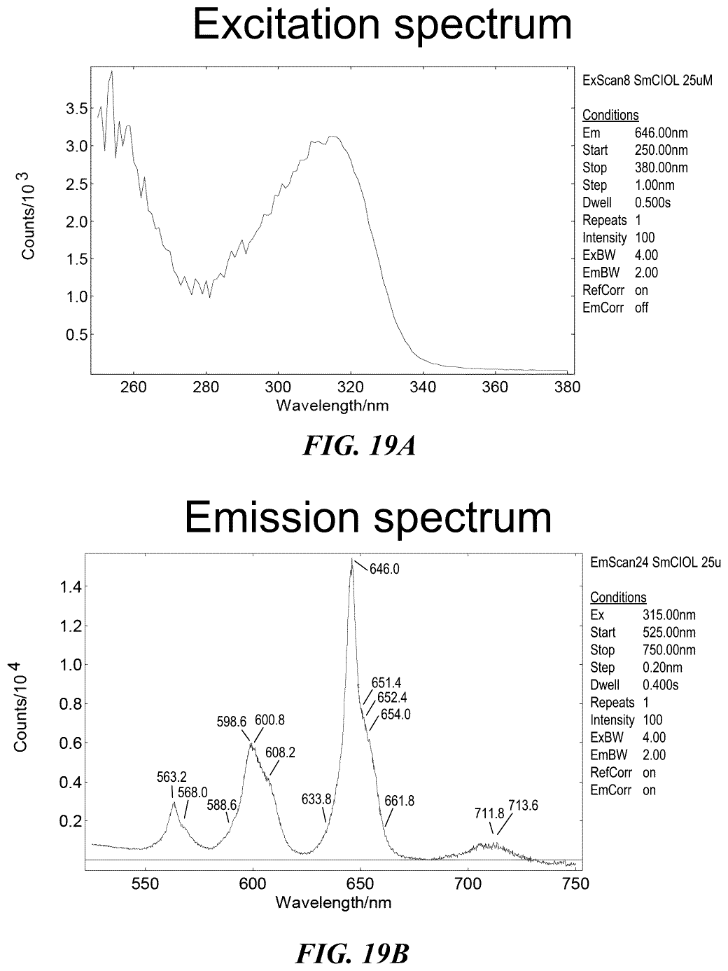

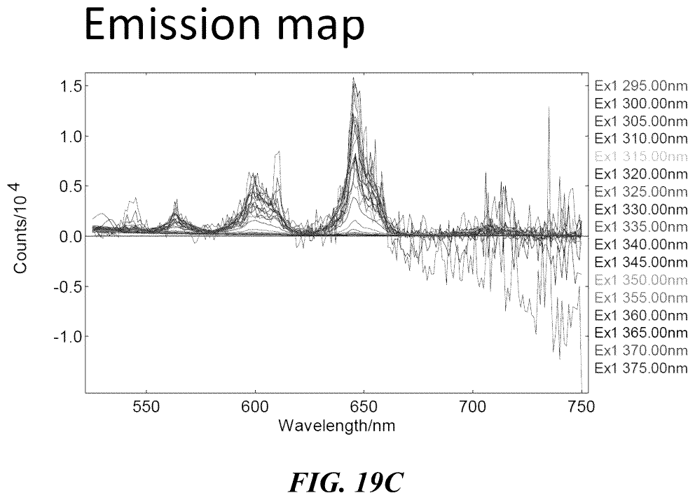

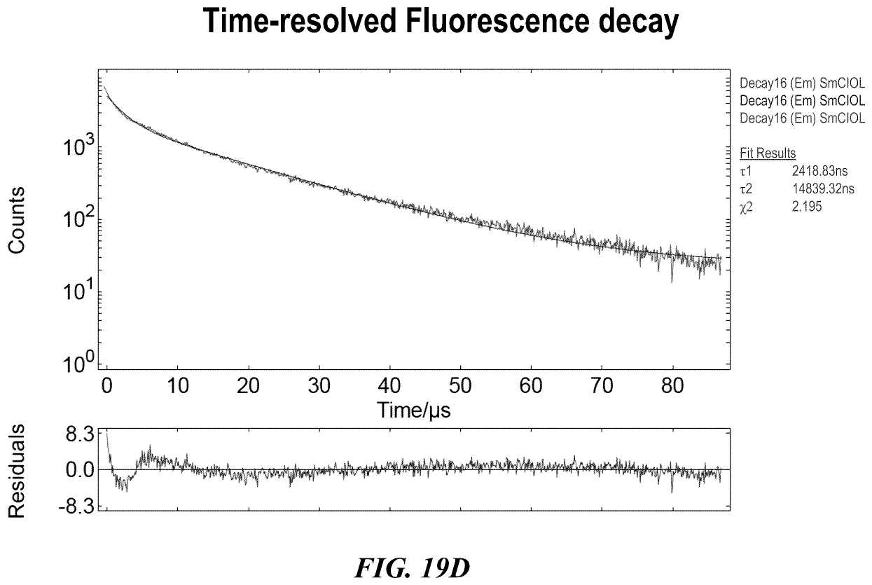

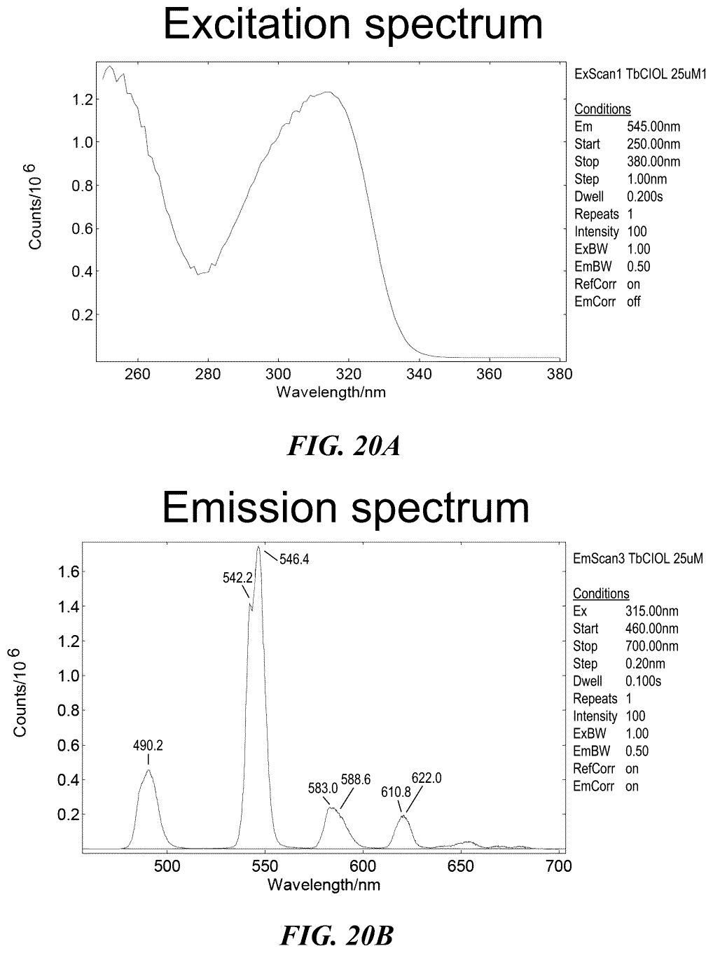

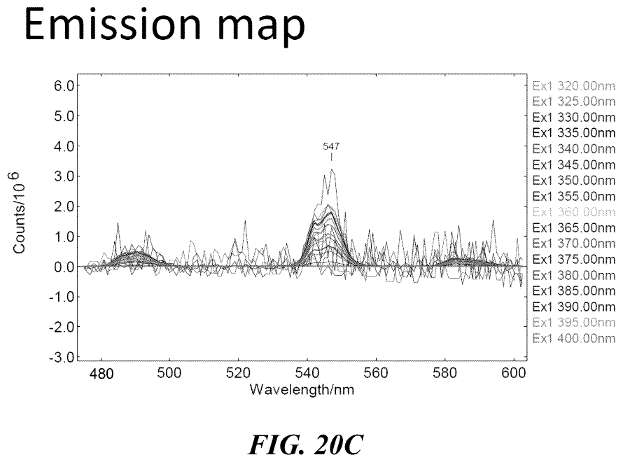

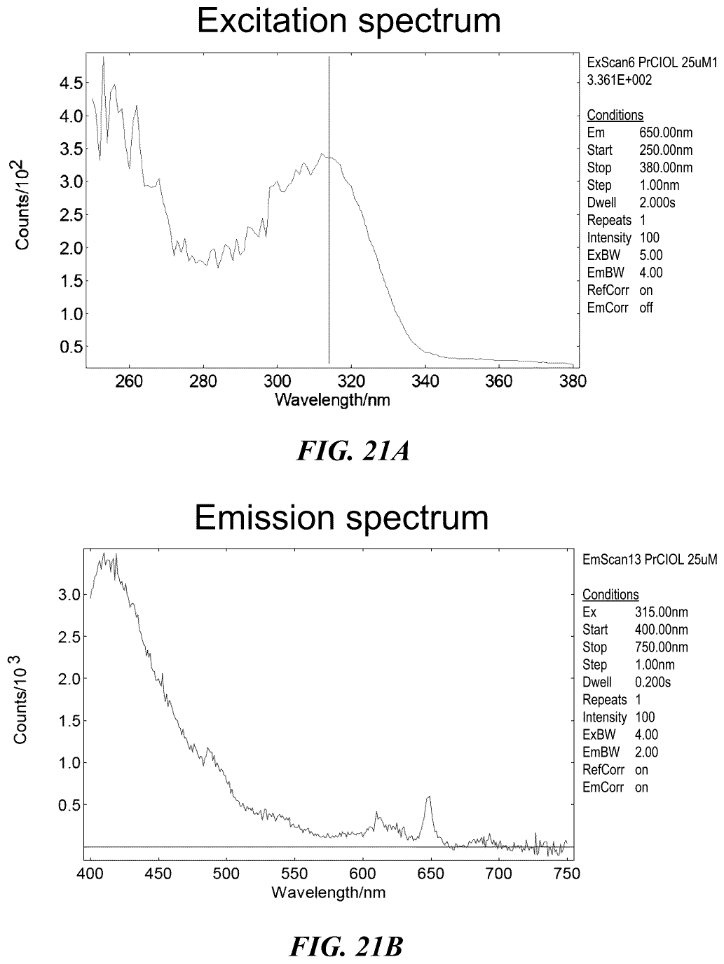

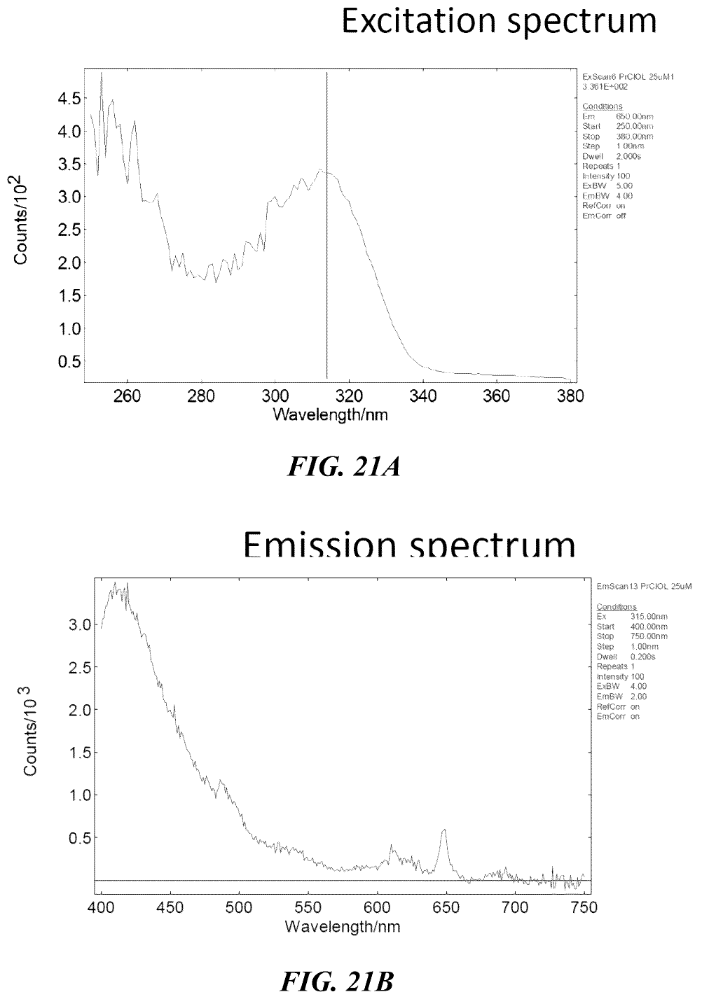

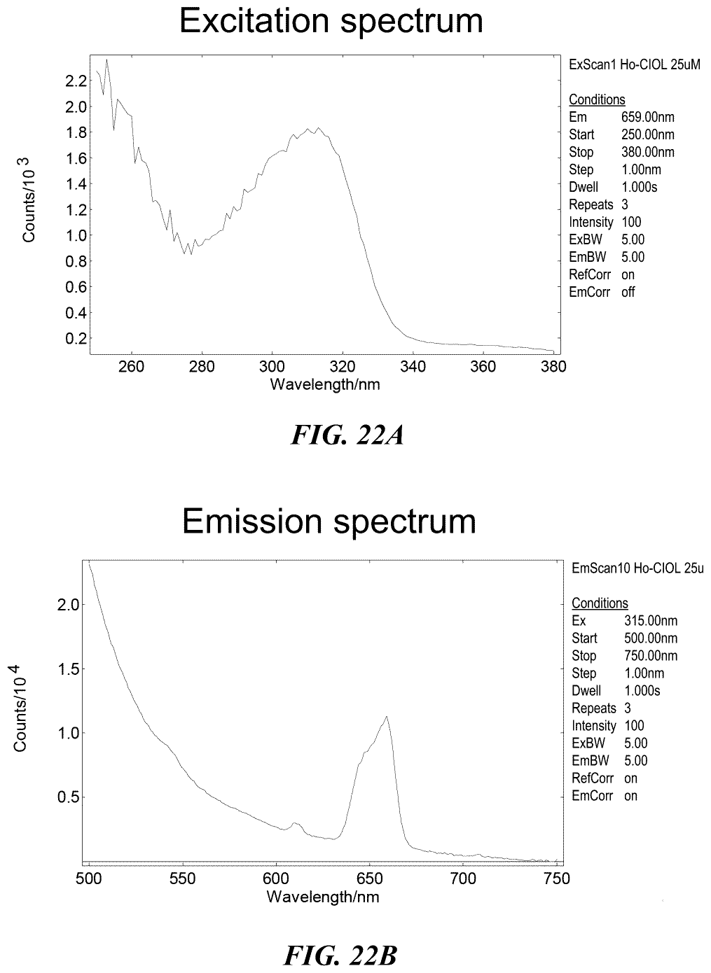

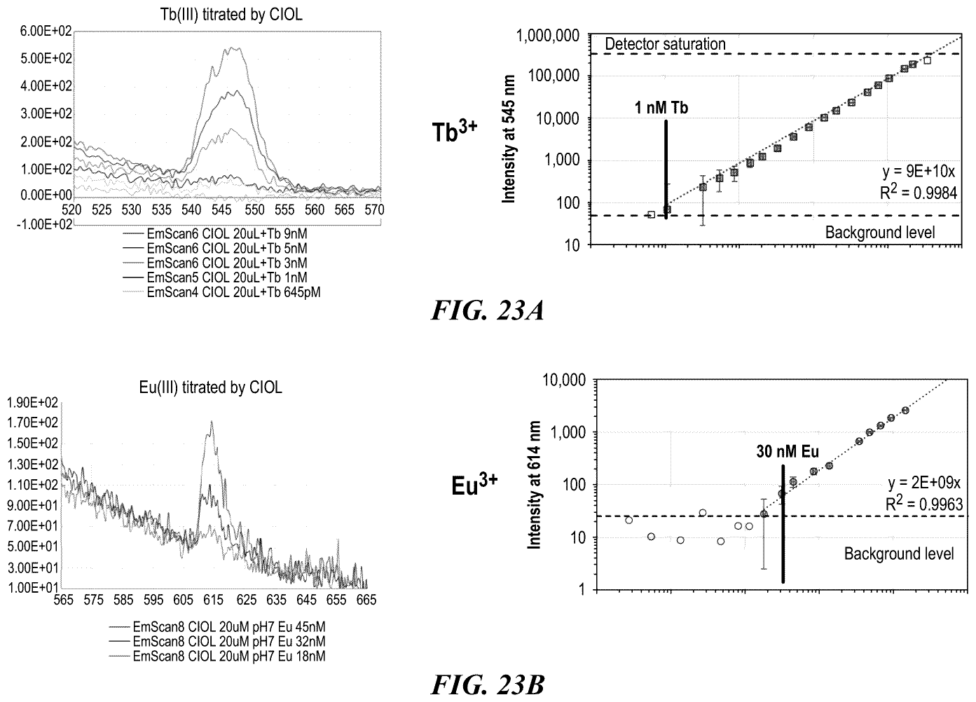

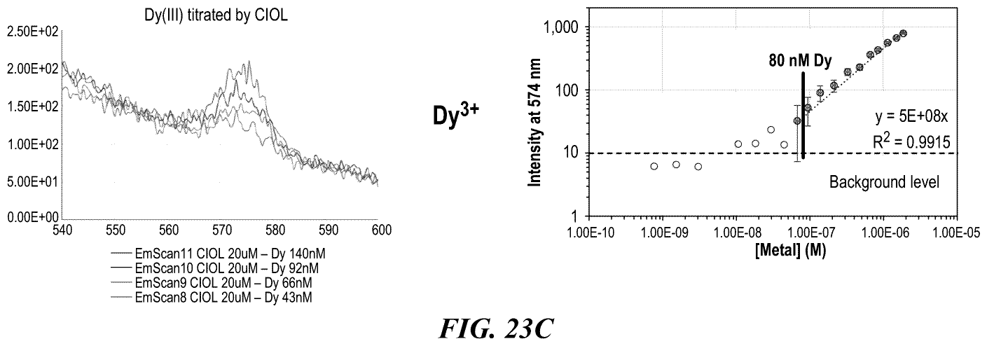

Detailed fluorescence measurements (using a FLS1000 fluorescence spectrometer), including emission spectra, excitation spectra, and fluorescence lifetimes for samples containing the ligand ciclopirox with REEs are shown in FIGS. 17A-17D, 18A-18D, 19A-19D, 20A-20D, 21A-21B, and 22A-22B. Estimations of the limit of detection for Eu with ciclopirox, Tb with ciclopirox, or Dy with ciclopirox (all using a FLS1000 fluorescence spectrometer) are shown in FIGS. 23A, 23B, and 23C, respectively.

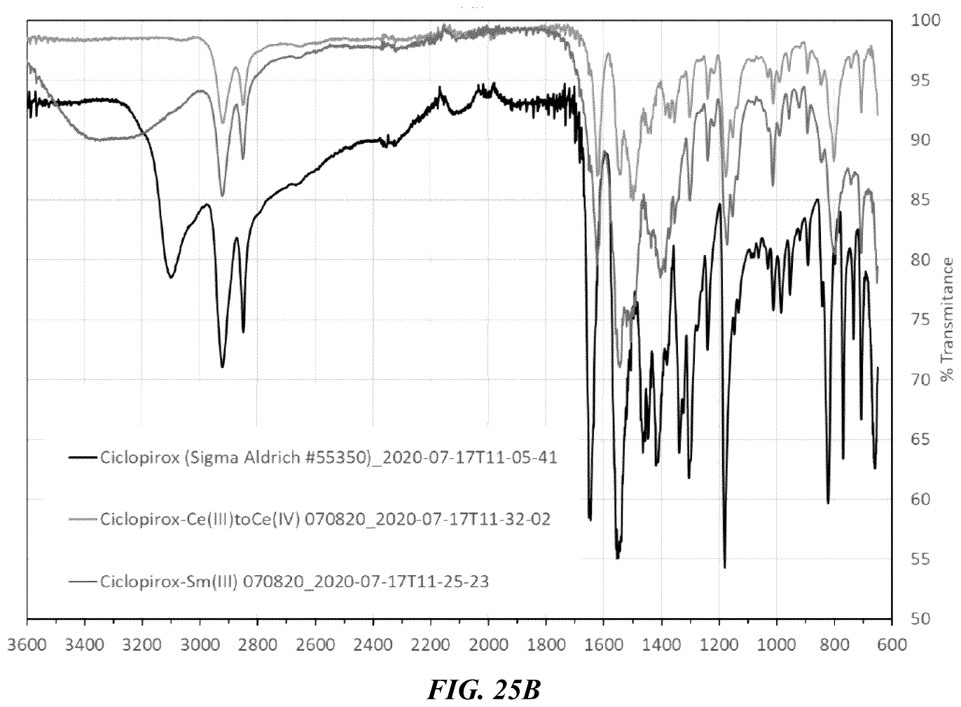

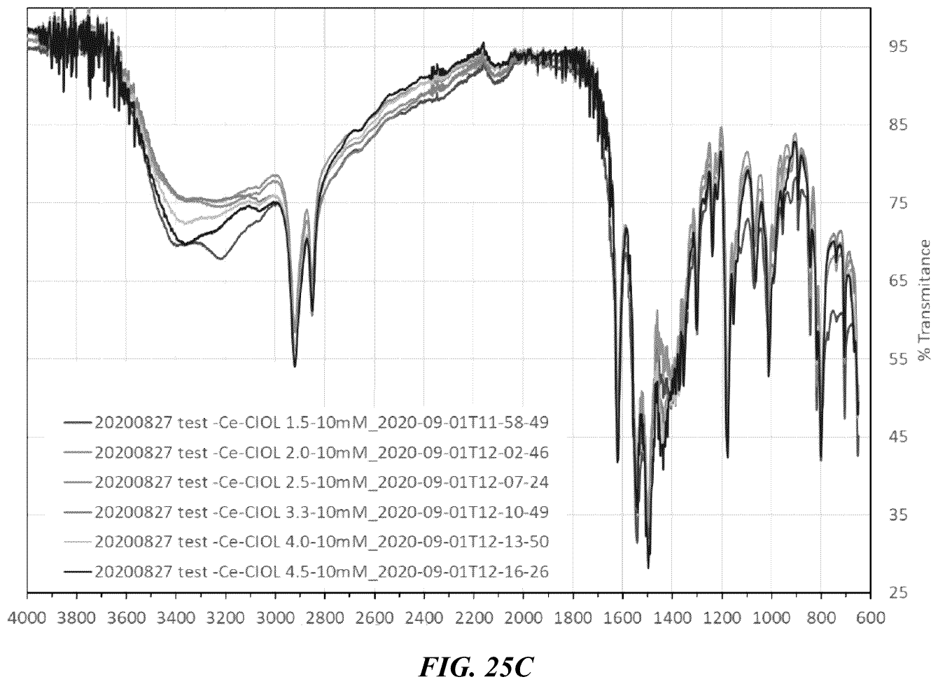

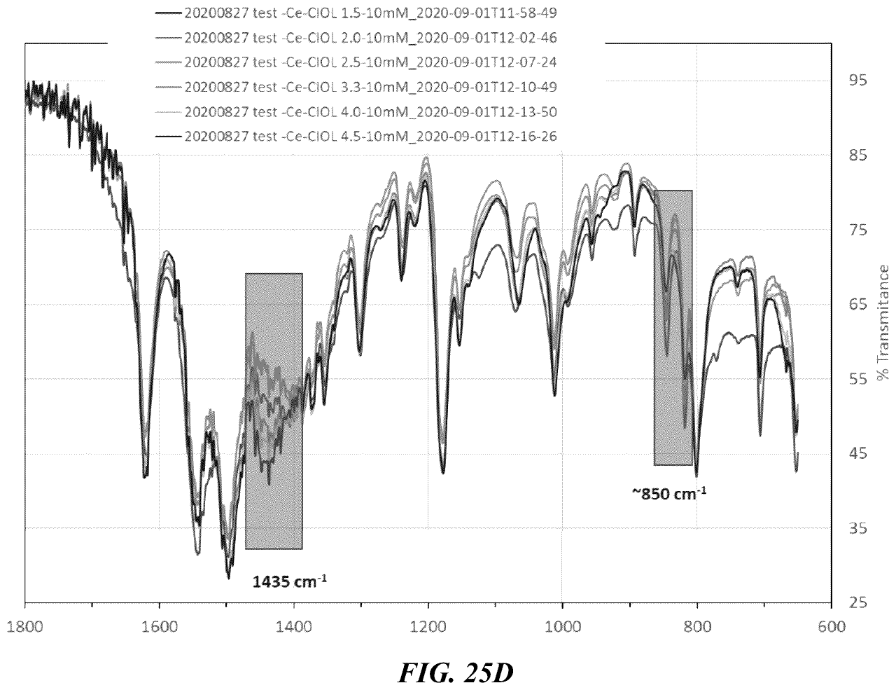

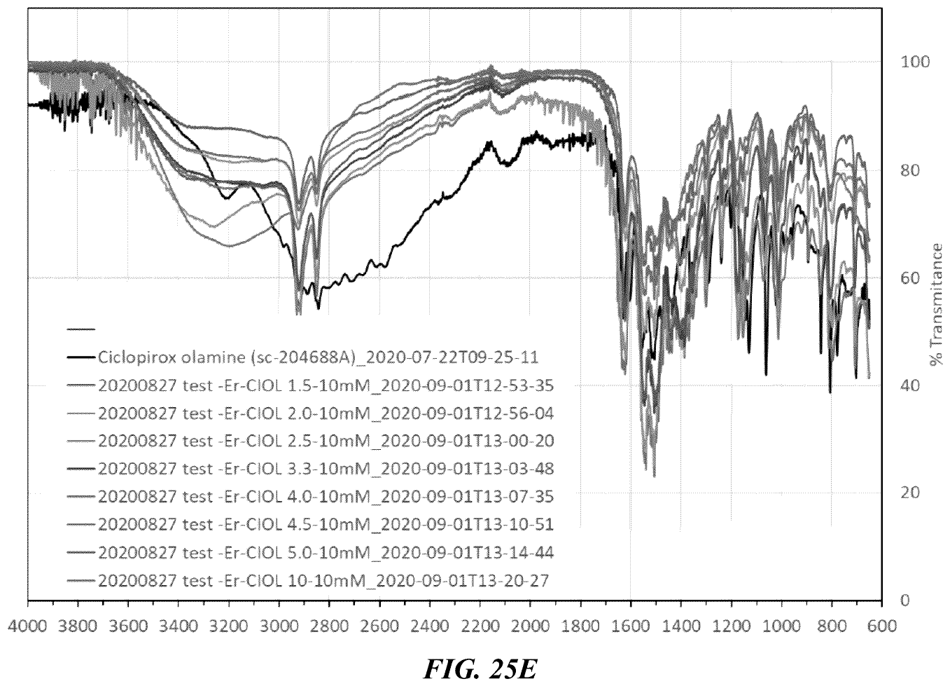

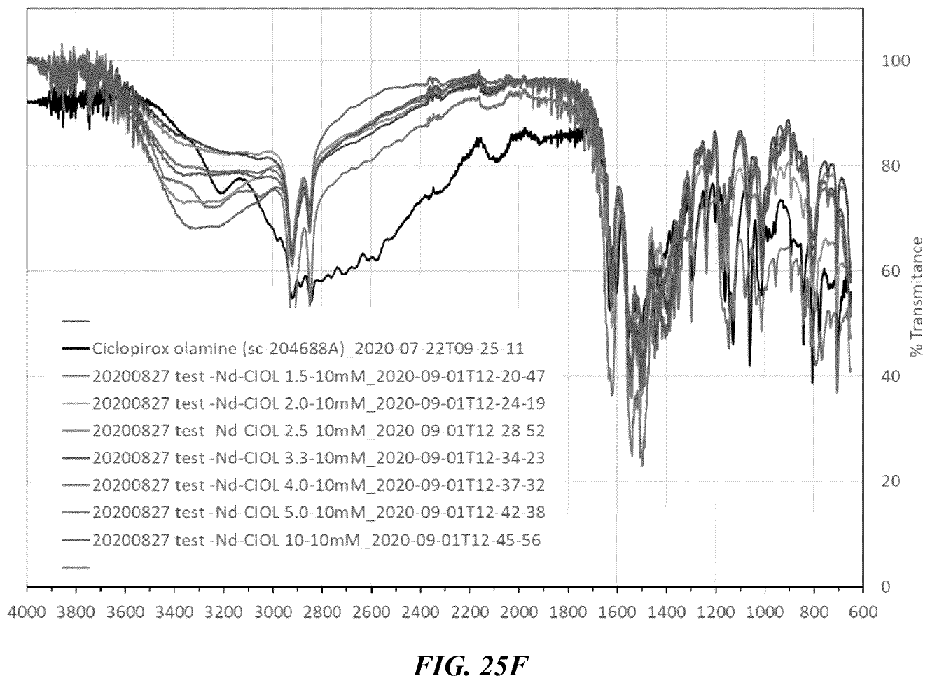

Fourier-Transform Infra-Red (FTIR) spectra of complexes of the molecules ciclopirox and piroctone with REEs (Ce⁴⁺, Eu³⁺, Sm³⁺, Nd³⁺, Er³⁺) were also performed using an Agilent FTIR Cary360 (FIGS. 25A-25F).

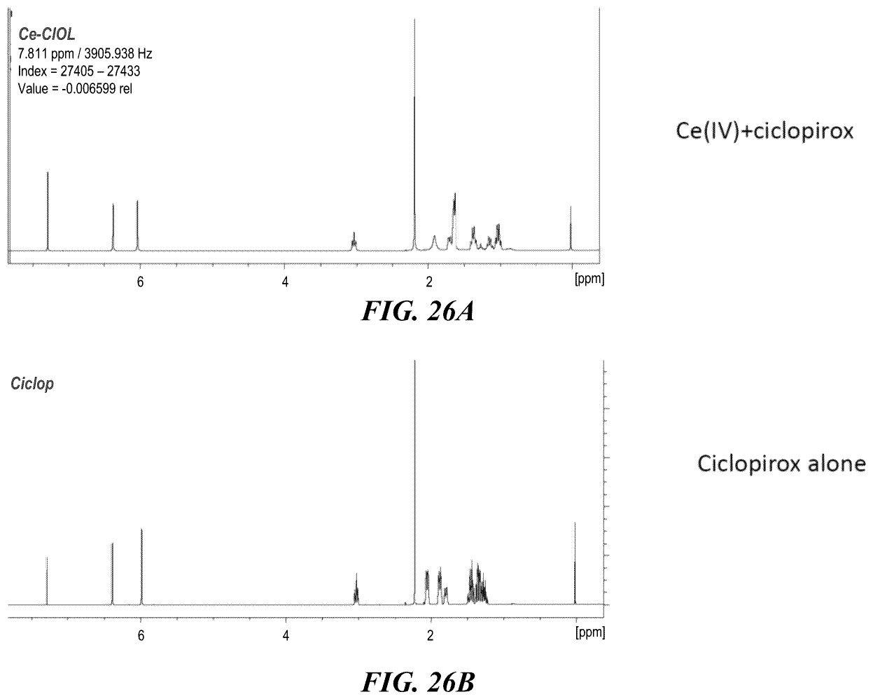

Further, nuclear magnetic resonance (1H NMR) spectra of the molecule ciclopirox alone and in the presence of the rare earth element cerium (IV), showed formation of a complex. FIGS. 26A-26B

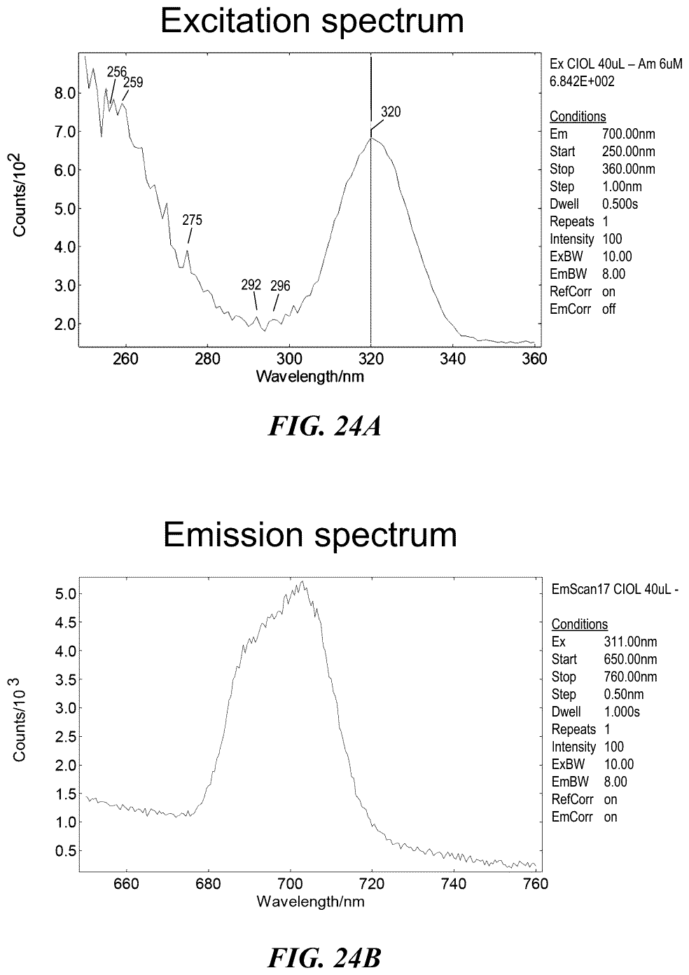

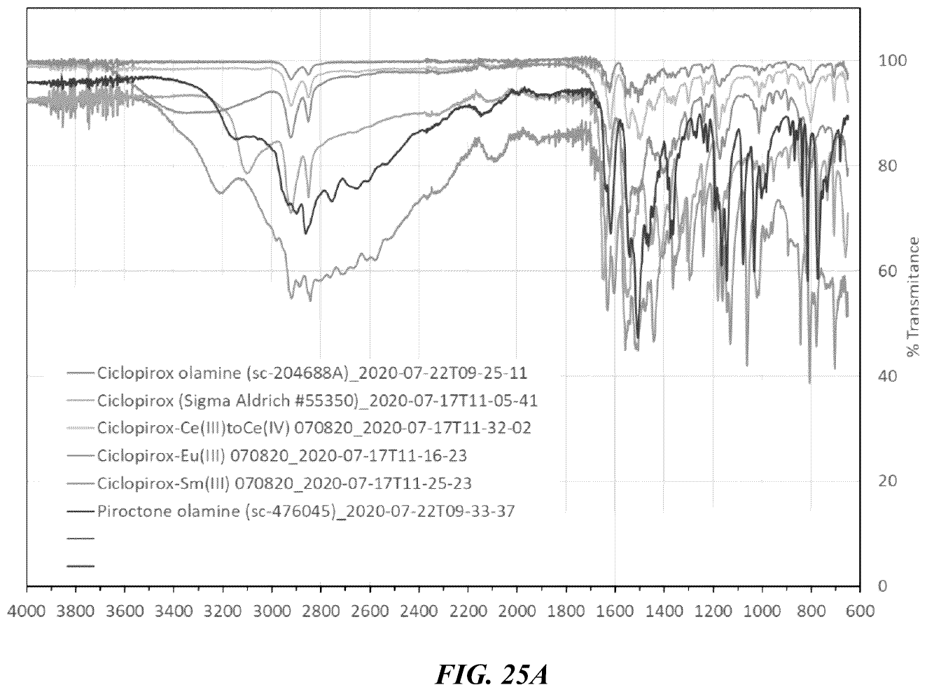

Actinide Samples. Tests performed with ciclopirox and the actinide element americium (Am³⁺) using a FLS1000 fluorescence spectrometer showed that ciclopirox also renders the americium ion luminescent. See FIGS. 24A-24B. It is noted that americium alone is not luminescent under the conditions tested, thus ciclopirox (or similar molecules) could be used for detecting americium.

### Example 2: Influence of Organic Chelators on Radionuclides

In this project, the initial goal was to study the influence of small organic chelators on the geochemical behavior of radionuclides, with a focus on Am³⁺ and Pu.

Chelator families of interest included naturally occurring Fe³⁺ ligands: 1,2-HOPO (a model for HOPO siderophores), catechin (model natural catechol), and ferrichrome A (a natural cyclic hydroxamate).

Previous chelators used mainly focused on complex home-made tetradentate, hexadentate or octadentate HOPO chelators (which are unlikely to be able to be used outside of the source lab), all were amide derivatives. On the other hand, although ciclopirox and piroctone have not previously been studied for actinide and lanthanide chemistry, both are inexpensive alternatives that are widely available, in numerous countries, making both an attractive option for the tests described herein. Other hydroxypyridinone chelators that may be used in accordance with the embodiments described herein include pyridoxatin, among others.

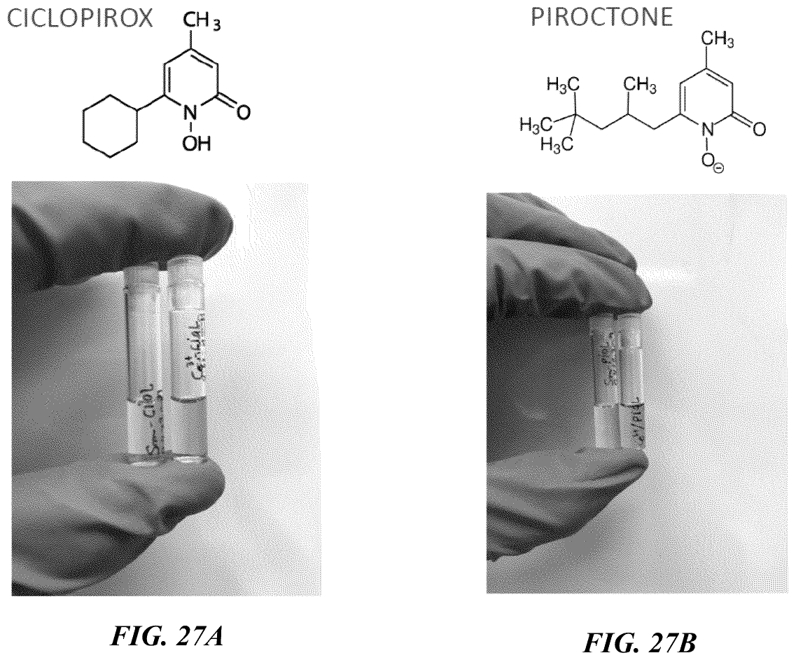

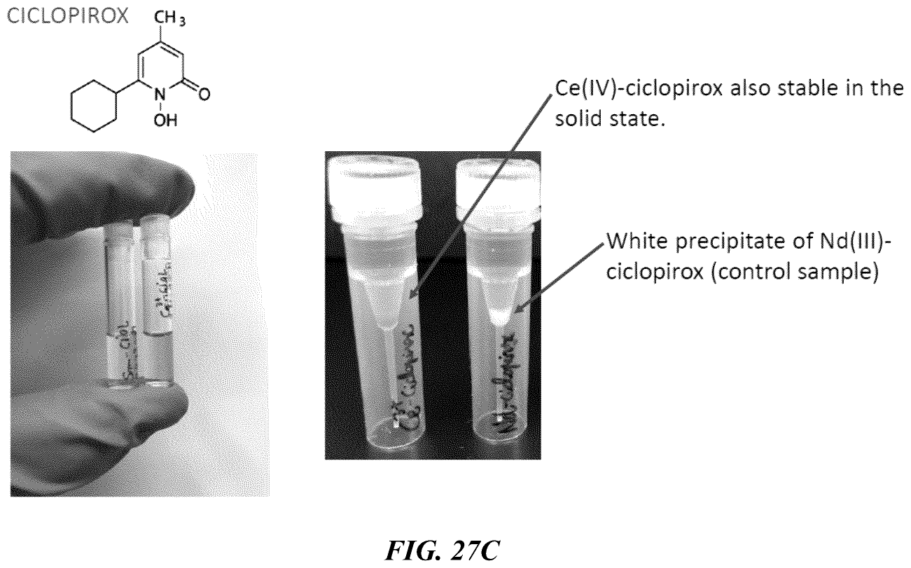

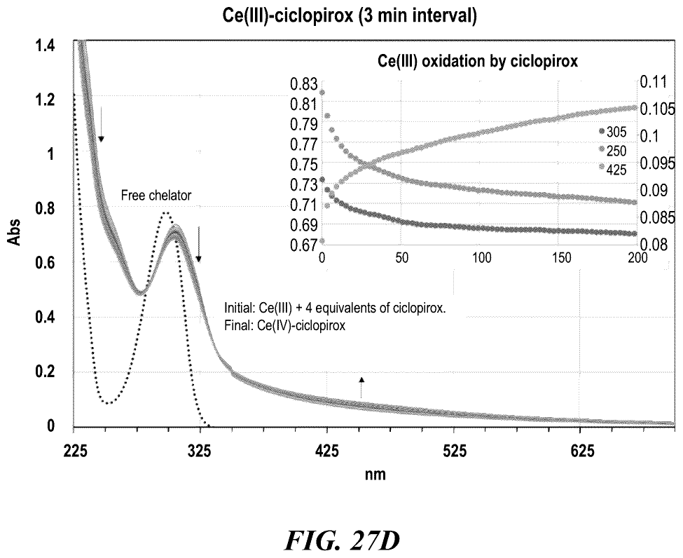

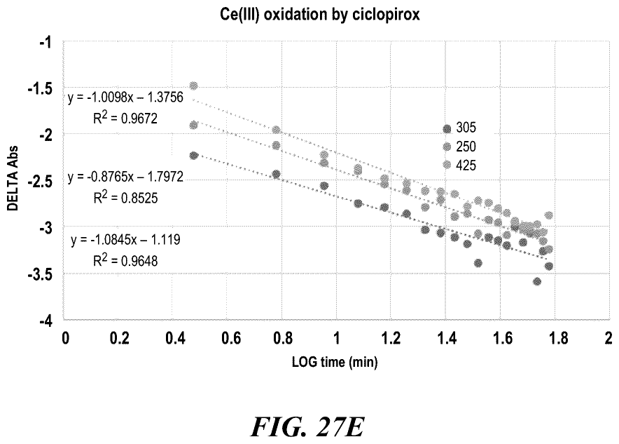

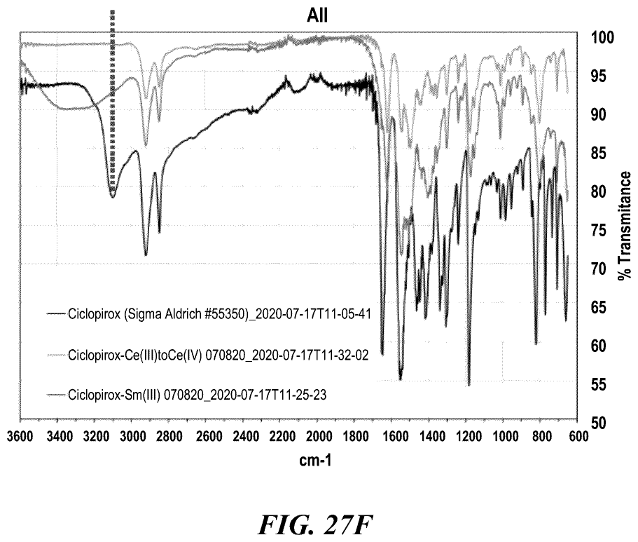

FIGS. 27A-27B show oxidation of Ce³⁺ to Ce⁴⁺ by ciclopirox and piroctone. The spontaneous oxidation of Ce³⁺ to Ce⁴⁺ by both ciclopirox and piroctone is visible as the samples from uncolored to orange. This strongly suggests that both chelators will strongly bind and stabilize Th⁴⁺, Bk⁴⁺, Pu⁴⁺ and Np⁴⁺. Ce(IV)-ciclopirox was also shown to be stable in the solid state as shown in FIG. 27C. The kinetics of the Ce(III) to Ce(IV) oxidation can be easily followed by UV-vis spectrophotometry, as shown in FIGS. 27D-27E. An FTIR of Ce(III) to Ce(IV)-ciclopirox, Sm(III)-ciclopirox, and unbound ciclopirox is also shown in FIG. 27F.

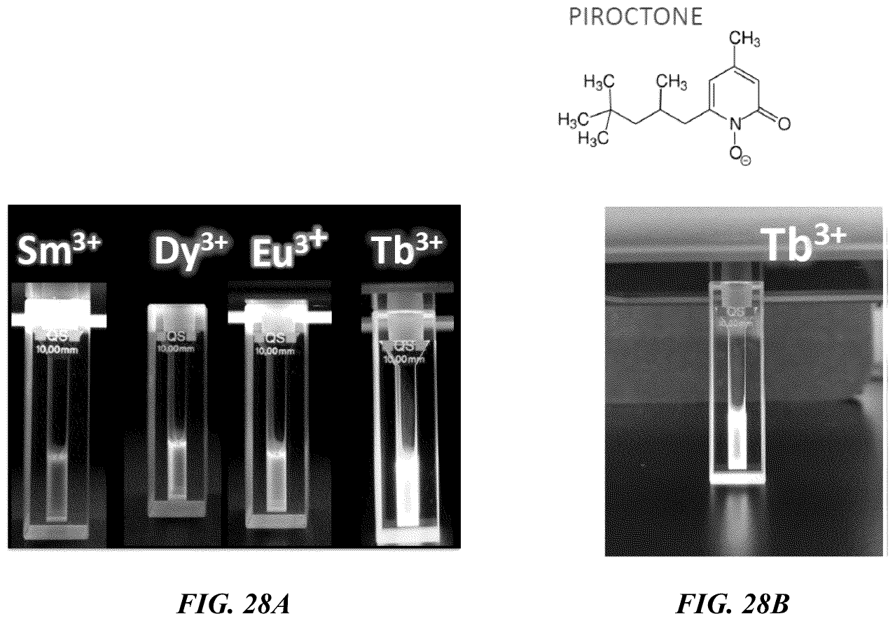

Luminescence properties of ciclopirox and piroctone with Ln(III) were also determined. In samples containing Ln³⁺ and 4 equivalents of piroctone are luminescent under UV-light (254 nm lamp). Ciclopirox and piroctone sensitize the luminescence of all four visible-emitting lanthanide ions (Sm³⁺, Dy³⁺, Eu³⁺, and Tb³⁺) as shown in FIGS. 28A-28B. This is very unexpected and rarely observed, and should sensitize Am³⁺ and Cm³⁺ as well.

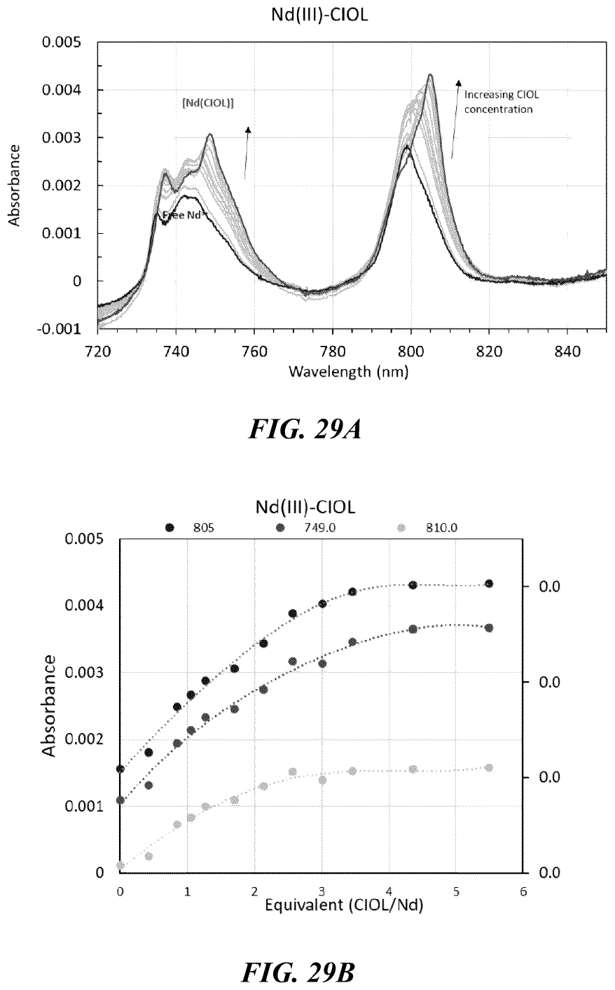

Complex stoichiometry with Ln(III) ions. The use of f-f absorbance bands of Nd(III) ions in the UV-vis domain to follow the metal-ligand complex formation. The results indicate the formation of a 1:3 complex. FIGS. 29A-29B. Since ciclopirox binds to Nd(III), it is very likely that ciclopirox sensitize the luminescence of Nd(III) also, which emits in the near-IR domain (900-1200 nm).

### Example 3: pH Dependence and Detection Limits of Ciclopirox and Piroctone

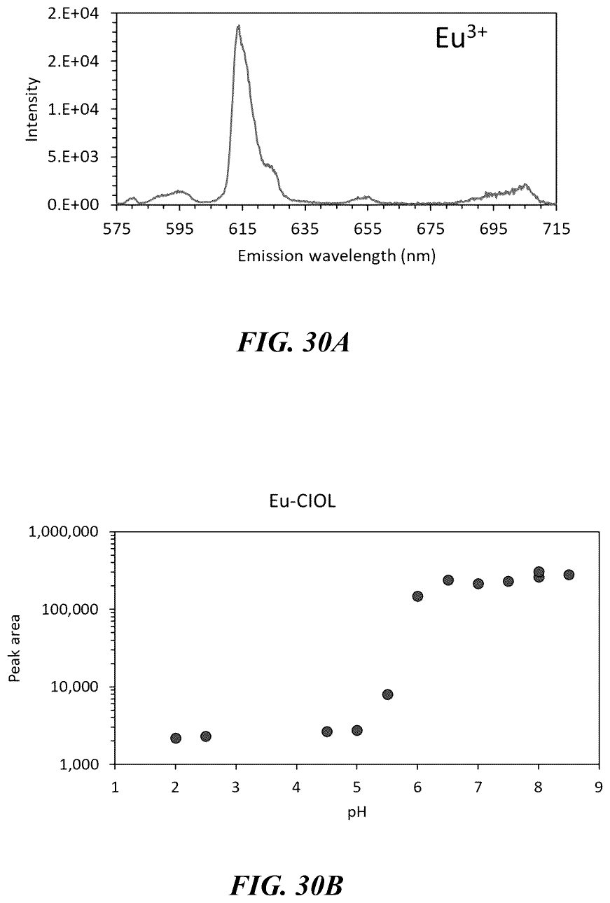

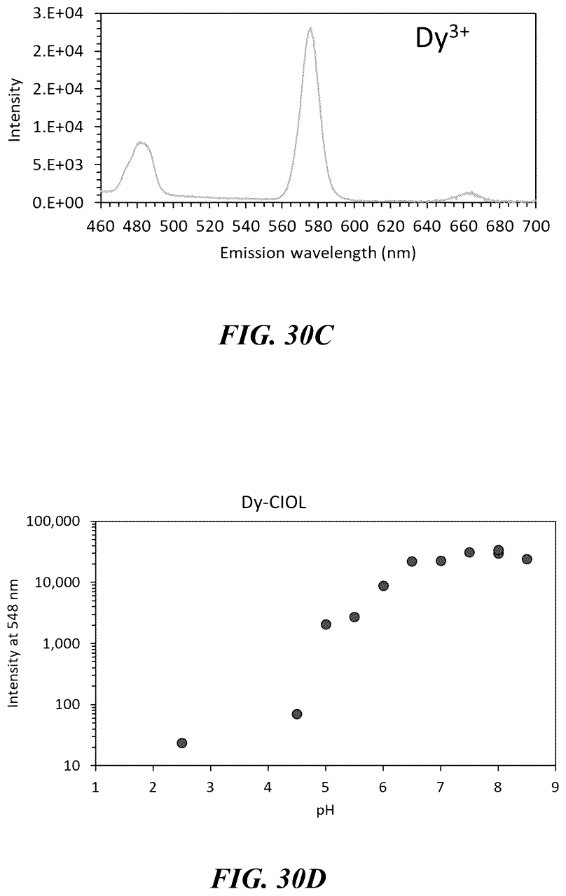

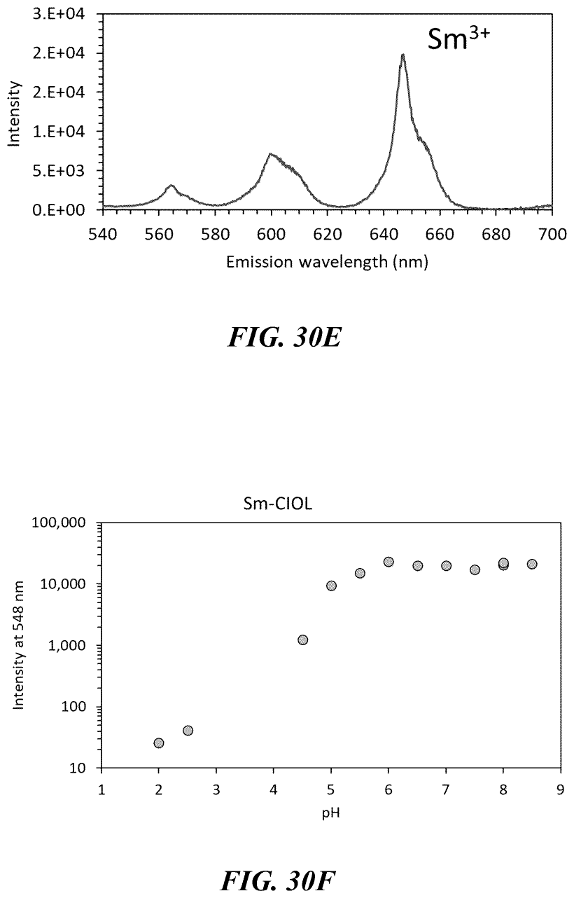

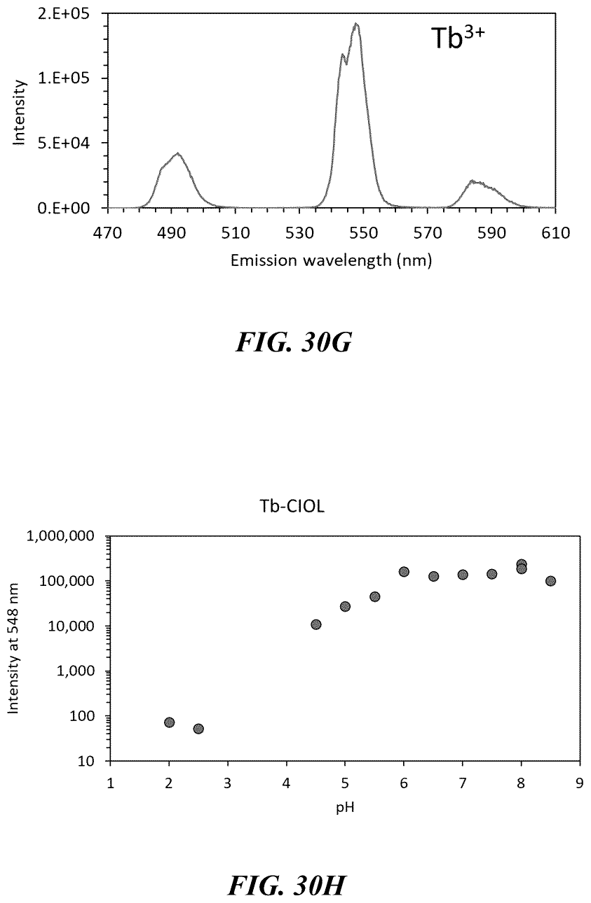

Ciclopirox. Fluorescence spectra of the rare earth elements Sm³⁺, Eu³⁺, Tb³⁺, and Dy³⁺ in the presence of ciclopirox (CIOL) were measured as a function of pH. FIGS. 30A-30H. The experiments were performed in blind test mode, with the technician not being given the formula of the compound. The results are consistent with prior observations, i.e., ciclopirox drastically enhances the luminescence of Sm³⁺, Eu³⁺, Tb³⁺, and Dy³⁺. Based on the results shown in FIGS. 30A-30H, it becomes evident that to analyze REE elements, an unknown sample could simply be diluted in a buffer, at pH 5 to 10, preferentially 7 to 9, containing the molecule ciclopirox.

Given the high fluoresncence properties of the REE complexes with ciclopirox, the initial sample could be diluted by a large dilution factor (e.g., 1,000 to 100,000), meaning any potentially interfering elements would become negligible.

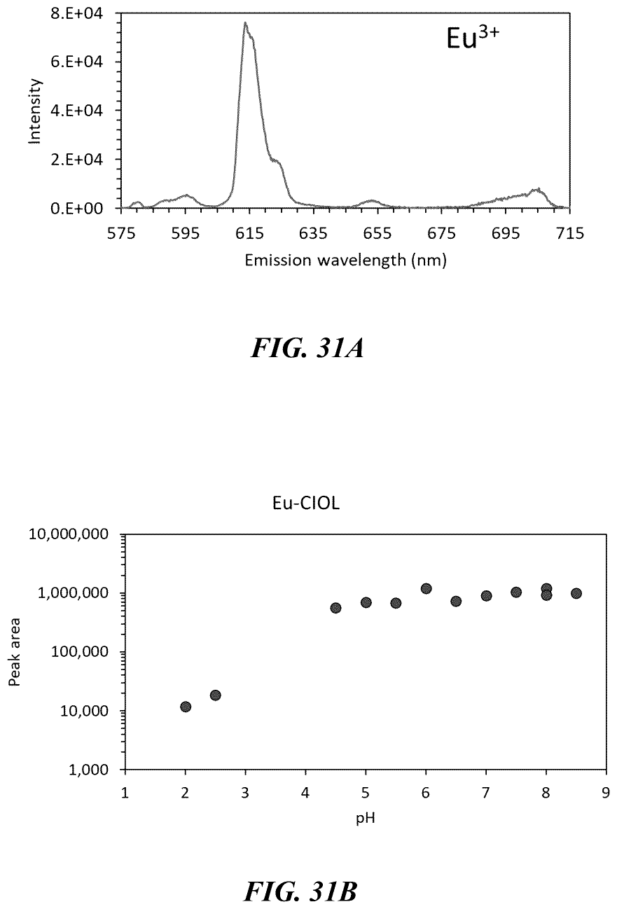

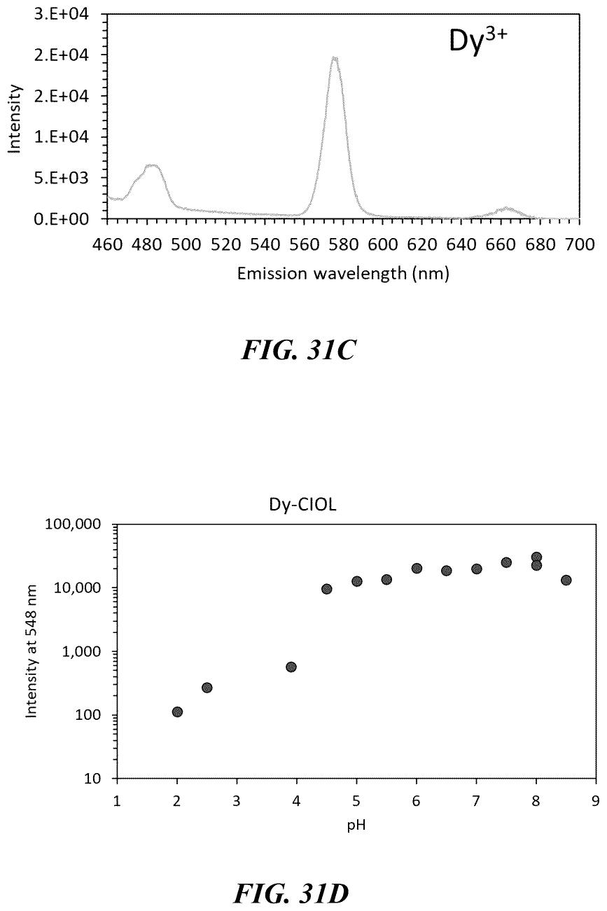

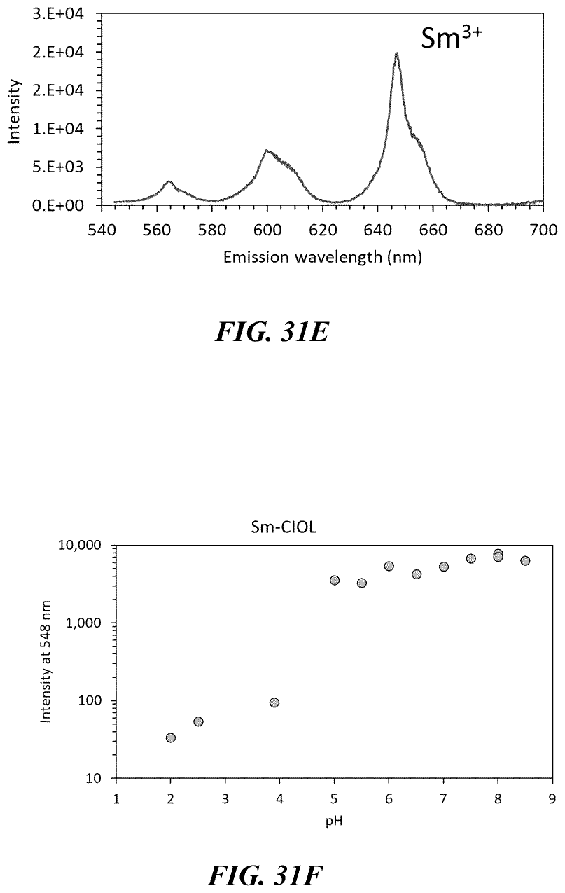

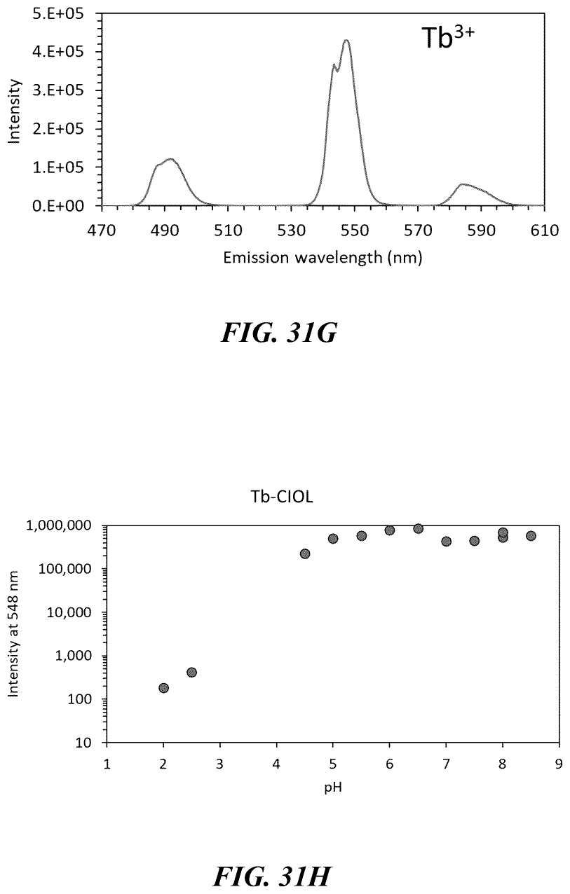

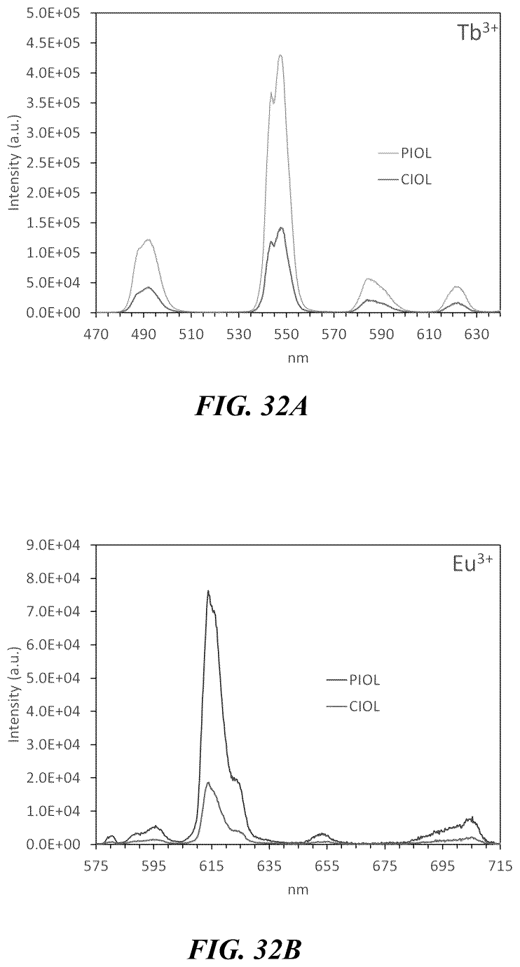

Piroctone. The same tests were performed with piroctone (PIOL), also in blind-test mode for the operator. The results also confirm the drastic enhancement of the luminescence properties of Sm³⁺, Eu³⁺, Tb³⁺, and Dy³⁺ in the presence of the piroctone. FIGS. 31A-31H. The spectral shapes and the pH dependence are substantially the same as those shown for ciclopirox, but the intensities are greater in the case of piroctone compared to ciclopirox. Although both molecules are able to detect REEs, the molecule piroctone (PIOL) is more efficient that the molecule ciclopirox (CIOL) at sensitizing the luminescence of REE ions. FIGS. 32A-32B. PIOL should therefore yield lower limits of detection.

### Example 4: Ciclopirox and Piroctone Sensitize Solid Deposits

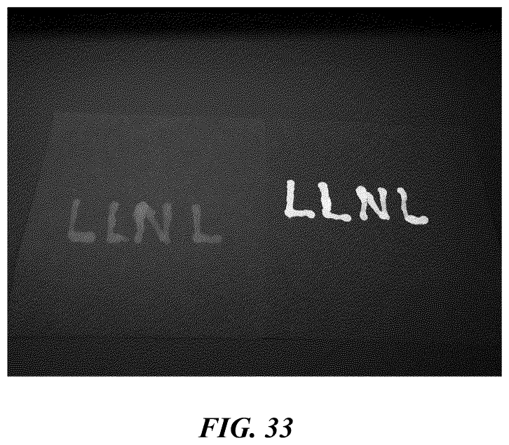

Solutions of Eu-CIOL or Tb-CIOL can also be deposited on solid materials, such as paper or wood. FIG. 33. Even once dried, the luminescence is retained, and samples can retain their luminescence for at least a year. Such systems could therefore be used in applications such as decorative items, anti-counterfeit inks, solid-state detection, etc.

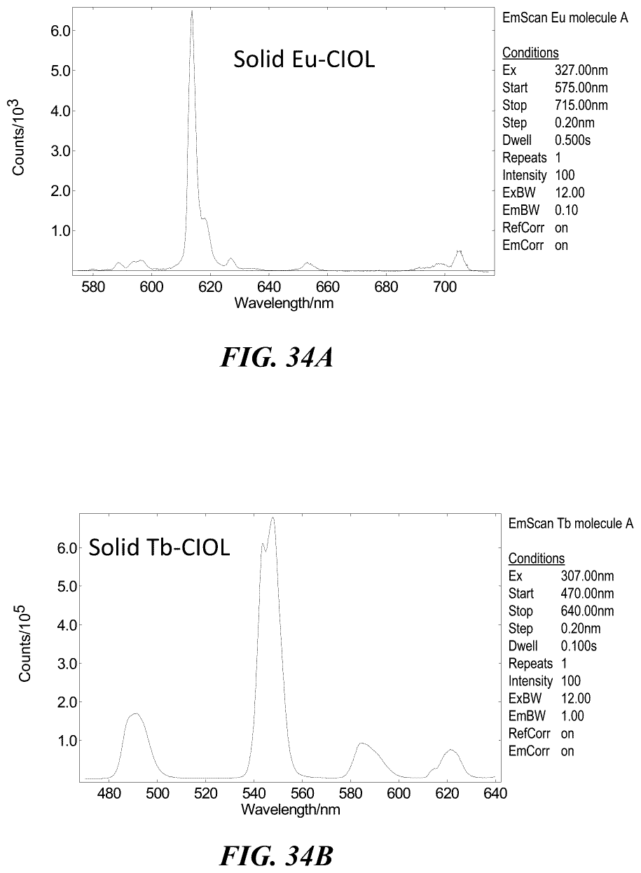

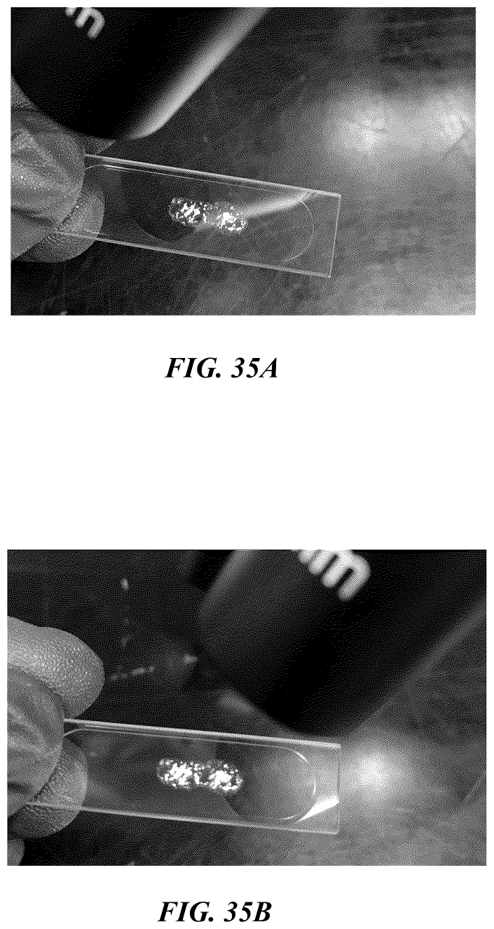

The fluorescence emission spectra of solid Eu-CIOL and Tb-CIOL (crystalline precipitate) were also measured. The results are consistent with the spectral fingerprints observed in solution. FIGS. 34A-34B, 35A-35B. The results confirm that the complexes retain their luminescence properties in the solid-state (crystalline and amorphous forms).

### Example 5: Ciclopirox and Piroctone Sensitize Radioactive Actinides

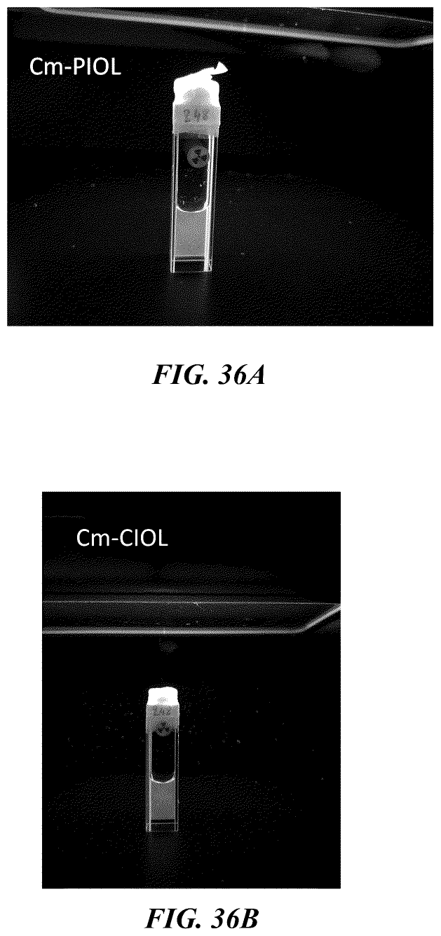

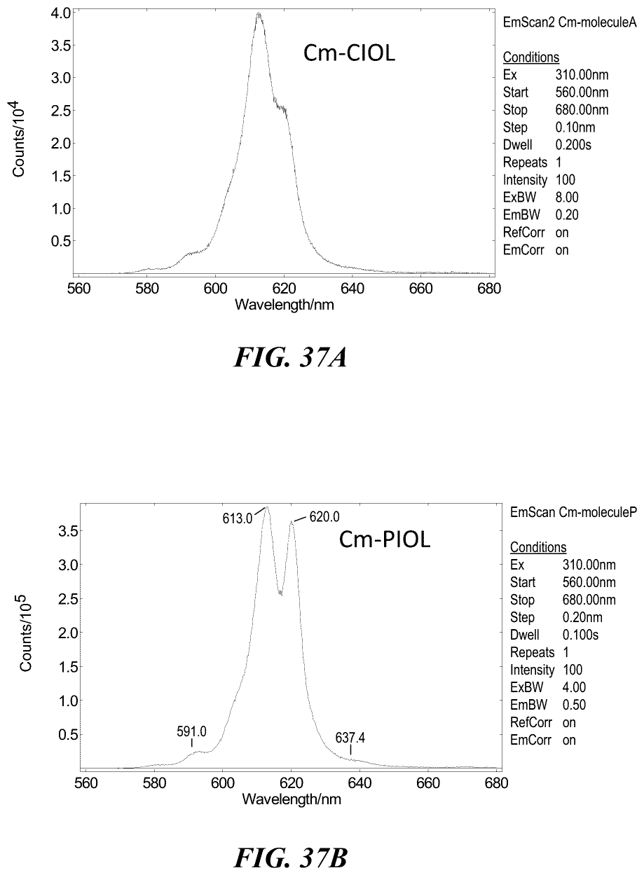

Experiments were performed with curium (ion in solution: Cm³⁺), a radioactive element belonging to the actinide series. The molecules CIOL and PIOL also render the Cm³⁺ ion highly luminescent. FIGS. 36A-36B. The fluorescence emission spectrum of Cm-CIOL and Cm-PIOL were measured, as shown in FIGS. 37A-37B. The limit of detection for curium in the presence of CIOL was also evaluated. FIGS. 38A-38C.

Based on these results, it is evident that curium can be detected at very low concentrations, via fluorescence, following dilution of the studied sample into a solution containing CIOL. In this example, the limit of detection is 0.5 nanoM (5E-10 mol/L, equivalent to ˜0.1 ppb) or lower. Based on the results mentioned previously, a similar behavior is expected with PIOL.

### Example 6: SUN-B, Tenellin, and Pyridoxatin

Additional experiments were performed to compare the potency of other molecules for rare earth detection (by luminescence) relative to ciclopirox and piroctone: SUN-B, tenellin, and pyridoxatin.

Samples containing Eu³⁺ or Tb³⁺ and the molecules of interest were analyzed via fluorescence spectroscopy. Piroctone, ciclopirox, and pyridoxatin are able to sensitize the fluorescence of both Eu³⁺ and Tb³⁺. However, SUN-B and tenellin did not yield fluorescent samples and therefore would not be used for fluorescence detection of rare earth or actinide elements. The potency of the molecules for metal detection is as follows:

### Piroctone >Ciclopirox>Pyridoxatin.

The cost of those molecules is as follows:

### Ciclopirox>Piroctone>>>>>Pyridoxatin.

In certain embodiments, ciclopirox and piroctone would be used preferentially to reduce costs.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A composition comprising:
a light-harvesting molecule capable of producing luminescence upon interaction with a rare earth element and/or an actinide element, the light-harvesting molecule comprising one hydroxypyridinone (HOPO) group having the structure of formula (I) or (II): 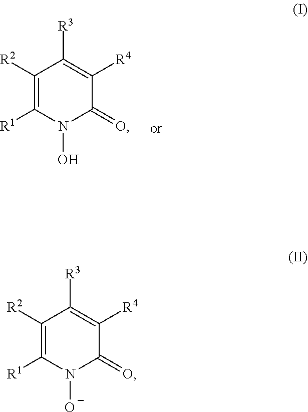
or a salt, solvate, tautomer, or stereoisomer thereof,
wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; and
a buffer;
wherein the composition has a pH between about 6 to about 11.

2. The composition of claim 1, wherein the light-harvesting molecule lacks an amide group.

3. The composition of claim 1 or 2, wherein the composition is in the form of an aqueous solution.

4. The composition of claim 1 or 2, wherein the composition is in the form of a solution, which further comprises water or an organic solvent.

5. The composition of claim 4, wherein the organic solvent is at least one selected from the group consisting of an alcohol, acetonitrile, dimethylformamide (DMF), and dimethyl sulfoxide (DMSO).

6. The composition of any one of claims 1-5, wherein the light-harvesting molecule comprises one hydroxypyridinone (HOPO) group having the structure of formula (I), or a salt, solvate, tautomer, or stereoisomer thereof.

7. The composition of any one of claims 1-5, wherein the light-harvesting molecule comprises one hydroxypyridinone (HOPO) group having the structure of formula (II), or a salt, solvate, tautomer, or stereoisomer thereof.

8. The composition of any one of claims 1-7, wherein:
R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms;
R² is hydrogen;
R³ is an alkyl group having from 1 to 6 carbon atoms; and
R⁴ is hydrogen.

9. The composition of any one of claims 1-8, wherein R³ is a methyl group (—CH₃).

10. The composition of any one of claims 1-9, wherein R¹ is an alkyl group having between 1 to 10 carbon atoms.

11. The composition of any one of claims 1-10, wherein R¹ is an alkyl group having 8 carbon atoms.

12. The composition of any one of claims 1-11, wherein R¹ is —CH₂CH(CH₃)CH₂C(CH₃)₃.

13. The composition of any one of claims 1-12, wherein the light-harvesting molecule comprises the structure of piroctone or a salt thereof:

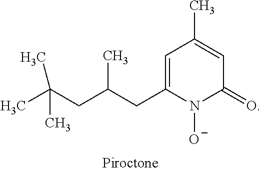 [SMILES: Cc1cc(CC(C)CC(C)(C)C)n([O-])c(=O)c1]

14. The composition of claim 13, wherein piroctone is in the form of piroctone olamine.

15. The composition of any one of claims 1-9, wherein R¹ is a cycloalkyl or substituted cycloalkyl group having between 3 and 8 carbon atoms.

16. The composition of any one of claims 1-9 and 15, wherein R¹ is a cycloalkyl or substituted cycloalkyl group having 6 carbon atoms.

17. The composition of any one of claims 1-9 and 15-16, wherein R¹ is cyclohexyl.

18. The composition of any one of claims 1-9 and 15-17, wherein the light-harvesting molecule comprises the structure of ciclopirox or a salt thereof:

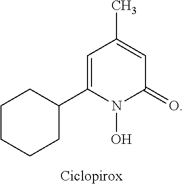

19. The composition of claim 18, wherein ciclopirox is in the form of ciclopirox olamine.

20. The composition of any one of claims 1-7, wherein:
R¹ is hydrogen;
R² is hydrogen;
R³ is a hydroxy group (—OH); and
R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

21. The composition of any one of claims 1-7 and 20, wherein R⁴ is a cycloalkyl or substituted cycloalkyl group having between 5 and 14 carbon atoms.

22. The composition of any one of claims 1-7 and 20-21, wherein R⁴ is a cycloalkyl or substituted cycloalkyl group having 10 carbon atoms.

23. The composition of any one of claims 1-7 and 20-22, wherein R⁴ is

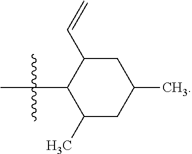 [SMILES: *C1C(C)CC(C)CC1C=C]

24. The composition of any one of claims 1-7 and 20-23, wherein the light-harvesting molecule comprises the structure of pyridoxatin, or an enantiomer, or a tautomer thereof:

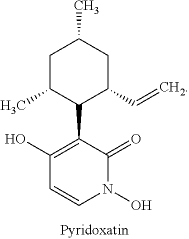

25. The composition of any one of claims 1-24, wherein the rare earth element is at least one selected from the group consisting of Sm, Eu, Tb, Dy, Pr, and Ho.

26. The composition of any one of claims 1-25, wherein the actinide element is Cm and/or Am.

27. A composition comprising:
a light-harvesting molecule capable of producing luminescence upon interaction with a rare earth element and/or an actinide element, the light-harvesting molecule comprising one hydroxypyridinone (HOPO) group having the structure of formula (I) or (II): 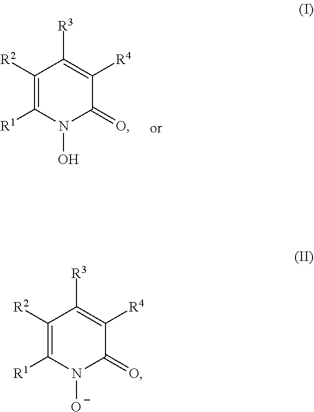
or a salt, solvate, tautomer, or stereoisomer thereof,
wherein each of R¹-R⁴ are independently selected from a hydrogen, a hydroxy group, or a hydrocarbon group, wherein the hydrocarbon group is an alkyl group having from 1 to 20 carbon atoms, a substituted alkyl group having from 1 to 20 carbon atoms, a cycloalkyl group having from 3 to 20 carbon atoms, or a substituted cycloalkyl group having from 3 to 20 carbon atoms; and
a buffer;
wherein the composition has a pH between about 6 to about 11.

28. The composition of claim 27, wherein R¹ is —CH₂CH(CH₃)CH₂C(CH₃)₃, cyclohexyl, or hydrogen, R² is hydrogen, R³ is a methyl group (—CH₃) or a hydroxy group, and R⁴ is hydrogen or

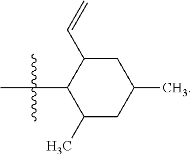 [SMILES: *C1C(C)CC(C)CC1C=C]

29. An assay system comprising the composition of any one of claims 1-28.

30. An assay system comprising a light-harvesting molecule immobilized on an assay test strip, wherein the light-harvesting molecule is capable of producing luminescence upon interaction with a rare earth element and/or an actinide element, the light-harvesting molecule comprises one hydroxypyridinone (HOPO) group having the structure of formula (I) or (II): 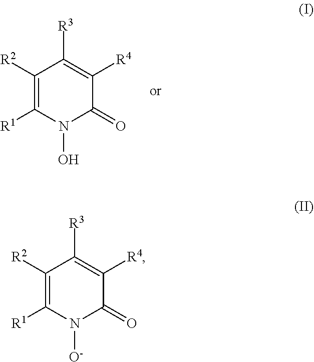
or a salt, solvate, tautomer, or stereoisomer thereof,
wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; and
the light-harvesting molecule is present in a composition comprising a buffer, wherein the composition has a pH between about 6 to about 11.

31. The assay system of claim 30, wherein the light-harvesting molecule lacks an amide group.

32. The assay system of claim 30 or 31, wherein the composition is in the form of an aqueous solution.

33. The assay system of claim 30 or 31, wherein the composition is in the form of a solution, which further comprises water or an organic solvent.

34. The assay system of claim 33, wherein the organic solvent is at least one selected from the group consisting of an alcohol, acetonitrile, dimethylformamide (DMF), and dimethyl sulfoxide (DMSO).

35. The assay system of any one of claims 30-34, wherein the light-harvesting molecule comprises one hydroxypyridinone (HOPO) group having the structure of formula (I), or a salt, solvate, tautomer, or stereoisomer thereof.

36. The assay system of any one of claims 30-34, wherein the light-harvesting molecule comprises one hydroxypyridinone (HOPO) group having the structure of formula (II), or a salt, solvate, tautomer, or stereoisomer thereof.

37. The assay system of any one of claims 30-36, wherein:
R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms;
R² is hydrogen;
R³ is an alkyl group having from 1 to 6 carbon atoms; and
R⁴ is hydrogen.

38. The assay system of any one of claims 30-37, wherein R³ is a methyl group (—CH₃).

39. The assay system of any one of claims 30-38, wherein R¹ is an alkyl group having between 1 to 10 carbon atoms.

40. The assay system of any one of claims 30-39, wherein R¹ is an alkyl group having 8 carbon atoms.

41. The assay system of any one of claims 30-40, wherein R¹ is —CH₂CH(CH₃)CH₂C(CH₃)₃.

42. The assay system of any one of claims 30-41, wherein the light-harvesting molecule comprises the structure of piroctone or a salt thereof:

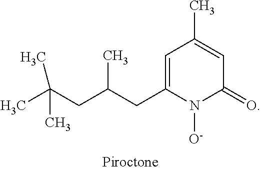 [SMILES: Cc1cc(CC(C)CC(C)(C)C)n([O-])c(=O)c1]

43. The assay system of claim 42, wherein piroctone is in the form of piroctone olamine.

44. The assay system of any one of claims 30-38, wherein R¹ is a cycloalkyl or substituted cycloalkyl group having between 3 and 8 carbon atoms.

45. The assay system of any one of claims 30-38 and 44, wherein R¹ is a cycloalkyl or substituted cycloalkyl group having 6 carbon atoms.

46. The assay system of any one of claims 30-38 and 44-45, wherein R¹ is cyclohexyl.

47. The assay system of any one of claims 30-38 and 44-46, wherein the light-harvesting molecule comprises the structure of ciclopirox or a salt thereof:

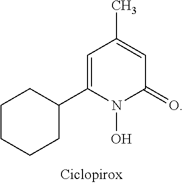

48. The assay system of claim 47, wherein ciclopirox is in the form of ciclopirox olamine.

49. The assay system of any one of claims 30-36, wherein:
R¹ is hydrogen;
R² is hydrogen;
R³ is a hydroxy group (—OH); and
R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

50. The assay system of any one of claims 30-36 and 49, wherein R⁴ is a cycloalkyl or substituted cycloalkyl group having between 5 and 14 carbon atoms.

51. The assay system of any one of claims 30-36 and 49-50, wherein R⁴ is a cycloalkyl or substituted cycloalkyl group having 10 carbon atoms.

52. The assay system of any one of claims 30-36 and 49-51, wherein R⁴ is

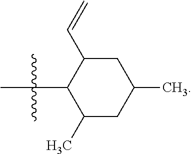 [SMILES: *C1C(C)CC(C)CC1C=C]

53. The assay system of any one of claims 30-36 and 49-52, wherein the light-harvesting molecule comprises the structure of pyridoxatin, or an enantiomer, or a tautomer thereof:

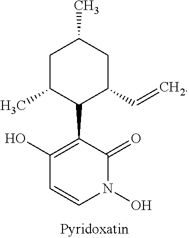

54. The assay system of any one of claims 30-53, wherein the rare earth element is at least one selected from the group consisting of Sm, Eu, Tb, Dy, Pr, and Ho.

55. The assay system of any one of claims 30-54, wherein the actinide element is Cm and/or Am.

56. A kit comprising:
a reagent comprising the composition of any one of claims 1-28; and
one or more containers for combining an aliquot of the reagent with a test sample.

57. A kit comprising:
a light-harvesting molecule immobilized on an assay test strip, wherein the light-harvesting molecule is capable of producing luminescence upon interaction with a rare earth element and/or an actinide element, the light-harvesting molecule comprises one hydroxypyridinone (HOPO) group having the structure of formula (I) or (II): 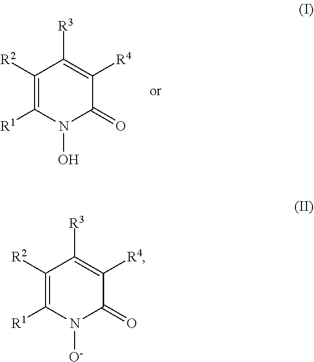
or a salt, solvate, tautomer, or stereoisomer thereof,
wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms; and
optionally a solution comprising a buffer for dissolving a test sample comprising a rare earth element and/or an actinide element.

58. The kit of claim 57, wherein the solution is present, wherein the kit further comprises one or more containers for combining an aliquot of the solution with the test sample.

59. The kit of claim 57 or 58, wherein the light-harvesting molecule lacks an amide group.

60. The kit of any one of claims 57-59, wherein the solution is present, and the solution is an aqueous solution.

61. The kit of any one of claims 57-59, wherein the solution is present, and the solution comprises water or an organic solvent.

62. The kit of claim 61, wherein the organic solvent is at least one selected from the group consisting of an alcohol, acetonitrile, dimethylformamide (DMF), and dimethyl sulfoxide (DMSO).

63. The kit of any one of claims 57-62, wherein the light-harvesting molecule comprises one hydroxypyridinone (HOPO) group having the structure of formula (I), or a salt, solvate, tautomer, or stereoisomer thereof.

64. The kit of any one of claims 57-62, wherein the light-harvesting molecule comprises one hydroxypyridinone (HOPO) group having the structure of formula (II), or a salt, solvate, tautomer, or stereoisomer thereof.

65. The kit of any one of claims 57-64, wherein:
R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms;
R² is hydrogen;
R³ is an alkyl group having from 1 to 6 carbon atoms; and
R⁴ is hydrogen.

66. The kit of any one of claims 57-65, wherein R³ is a methyl group (—CH₃).

67. The kit of any one of claims 57-66, wherein R¹ is an alkyl group having between 1 to 10 carbon atoms.

68. The kit of any one of claims 57-67, wherein R¹ is an alkyl group having 8 carbon atoms.

69. The kit of any one of claims 57-68, wherein R¹ is —CH₂CH(CH₃)CH₂C(CH₃)₃.

70. The kit of any one of claims 57-69, wherein the light-harvesting molecule comprises the structure of piroctone or a salt thereof:

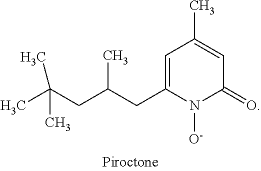 [SMILES: Cc1cc(CC(C)CC(C)(C)C)n([O-])c(=O)c1]

71. The kit of claim 70, wherein piroctone is in the form of piroctone olamine.

72. The kit of any one of claims 57-66, wherein R¹ is a cycloalkyl or substituted cycloalkyl group having between 3 and 8 carbon atoms.

73. The kit of any one of claims 57-66 and 72, wherein R¹ is a cycloalkyl or substituted cycloalkyl group having 6 carbon atoms.

74. The kit of any one of claims 57-66 and 72-73, wherein R¹ is cyclohexyl.

75. The kit of any one of claims 57-66 and 72-74, wherein the light-harvesting molecule comprises the structure of ciclopirox or a salt thereof:

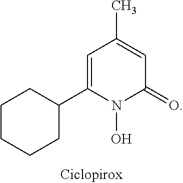

76. The kit of claim 75, wherein ciclopirox is in the form of ciclopirox olamine.

77. The kit of any one of claims 57-64, wherein:
R¹ is hydrogen;
R² is hydrogen;
R³ is a hydroxy group (—OH); and
R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

78. The kit of any one of claims 57-64 and 77, wherein R⁴ is a cycloalkyl or substituted cycloalkyl group having between 5 and 14 carbon atoms.

79. The kit of any one of claims 57-64 and 77-78, wherein R⁴ is a cycloalkyl or substituted cycloalkyl group having 10 carbon atoms.

80. The kit of any one of claims 57-64 and 77-79, wherein R⁴ is

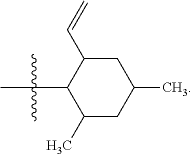 [SMILES: *C1C(C)CC(C)CC1C=C]

81. The kit of any one of claims 57-64 and 77-80, wherein the light-harvesting molecule comprises the structure of pyridoxatin, or an enantiomer, or a tautomer thereof:

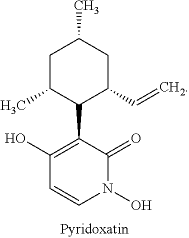

82. The kit of any one of claims 57-81, wherein the rare earth element is at least one selected from the group consisting of Sm, Eu, Tb, Dy, Pr, and Ho.

83. The kit of any one of claims 57-82, wherein the actinide element is Cm and/or Am.

84. The kit of any one of claims 57-83, further comprising a light source that emits ultraviolet (UV) light with a wavelength of less than 400 nm.

85. The kit of any one of claims 57-84, further comprising a luminescence detector.

86. A method for detecting the presence of a rare earth element and/or an actinide element in a test sample, the method comprising:
combining the test sample with the composition of any one of claims 1-26 to form a test mixture;
exposing the test mixture to UV light with a wavelength of less than 400 nm; and
determining that the rare earth element and/or the actinide element is present in the test sample when a luminescent signal is detected by a luminescence detector.

87. The method of claim 86, wherein the luminescent signal is a fluorescence emission.

88. The method of claim 86 or 87, wherein the rare earth element is at least one selected from the group consisting of Sm, Eu, Tb, Dy, Pr, and Ho.

89. The method of any one of claims 86-88, wherein the actinide element is Cm and/or Am.

90. The method of any one of claims 86-88, wherein the rare earth element is Sm, wherein a fluorescence emission at 630-670 nm is detected upon excitation at a wavelength of 290-330 nm.

91. The method of any one of claims 86-88, wherein the rare earth element is Eu, wherein a fluorescence emission at 600-630 nm is detected upon excitation at a wavelength of 290-330 nm.

92. The method of any one of claims 86-88, wherein the rare earth element is Tb, wherein a fluorescence emission at 520-560 nm is detected upon excitation at a wavelength of 290-330 nm.

93. The method of any one of claims 86-88, wherein the rare earth element is Dy, wherein a fluorescence emission at 550-590 nm is detected upon excitation at a wavelength of 290-330 nm.

94. The method of any one of claims 86-93, wherein the test mixture further comprises water or an organic solvent.

95. The method of claim 94, wherein the organic solvent is at least one selected from the group consisting of an alcohol, acetonitrile, dimethylformamide (DMF), and dimethyl sulfoxide (DMSO).

96. The method of any one of claims 86-95, wherein the test mixture has a pH between about 6 to about 11.

97. The method of any one of claims 86-96, wherein the light-harvesting molecule is present in the test mixture at a concentration of 1-50 μM.

98. A method for detecting the presence of a rare earth element and/or an actinide element in a test sample, the method comprising:
combining the test sample with a light-harvesting molecule capable of producing luminescence upon interaction with a rare earth element and/or an actinide element to form a solid test mixture;
exposing the solid test mixture to UV light with a wavelength of less than 400 nm; and
determining that the rare earth element and/or the actinide element is present in the test sample when a luminescent signal is detected by a luminescence detector,
wherein the light-harvesting molecule comprises one hydroxypyridinone (HOPO) group having the structure of formula (I) or (II): 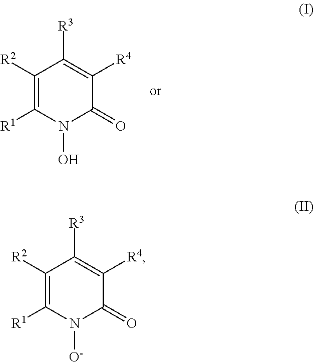
or a salt, solvate, tautomer, or stereoisomer thereof,
wherein each of R¹-R⁴ are independently selected from a hydrogen, a hydroxy group, or a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

99. The method of claim 98, wherein the light-harvesting molecule lacks an amide group.

100. The method of claim 98 or 99, wherein the light-harvesting molecule comprises one hydroxypyridinone (HOPO) group having the structure of formula (I), or a salt, solvate, tautomer, or stereoisomer thereof.

101. The method of claim 98 or 99, wherein the light-harvesting molecule comprises one hydroxypyridinone (HOPO) group having the structure of formula (II), or a salt, solvate, tautomer, or stereoisomer thereof.

102. The method of any one of claims 98-101, wherein:
R¹ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms;
R² is hydrogen;
R³ is an alkyl group having from 1 to 6 carbon atoms; and
R⁴ is hydrogen.

103. The method of any one of claims 98-102, wherein R³ is a methyl group (—CH₃).

104. The method of any one of claims 98-103, wherein R¹ is an alkyl group having between 1 to 10 carbon atoms.

105. The method of any one of claims 98-104, wherein R¹ is an alkyl group having 8 carbon atoms.

106. The method of any one of claims 98-105, wherein R¹ is —CH₂CH(CH₃)CH₂C(CH₃)₃.

107. The method of any one of claims 98-106, wherein the light-harvesting molecule comprises the structure of piroctone or a salt thereof:

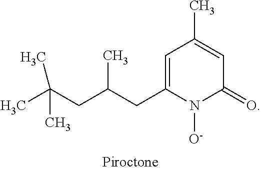 [SMILES: Cc1cc(CC(C)CC(C)(C)C)n([O-])c(=O)c1]

108. The method of claim 107, wherein piroctone is in the form of piroctone olamine.

109. The method of any one of claims 98-103, wherein R¹ is a cycloalkyl or substituted cycloalkyl group having between 3 and 8 carbon atoms.

110. The method of any one of claims 98-103 and 109, wherein R¹ is a cycloalkyl or substituted cycloalkyl group having 6 carbon atoms.

111. The method of any one of claims 98-103 and 109-110, wherein R¹ is cyclohexyl.

112. The method of any one of claims 98-103 and 109-111, wherein the light-harvesting molecule comprises the structure of ciclopirox or a salt thereof:

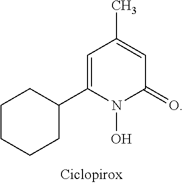

113. The method of claim 112, wherein ciclopirox is in the form of ciclopirox olamine.

114. The method of any one of claims 98-101, wherein:
R¹ is hydrogen;
R² is hydrogen;
R³ is a hydroxy group (—OH); and
R⁴ is a hydrocarbon group, wherein the hydrocarbon group is an alkyl group having from 1 to 20 carbon atoms, an alkylene group having from 2 to 20 carbon atoms, an alkenyl group having from 2 to 20 carbon atoms, an alkynyl group having from 2 to 20 carbon atoms, a substituted alkyl group having from 1 to 20 carbon atoms, a substituted alkylene group having from 2 to 20 carbon atoms, a substituted alkenyl group having from 2 to 20 carbon atoms, a substituted alkynyl group having from 2 to 20 carbon atoms, a cycloalkyl group having from 3 to 20 carbon atoms, a cycloalkenyl group having from 3 to 20 carbon atoms, an aryl group having from 6 to 20 carbon atoms, a substituted cycloalkyl group having from 3 to 20 carbon atoms, a substituted cycloalkenyl group having from 3 to 20 carbon atoms, or a substituted aryl group having from 6 to 20 carbon atoms.

115. The method of any one of claims 98-101 and 114, wherein R⁴ is a cycloalkyl or substituted cycloalkyl group having between 5 and 14 carbon atoms.

116. The method of any one of claims 98-101 and 114-115, wherein R⁴ is a cycloalkyl or substituted cycloalkyl group having 10 carbon atoms.

117. The method of any one of claims 98-101 and 114-116, wherein R⁴ is

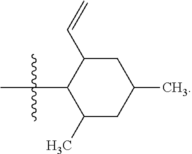 [SMILES: *C1C(C)CC(C)CC1C=C]

118. The method of any one of claims 98-101 and 114-117, wherein the light-harvesting molecule comprises the structure of pyridoxatin, or an enantiomer, or a tautomer thereof:

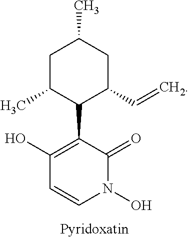

119. The method of any one of claims 98-118, wherein the rare earth element is at least one selected from the group consisting of Sm, Eu, Tb, Dy, Pr, and Ho.

120. The method of any one of claims 98-119, wherein the actinide element is Cm, and/or Am.

121. The method of any one of claims 98-119, wherein the rare earth element is Sm, wherein a fluorescence emission at 630-670 nm is detected upon excitation at a wavelength of 290-330 nm.

122. The method of any one of claims 98-119, wherein the rare earth element is Eu, wherein a fluorescence emission at 600-630 nm is detected upon excitation at a wavelength of 290-330 nm.

123. The method of any one of claims 98-119, wherein the rare earth element is Tb, wherein a fluorescence emission at 520-560 nm is detected upon excitation at a wavelength of 290-330 nm.

124. The method of any one of claims 98-119, wherein the rare earth element is Dy, wherein a fluorescence emission at 550-590 nm is detected upon excitation at a wavelength of 290-330 nm.

125. The method of any one of claims 98-124, wherein the solid test mixture is substantially free of a solvent.
